SEQUENCE LISTING (136 sequences)

SEQ ID NO: 1 - protein, 1228 aa, unidentified
  source 1..1228: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, unidentified
  source 1..1206: note = Lachnospiraceae sp.
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, unidentified
  source 1..1233: note = Lachnospiraceae sp.
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, unidentified
  source 1..1227: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, unidentified
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - DNA, 3987 bp, synthetic construct
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 19 - DNA, 3987 bp, synthetic construct
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 20 - DNA, 3987 bp, synthetic construct
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 21 - DNA, 1592 bp, Medicago truncatula
  source 1..1592: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 22 - DNA, 2000 bp, Zea mays
  source 1..2000: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 23 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - DNA, 621 bp, Petromyzon marinus
  source 1..621: mol_type = genomic DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 26 - protein, 160 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 27 - protein, 207 aa, synthetic construct
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 28 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 29 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 30 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 31 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 32 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 33 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 34 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 35 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 36 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 37 - protein, 1565 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 39 - protein, 167 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 40 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 41 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 42 - protein, 24 aa, synthetic construct
EELLSKNYHLENEVARLKKGSGSG

SEQ ID NO: 43 - protein, 241 aa, synthetic construct
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 44 - protein, 277 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 45 - DNA, 66 bp, Saccharomyces bayanus
  source 1..66: mol_type = genomic DNA
ttcttgtcgtacttatagatcgctacgttatttcaattttgaaaatctgagtcctgggagtgcgga

SEQ ID NO: 46 - protein, 605 aa, Homo sapiens
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 47 - protein, 482 aa, synthetic construct
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 48 - DNA, 10 bp, Methanobacterium thermautotrophicus
  source 1..10: mol_type = genomic DNA
aatttttgga

SEQ ID NO: 49 - protein, 83 aa, Methanobacterium thermautotrophicus
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 50 - DNA, 25 bp, Emesvirus zinderi
  source 1..25: mol_type = genomic DNA
gcgcacatgaggatcacccatgtgc

SEQ ID NO: 51 - protein, 116 aa, Emesvirus zinderi
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 52 - DNA, 26 bp, Pepevirus rubrum
  source 1..26: mol_type = genomic DNA
ataaggagtttatatggaaaccctta

SEQ ID NO: 53 - protein, 127 aa, Pepevirus rubrum
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 54 - DNA, 19 bp, unidentified
  source 1..19: mol_type = genomic DNA; note = Shigella phage
ctgaatgcctgcgagcatc

SEQ ID NO: 55 - protein, 62 aa, unidentified
  source 1..62: note = Shigella phage
MKSIRCKNCNKLLFKADSFDHIEIRCPRCKRHIIMLNACEHPTEKHCGKREKITHSDETVRY

SEQ ID NO: 56 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 57 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 58 - DNA, 4101 bp, synthetic construct
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 59 - DNA, 4101 bp, synthetic construct
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 60 - DNA, 4092 bp, synthetic construct
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 61 - DNA, 4101 bp, synthetic construct
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 62 - DNA, 4101 bp, synthetic construct
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 63 - DNA, 4092 bp, synthetic construct
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 64 - DNA, 4101 bp, synthetic construct
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 65 - DNA, 4101 bp, synthetic construct
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 66 - DNA, 4101 bp, synthetic construct
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 67 - DNA, 3307 bp, synthetic construct
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 68 - DNA, 4101 bp, synthetic construct
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 69 - DNA, 1506 bp, Prunus avium
  source 1..1506: mol_type = genomic DNA
atggcgcctgagaacatgagtatatctgtaaatgggcaatcccaagtccctcctggatttagatttcatccaacagaagaggagctcttgcaatactacttgaagaagaaggtttcaaatcagagtattgatctggatgttatccgagatgttgatctcaataagcttgagccatgggatatacaaggtacataaaaagaccattttctattttctattttctattgtttggtccttgtctagtaatgaagtttggattttggaaccaattaattattcctatatgacccattataagttttcaaattcctttgttttttcctttcagagaagtgtaaaataggaaccacaccgcaaaatgattggtatttcttcagccataaggacaagaagtaccccaccggaacacgaacaaatcgtgcaactgccgccggattctggaaggccactggccgtgataaggtgataagcagtaactgcaggcggatcggtatgaggaagacacttgtgttctacaaaggccgagctccccatggccaaaagtccgattggataatgcatgaatatagacttgatgacaacaacactagcaattgcatcactaatgtaagccactttgtgtacttcaataatcacaccattcttaattatgttctattttgaacaacatagtgattatgttttcaaggaaatattcttttgtcagattgtgtttttgcctgaatgtgcggttcaaatttaaatacaaacatatgcttaagtatgttttatttattttaggtgccaatgattgtgggagaggcagcgcaagaggagggatgggtggtttgccgaatcttcaagaagaaaaacctccataaaattagtccgggcagcacaatcatgagcgccaccacttcatccttgacaacagaaacaagaagtgcccaatccctgtttgattcatgccctgagggagctttggagcaaatgcttcaatacattggaaggacctgcaaagaagaagagaatgaagccaacaacatgagcacaagattcaacctccaacccatcaacaatcacaatggctacaatgagaggttcttgaagcttccaagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctagacaattggcttgcttcagtgacccttacgattgcaatgatgatcatcatgagctccaattaccaacaactctacgatcaatatcttcatcttcatccaacaaatcctaccatgccacccaagactacaacaacagcgagatcaacctctggagctttgctcggcaatcatcatctctgtcgtcctccgacgcgctgtgccacgtgtcgaacggtcctatataa

SEQ ID NO: 70 - DNA, 1191 bp, Prunus avium
  source 1..1191: mol_type = other DNA
atggcgcctgagaacatgagtatatctgtaaatgggcaatcccaagtccctcctggatttagatttcatccaacagaagaggagctcttgcaatactacttgaagaagaaggtttcaaatcagagtattgatctggatgttatccgagatgttgatctcaataagcttgagccatgggatatacaagagaagtgtaaaataggaaccacaccgcaaaatgattggtatttcttcagccataaggacaagaagtaccccaccggaacacgaacaaatcgtgcaactgccgccggattctggaaggccactggccgtgataaggtgataagcagtaactgcaggcggatcggtatgaggaagacacttgtgttctacaaaggccgagctccccatggccaaaagtccgattggataatgcatgaatatagacttgatgacaacaacactagcaattgcatcactaatgtgccaatgattgtgggagaggcagcgcaagaggagggatgggtggtttgccgaatcttcaagaagaaaaacctccataaaattagtccgggcagcacaatcatgagcgccaccacttcatccttgacaacagaaacaagaagtgcccaatccctgtttgattcatgccctgagggagctttggagcaaatgcttcaatacattggaaggacctgcaaagaagaagagaatgaagccaacaacatgagcacaagattcaacctccaacccatcaacaatcacaatggctacaatgagaggttcttgaagcttccaagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctagacaattggcttgcttcagtgacccttacgattgcaatgatgatcatcatgagctccaattaccaacaactctacgatcaatatcttcatcttcatccaacaaatcctaccatgccacccaagactacaacaacagcgagatcaacctctggagctttgctcggcaatcatcatctctgtcgtcctccgacgcgctgtgccacgtgtcgaacggtcctatataa

SEQ ID NO: 71 - protein, 396 aa, Prunus avium
MAPENMSISVNGQSQVPPGFRFHPTEEELLQYYLKKKVSNQSIDLDVIRDVDLNKLEPWDIQEKCKIGTTPQNDWYFFSHKDKKYPTGTRTNRATAAGFWKATGRDKVISSNCRRIGMRKTLVFYKGRAPHGQKSDWIMHEYRLDDNNTSNCITNVPMIVGEAAQEEGWVVCRIFKKKNLHKISPGSTIMSATTSSLTTETRSAQSLFDSCPEGALEQMLQYIGRTCKEEENEANNMSTRFNLQPINNHNGYNERFLKLPSLDSPNSTSSQDCYQPNIHEEMMGAENNIDEDFQHQMDSGLTNWAALDRFVASQLNGQAETSRQLACFSDPYDCNDDHHELQLPTTLRSISSSSSNKSYHATQDYNNSEINLWSFARQSSSLSSSDALCHVSNGPI

SEQ ID NO: 72 - DNA, 187 bp, Prunus avium
  source 1..187: mol_type = genomic DNA
atggcgcctgagaacatgagtatatctgtaaatgggcaatcccaagtccctcctggatttagatttcatccaacagaagaggagctcttgcaatactacttgaagaagaaggtttcaaatcagagtattgatctggatgttatccgagatgttgatctcaataagcttgagccatgggatatacaag

SEQ ID NO: 73 - DNA, 149 bp, Prunus avium
  source 1..149: mol_type = genomic DNA
tggcgcctgagaacatgagtatatctgtaaatgggcaatcccaagtccctcctggatttagatttcatccaacagaagaggagctcttgcaatactacttgaagaagaaggtttcaaatcagagtattgatctggatgttatccgagat

SEQ ID NO: 74 - DNA, 109 bp, Prunus avium
  source 1..109: mol_type = genomic DNA
atatctgtaaatgggcaatcccaagtccctcctggatttagatttcatccaacagaagaggagctcttgcaatactacttgaagaagaaggtttcaaatcagagtattg

SEQ ID NO: 75 - DNA, 69 bp, Prunus avium
  source 1..69: mol_type = genomic DNA
ccaagtccctcctggatttagatttcatccaacagaagaggagctcttgcaatactacttgaagaagaa

SEQ ID NO: 76 - DNA, 29 bp, Prunus avium
  source 1..29: mol_type = genomic DNA
gatttcatccaacagaagaggagctcttg

SEQ ID NO: 77 - protein, 49 aa, Prunus avium
MAPENMSISVNGQSQVPPGFRFHPTEEELLQYYLKKKVSNQSIDLDVIR

SEQ ID NO: 78 - protein, 29 aa, Prunus avium
NGQSQVPPGFRFHPTEEELLQYYLKKKVS

SEQ ID NO: 79 - protein, 19 aa, Prunus avium
VPPGFRFHPTEEELLQYYL

SEQ ID NO: 80 - protein, 5 aa, Prunus avium
EEELL

SEQ ID NO: 81 - DNA, 1504 bp, Prunus persica
  source 1..1504: mol_type = genomic DNA
atggcgcctgagaacatgagtatatctgtaaatgggcaatcccaagtccctcctggctttagatttcatccaacagaagaggagctcttgcaatactacttgaagaagaaggtttcaaatcagagtattgatctggatgttatccgagatgttgatctcaataagcttgagccatgggatatacaaggtacataaaaagaccattttctattttctattgtttggtccttgtctagcaatgaagtttggattttggaaccaattaattattcctatatgacccattataagttctcaaattcctttgttttttcctttcagagaagtgtaaaataggaaccacaccacaaaatgattggtatttcttcagccataaggacaagaagtaccccaccggaacaagaacaaaccgtgcaactgcggccggattctggaaggccactggccgtgataaggtgataagcagtaactgcaggcggatcggtatgaggaagactcttgtgttctacaaaggccgagctccccatggccaaaagtccgattggatcatgcatgaatatagacttgatgacaacaacactagcaattgcatcaccaatgtaagccactttgtgtacttcaataatcacaccattcttaattatgttctattttgaacagcatgcatagtgaaattttctttcctcagattgcgtttttgcctgaatgtgcggttaaaatttacgacaatacaaacatatgcttaagtatgttttgtttattttaggtgccaatgattgtgggagaggcagcacaagaggagggatgggtggtttgccgaatcttcaagaagaaaaacctccataaaattagttcgggcagcacaatcatgagcaccaccacttcatccatgacaacagaaacaagaagtgcccaatccctgtttgattcatgccctgagggagctttggagcaaatgcttcaatacattggaaggacctgcaaggaagaagagaatgaaggcaacaacatgagcacaagattcaacctccaatccatcaacacaggcatcaacaatcacaatggctaccatgagaggttcttgaagcttccgagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctaggcaattggcttgcttcagtgacccttatgattgcaatgatgataatcatgagctccaattaccaacaactctacgatcaatatcttcatcttcatccaacaaatcctaccatgccacccaagactacaacaacggcgagatcaacctctggagctttgctcggcaatcatcatctctgtcgtcctccgacgcgctgtgccacgtgtcgaacggttctatataa

SEQ ID NO: 82 - DNA, 1203 bp, Prunus persica
  source 1..1203: mol_type = other DNA
atggcacctgagaacatgagtatatctgtaaatgggcaatcccaagtccctcctggctttagatttcatccaacagaagaggagctcttgcaatactacttgaagaagaaggtttcaaatcagagtattgatctggatgttatccgagatgttgatctcaataagcttgagccatgggatatacaagagaagtgtaaaataggaaccacaccacaaaatgattggtatttcttcagccataaggacaagaagtaccccaccggaacaagaacaaaccgtgcaactgcggccggattctggaaggccactggccgtgataaggtgataagcagtaactgcaggcggatcggtatgaggaagactcttgtgttctacaaaggccgagctccccatggccaaaagtccgattggatcatgcatgaatatagacttgatgacaacaacactagcaattgcatcaccaatgtgccaatgattgtgggagaggcagcacaagaggagggatgggtggtttgccgaatcttcaagaagaaaaacctccataaaattagttcgggcagcacaatcatgagcaccaccacttcatccatgacaacagaaacaagaagtgcccaatccctgtttgattcatgccctgagggagctttggagcaaatgcttcaatacattggaaggacctgcaaggaagaagagaatgaaggcaacaacatgagcacaagattcaacctccaatccatcaacacaggcatcaacaatcacaatggctaccatgagaggttcttgaagcttccgagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctaggcaattggcttgcttcagtgacccttatgattgcaatgatgataatcatgagctccaattaccaacaactctacgatcaatatcttcatcttcatccaacaaatcctaccatgccacccaagactacaacaacggcgagatcaacctctggagctttgctcggcaatcatcatctctgtcgtcctccgacgcgctgtgccacgtgtcgaacggttctatataa

SEQ ID NO: 83 - protein, 400 aa, Prunus persica
MAPENMSISVNGQSQVPPGFRFHPTEEELLQYYLKKKVSNQSIDLDVIRDVDLNKLEPWDIQEKCKIGTTPQNDWYFFSHKDKKYPTGTRTNRATAAGFWKATGRDKVISSNCRRIGMRKTLVFYKGRAPHGQKSDWIMHEYRLDDNNTSNCITNVPMIVGEAAQEEGWVVCRIFKKKNLHKISSGSTIMSTTTSSMTTETRSAQSLFDSCPEGALEQMLQYIGRTCKEEENEGNNMSTRFNLQSINTGINNHNGYHERFLKLPSLDSPNSTSSQDCYQPNIHEEMMGAENNIDEDFQHQMDSGLTNWAALDRFVASQLNGQAETSRQLACFSDPYDCNDDNHELQLPTTLRSISSSSSNKSYHATQDYNNGEINLWSFARQSSSLSSSDALCHVSNGSI

SEQ ID NO: 84 - DNA, 187 bp, Prunus persica
  source 1..187: mol_type = genomic DNA
atggcgcctgagaacatgagtatatctgtaaatgggcaatcccaagtccctcctggctttagatttcatccaacagaagaggagctcttgcaatactacttgaagaagaaggtttcaaatcagagtattgatctggatgttatccgagatgttgatctcaataagcttgagccatgggatatacaag

SEQ ID NO: 85 - DNA, 149 bp, Prunus persica
  source 1..149: mol_type = genomic DNA
tggcgcctgagaacatgagtatatctgtaaatgggcaatcccaagtccctcctggctttagatttcatccaacagaagaggagctcttgcaatactacttgaagaagaaggtttcaaatcagagtattgatctggatgttatccgagat

SEQ ID NO: 86 - DNA, 110 bp, Prunus persica
  source 1..110: mol_type = genomic DNA
tatatctgtaaatgggcaatcccaagtccctcctggctttagatttcatccaacagaagaggagctcttgcaatactacttgaagaagaaggtttcaaatcagagtattg

SEQ ID NO: 87 - DNA, 69 bp, Prunus persica
  source 1..69: mol_type = genomic DNA
ccaagtccctcctggctttagatttcatccaacagaagaggagctcttgcaatactacttgaagaagaa

SEQ ID NO: 88 - DNA, 1375 bp, Prunus avium
  source 1..1375: mol_type = genomic DNA
atggctgaggatcatatgaatctatcaataaacggccagtctcaagttcctccgggttttcggttccatccaactgaggaggagcttctccattactatctgaggaagaaagtggccttcgagaggattgatcttgatgtgattcgagaagttgatctcaataagctcgagccatgggatattcaaggtacgcattaactcttaatcaactttgattcatatacacgcagtcacaattcatggaggttggtttgttaattaatttttctgacatgatgcagagaagtgcaagataggttccaccccacaaaacgattggtactttttcagtcacaaagacaagaagtacccgaccggaactcgaacgaatcgggcaacggctgccgggttttggaaggccacagggcgggacaagatcatctacagcggctttagaagaattgggttgagaaagacgcttgtgttttacaagggaagagctcctcatgggcaaaagtcggattggatcatgcatgaatataggcttgatgaaagcaacacccatgacaccacagtaagccattgattctccggatatacatacttccatccattcaaacttgatatactcatatattgctatgtgcatatatatatatatatacaggtttgtaattcaatggccgagtcaatggccgaagatgggtgggtggtatgccgagtattcaagaagaagaattatcagaaagccttagagagtcctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagtccaacccttccaacttttgatcaggaccgtagcttcaaagcttgctaccaggccattgatgagatgctcatagacactccttcttcaacaaaccaaccaagcaatggttgtgacaataatgacccagttgaagatcatgatgagtaccccaaaaccaggctaaatgactgggctacccttgaccgccttgtggcatctcagctgggtcaactcaacggccaagatcaagagacatcaaagcacaacctgtcatgctttggtgatccaaacatggctttttgttcttctccttcacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactttgccacttgtcggtataa

SEQ ID NO: 89 - DNA, 1188 bp, Prunus avium
  source 1..1188: mol_type = other DNA
atggctgaggatcatatgaatctatcaataaacggccagtctcaagttcctccgggttttcggttccatccaactgaggaggagcttctccattactatctgaggaagaaagtggccttcgagaggattgatcttgatgtgattcgagaagttgatctcaataagctcgagccatgggatattcaagagaagtgcaagataggttccaccccacaaaacgattggtactttttcagtcacaaagacaagaagtacccgaccggaactcgaacgaatcgggcaacggctgccgggttttggaaggccacagggcgggacaagatcatctacagcggctttagaagaattgggttgagaaagacgcttgtgttttacaagggaagagctcctcatgggcaaaagtcggattggatcatgcatgaatataggcttgatgaaagcaacacccatgacaccacagtttgtaattcaatggccgagtcaatggccgaagatgggtgggtggtatgccgagtattcaagaagaagaattatcagaaagccttagagagtcctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagtccaacccttccaacttttgatcaggaccgtagcttcaaagcttgctaccaggccattgatgagatgctcatagacactccttcttcaacaaaccaaccaagcaatggttgtgacaataatgacccagttgaagatcatgatgagtaccccaaaaccaggctaaatgactgggctacccttgaccgccttgtggcatctcagctgggtcaactcaacggccaagatcaagagacatcaaagcacaacctgtcatgctttggtgatccaaacatggctttttgttcttctccttcacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactttgccacttgtcggtataa

SEQ ID NO: 90 - protein, 395 aa, Prunus avium
MAEDHMNLSINGQSQVPPGFRFHPTEEELLHYYLRKKVAFERIDLDVIREVDLNKLEPWDIQEKCKIGSTPQNDWYFFSHKDKKYPTGTRTNRATAAGFWKATGRDKIIYSGFRRIGLRKTLVFYKGRAPHGQKSDWIMHEYRLDESNTHDTTVCNSMAESMAEDGWVVCRVFKKKNYQKALESPKASFSMESSSNQIHSSRNDGVLDQILMYMGRTCKLENHDQSLNNLSERFMHLPRLESPTLPTFDQDRSFKACYQAIDEMLIDTPSSTNQPSNGCDNNDPVEDHDEYPKTRLNDWATLDRLVASQLGQLNGQDQETSKHNLSCFGDPNMAFCSSPSHDHDHDHDVQLSYPYLRTSRSHHQSEVYSNENDLWNFTKSPSSPSSSDPLCHLSV

SEQ ID NO: 91 - DNA, 187 bp, Prunus avium
  source 1..187: mol_type = genomic DNA
atggctgaggatcatatgaatctatcaataaacggccagtctcaagttcctccgggttttcggttccatccaactgaggaggagcttctccattactatctgaggaagaaagtggccttcgagaggattgatcttgatgtgattcgagaagttgatctcaataagctcgagccatgggatattcaag

SEQ ID NO: 92 - DNA, 166 bp, Prunus avium
  source 1..166: mol_type = genomic DNA
tggctgaggatcatatgaatctatcaataaacggccagtctcaagttcctccgggttttcggttccatccaactgaggaggagcttctccattactatctgaggaagaaagtggccttcgagaggattgatcttgatgtgattcgagaagttgatctcaataagct

SEQ ID NO: 93 - DNA, 126 bp, Prunus avium
  source 1..126: mol_type = genomic DNA
ctatcaataaacggccagtctcaagttcctccgggttttcggttccatccaactgaggaggagcttctccattactatctgaggaagaaagtggccttcgagaggattgatcttgatgtgattcga

SEQ ID NO: 94 - DNA, 86 bp, Prunus avium
  source 1..86: mol_type = genomic DNA
tcaagttcctccgggttttcggttccatccaactgaggaggagcttctccattactatctgaggaagaaagtggccttcgagagga

SEQ ID NO: 95 - DNA, 46 bp, Prunus avium
  source 1..46: mol_type = genomic DNA
ggttccatccaactgaggaggagcttctccattactatctgaggaa

SEQ ID NO: 96 - protein, 49 aa, Prunus avium
MAEDHMNLSINGQSQVPPGFRFHPTEEELLHYYLRKKVAFERIDLDVIR

SEQ ID NO: 97 - protein, 29 aa, Prunus avium
NGQSQVPPGFRFHPTEEELLHYYLRKKVA

SEQ ID NO: 98 - protein, 19 aa, Prunus avium
VPPGFRFHPTEEELLHYYL

SEQ ID NO: 99 - DNA, 1379 bp, Prunus persica
  source 1..1379: mol_type = genomic DNA
atggctgaggatcatatgaatctatcaataaacggccagtctcaagttcctccgggctttcggttccatccaactgaggaggagcttctccattactatctgaggaagaaagtggcctttgagaggattgatcttgatgtgattcgagaagttgatctcaataagctcgagccatgggatattcaaggtacgcattaactcttcatcaactttaattcatatatacgcagtcacaattcatggtggttggtttgttaattaatctttctgacatgatgcagagaagtgcaagataggttccaccccacaaaacgattggtactttttcagtcacaaagacaagaagtacccgaccggaactcgaaccaatcgggcaacggctgccgggttttggaaggccacagggcgggacaagatcatctacagcggctttagaagaattgggttgagaaagacgcttgtgttttacaaggggagagctcctcatgggcaaaagtcggattggatcatgcatgaatataggcttgatgaaagcaacacccatgacaccaccgtaagccattaattctccagatatacatacttccatctattcaaacttgatatactcattcatatattgctatgtgcatatataaatatatatacaggtttgtaattcaatggccgagtcaatggccgaagatgggtgggtggtatgccgagtattcaagaagaagaattatcagaaagccttagagagccctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagcccaactcttccaacttttgatcaggaccgtagcttcaaagcttgctaccaggccattgatgagatgctcatagacactccttcttcaacaaaccaaccaagtaatggttgtgacaataatgacccagttgaagatcatgatgagtaccccaaaaccaggctaaatgactgggctacccttgaccgccttgtggcatctcagctgggtcaactcaacggccaagatcaagagacctcaaagcacaacctgtcatgctttggtgatccaaacatggctttttgttcttctcctccacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactgtgccacttgtctgtataa

SEQ ID NO: 100 - DNA, 1188 bp, Prunus persica
  source 1..1188: mol_type = other DNA
atggctgaggatcatatgaatctatcaataaacggccagtctcaagttcctccgggctttcggttccatccaactgaggaggagcttctccattactatctgaggaagaaagtggcctttgagaggattgatcttgatgtgattcgagaagttgatctcaataagctcgagccatgggatattcaagagaagtgcaagataggttccaccccacaaaacgattggtactttttcagtcacaaagacaagaagtacccgaccggaactcgaaccaatcgggcaacggctgccgggttttggaaggccacagggcgggacaagatcatctacagcggctttagaagaattgggttgagaaagacgcttgtgttttacaaggggagagctcctcatgggcaaaagtcggattggatcatgcatgaatataggcttgatgaaagcaacacccatgacaccaccgtttgtaattcaatggccgagtcaatggccgaagatgggtgggtggtatgccgagtattcaagaagaagaattatcagaaagccttagagagccctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagcccaactcttccaacttttgatcaggaccgtagcttcaaagcttgctaccaggccattgatgagatgctcatagacactccttcttcaacaaaccaaccaagtaatggttgtgacaataatgacccagttgaagatcatgatgagtaccccaaaaccaggctaaatgactgggctacccttgaccgccttgtggcatctcagctgggtcaactcaacggccaagatcaagagacctcaaagcacaacctgtcatgctttggtgatccaaacatggctttttgttcttctcctccacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactgtgccacttgtctgtataa

SEQ ID NO: 101 - protein, 395 aa, Prunus persica
MAEDHMNLSINGQSQVPPGFRFHPTEEELLHYYLRKKVAFERIDLDVIREVDLNKLEPWDIQEKCKIGSTPQNDWYFFSHKDKKYPTGTRTNRATAAGFWKATGRDKIIYSGFRRIGLRKTLVFYKGRAPHGQKSDWIMHEYRLDESNTHDTTVCNSMAESMAEDGWVVCRVFKKKNYQKALESPKASFSMESSSNQIHSSRNDGVLDQILMYMGRTCKLENHDQSLNNLSERFMHLPRLESPTLPTFDQDRSFKACYQAIDEMLIDTPSSTNQPSNGCDNNDPVEDHDEYPKTRLNDWATLDRLVASQLGQLNGQDQETSKHNLSCFGDPNMAFCSSPPHDHDHDHDVQLSYPYLRTSRSHHQSEVYSNENDLWNFTKSPSSPSSSDPLCHLSV

SEQ ID NO: 102 - DNA, 187 bp, Prunus persica
  source 1..187: mol_type = genomic DNA
atggctgaggatcatatgaatctatcaataaacggccagtctcaagttcctccgggctttcggttccatccaactgaggaggagcttctccattactatctgaggaagaaagtggcctttgagaggattgatcttgatgtgattcgagaagttgatctcaataagctcgagccatgggatattcaag

SEQ ID NO: 103 - DNA, 166 bp, Prunus persica
  source 1..166: mol_type = genomic DNA
tggctgaggatcatatgaatctatcaataaacggccagtctcaagttcctccgggctttcggttccatccaactgaggaggagcttctccattactatctgaggaagaaagtggcctttgagaggattgatcttgatgtgattcgagaagttgatctcaataagct

SEQ ID NO: 104 - DNA, 126 bp, Prunus persica
  source 1..126: mol_type = genomic DNA
ctatcaataaacggccagtctcaagttcctccgggctttcggttccatccaactgaggaggagcttctccattactatctgaggaagaaagtggcctttgagaggattgatcttgatgtgattcga

SEQ ID NO: 105 - DNA, 86 bp, Prunus persica
  source 1..86: mol_type = genomic DNA
tcaagttcctccgggctttcggttccatccaactgaggaggagcttctccattactatctgaggaagaaagtggcctttgagagga

SEQ ID NO: 106 - DNA, 1254 bp, Rubus sp.
  source 1..1254: mol_type = other DNA
atgactgaaaacgtgagtatatctgtgaatgggcaatctcaagtccctcctggtttcagatttcatccaactgaagaggagctcttgcagtactacttgaggaagaaggtctccaatcagagcattgatctcgatgtcatctgcgatgtcgatctcaataagcttgagccatgggatatacaagagaagtgtaagataggaaccacaccgcaaaatgattggtatttcttcagccacaaagacaagaaataccctaccggaacacgtacaaaccgtgcaacggctgccggattctggaaggccaccgggcgtgataagatgataagcagcaactgcaggcggatcggtatgaggaagactcttgtgttctacaaaggccgagctccccacggccaaaagtccgattggatcatgcacgagtatagactcgacgacaacactagcaacatcactaatgtctcgatcaccgttgtgggagaggcagcggaggaggggtgggtggtttgcagaatcttcaagaagaaaaatctccacaagagcttgagtagtccaatcatgagcacttctatttcttcatctatcacagaagaaattacaagaagctcaagccaaatgtttgatgatgatcatgagggagcttttgaggaaatgcttcagtacataggatcaaggacctgcaaggaagagaataatgaacattatcatcttcatcccatcaacacaggtaatagtgataagattaatggctaccatgatcatggtgataggttcttgaaacttccgagccttgagagccccaactccaccagtagccaaaacggttatcagccaattatcagtcatgaagacatggtcatagatcagaatgagggcacaattgctaaccagcatctgggttatcagcacgtggacgactcagccagcctcaccaactgggcagcccttgatcgtttggtggcttcacaactcaatggagtccatgaaacggggtcgtctaggcaattttcttgcttaattgagcccattcgtgatgtttactgcactagtattactgataatgagcttcagttaccgagtacattacgatcgtcaacttcttcgaattcatccagcaaatcataccacgccactcaaccggaatgcaacaatagcgagattgacctgtggactacttttgctgctgctcgactacaatcatcattccctttgtcatcctccgacgcactatgccacgtgtcgaacgctcctatataa

SEQ ID NO: 107 - protein, 417 aa, Rubus sp.
MTENVSISVNGQSQVPPGFRFHPTEEELLQYYLRKKVSNQSIDLDVICDVDLNKLEPWDIQEKCKIGTTPQNDWYFFSHKDKKYPTGTRTNRATAAGFWKATGRDKMISSNCRRIGMRKTLVFYKGRAPHGQKSDWIMHEYRLDDNTSNITNVSITVVGEAAEEGWVVCRIFKKKNLHKSLSSPIMSTSISSSITEEITRSSSQMFDDDHEGAFEEMLQYIGSRTCKEENNEHYHLHPINTGNSDKINGYHDHGDRFLKLPSLESPNSTSSQNGYQPIISHEDMVIDQNEGTIANQHLGYQHVDDSASLTNWAALDRLVASQLNGVHETGSSRQFSCLIEPIRDVYCTSITDNELQLPSTLRSSTSSNSSSKSYHATQPECNNSEIDLWTTFAAARLQSSFPLSSSDALCHVSNAPI

SEQ ID NO: 108 - DNA, 1191 bp, Rubus sp.
  source 1..1191: mol_type = other DNA
atgtctgcagaggatcatatgaatctatccataaatggtcagtctcaggtccctcctggctttcgcttccatccaaccgaagaggagcttcttcactactatctcaggaagaaagtttcatatgagaggattgatcttgacgtcattcgagaggttgatcttaacaagctcgagccatgggacattcaagagaagtgcaagataggttccaccccacagaacgattggtatttcttcagccacaaagacaagaaataccctacaggtactcgaaccaatcgcgcaaccgctgcagggttttggaaggccaccggccgcgacaagatcatctacagcggcattcgaagaatcggattgagaaagacgcttgtgttttacaagggacgagctccgcacggacaaaagtcagattggatcatgcatgaatacagactagatgaaagcagcagcagcagcagccttgacaccacagtttctagttcaattggggagtcaatgtcagaagatgggtgggtggtttgcagggtcttcaagaagaagaactaccagaaagccttggagagcccaaaatcctcctacccgatggactcatcaaacaaccaaatgctaccgggtggttcgagaaacgacggcgttgtcgatcagattctcttgtacatgggaaggactagttgcaagctcgaaaaccatgactcactccatgacagattcatgcatcttccaaggctcgagagccaaacttctcttccttcacttgcggtccacttcgatcatgaccgtagcttcaaaggttgttatcatgatcagaccattgatgacatgctcatagaaacagatcatcaacaaccttctccaacaacaaaccaacccaatgacccggttgatcatgacgaccccaaaacaaggattgtaaatgactgggctactatccatcagctcgtggcctctcaactcaatggccatgaagacacttcaaagcacttgtcctgcttcggtgacccaaacatggccttttgttcttctcctccttctaatgatgaggacttacagttgtcctatccatatctacgtccaggtagaatatcacagaatcaacccgaagtctacaacagcgagaacgatctatggagcttcacaaaatcgtcgccgtcgccgtcatcatcagacccattatgccacctgtcggtgtaa

SEQ ID NO: 109 - protein, 396 aa, Rubus sp.
MSAEDHMNLSINGQSQVPPGFRFHPTEEELLHYYLRKKVSYERIDLDVIREVDLNKLEPWDIQEKCKIGSTPQNDWYFFSHKDKKYPTGTRTNRATAAGFWKATGRDKIIYSGIRRIGLRKTLVFYKGRAPHGQKSDWIMHEYRLDESSSSSSLDTTVSSSIGESMSEDGWVVCRVFKKKNYQKALESPKSSYPMDSSNNQMLPGGSRNDGVVDQILLYMGRTSCKLENHDSLHDRFMHLPRLESQTSLPSLAVHFDHDRSFKGCYHDQTIDDMLIETDHQQPSPTTNQPNDPVDHDDPKTRIVNDWATIHQLVASQLNGHEDTSKHLSCFGDPNMAFCSSPPSNDEDLQLSYPYLRPGRISQNQPEVYNSENDLWSFTKSSPSPSSSDPLCHLSV

SEQ ID NO: 110 - DNA, 23 bp, synthetic construct
  source 1..23: mol_type = other DNA
gatttcatccaacagaagaggag

SEQ ID NO: 111 - DNA, 23 bp, synthetic construct
  source 1..23: mol_type = other DNA
atccaacagaagaggagctcttg

SEQ ID NO: 112 - DNA, 23 bp, synthetic construct
  source 1..23: mol_type = other DNA
ggttccatccaactgaggaggag

SEQ ID NO: 113 - DNA, 23 bp, synthetic construct
  source 1..23: mol_type = other DNA
ttcctcagatagtaatggagaag

SEQ ID NO: 114 - protein, 29 aa, synthetic construct
MAPENMSISVNGQSQVPPGFRFHPTEEEL

SEQ ID NO: 115 - protein, 393 aa, synthetic construct
MAPENMSISVNGQSQVPPGFRFHPTEEQYYLKKKVSNQSIDLDVIRDVDLNKLEPWDIQEKCKIGTTPQNDWYFFSHKDKKYPTGTRTNRATAAGFWKATGRDKVISSNCRRIGMRKTLVFYKGRAPHGQKSDWIMHEYRLDDNNTSNCITNVPMIVGEAAQEEGWVVCRIFKKKNLHKISPGSTIMSATTSSLTTETRSAQSLFDSCPEGALEQMLQYIGRTCKEEENEANNMSTRFNLQPINNHNGYNERFLKLPSLDSPNSTSSQDCYQPNIHEEMMGAENNIDEDFQHQMDSGLTNWAALDRFVASQLNGQAETSRQLACFSDPYDCNDDHHELQLPTTLRSISSSSSNKSYHATQDYNNSEINLWSFARQSSSLSSSDALCHVSNGPI

SEQ ID NO: 116 - protein, 25 aa, synthetic construct
MAEDHMNLSINGQSQVPPGFRFHPT

SEQ ID NO: 117 - protein, 392 aa, synthetic construct
MAEDHMNLSINGQSQVPPGFRFHPTLLHYYLRKKVAFERIDLDVIREVDLNKLEPWDIQEKCKIGSTPQNDWYFFSHKDKKYPTGTRTNRATAAGFWKATGRDKIIYSGFRRIGLRKTLVFYKGRAPHGQKSDWIMHEYRLDESNTHDTTVCNSMAESMAEDGWVVCRVFKKKNYQKALESPKASFSMESSSNQIHSSRNDGVLDQILMYMGRTCKLENHDQSLNNLSERFMHLPRLESPTLPTFDQDRSFKACYQAIDEMLIDTPSSTNQPSNGCDNNDPVEDHDEYPKTRLNDWATLDRLVASQLGQLNGQDQETSKHNLSCFGDPNMAFCSSPSHDHDHDHDVQLSYPYLRTSRSHHQSEVYSNENDLWNFTKSPSSPSSSDPLCHLSV

SEQ ID NO: 118 - protein, 25 aa, synthetic construct
MAPENMSISVNGQSQVPPGFRFHPT

SEQ ID NO: 119 - protein, 397 aa, synthetic construct
MAPENMSISVNGQSQVPPGFRFHPTEEQYYLKKKVSNQSIDLDVIRDVDLNKLEPWDIQEKCKIGTTPQNDWYFFSHKDKKYPTGTRTNRATAAGFWKATGRDKVISSNCRRIGMRKTLVFYKGRAPHGQKSDWIMHEYRLDDNNTSNCITNVPMIVGEAAQEEGWVVCRIFKKKNLHKISSGSTIMSTTTSSMTTETRSAQSLFDSCPEGALEQMLQYIGRTCKEEENEGNNMSTRFNLQSINTGINNHNGYHERFLKLPSLDSPNSTSSQDCYQPNIHEEMMGAENNIDEDFQHQMDSGLTNWAALDRFVASQLNGQAETSRQLACFSDPYDCNDDNHELQLPTTLRSISSSSSNKSYHATQDYNNGEINLWSFARQSSSLSSSDALCHVSNGSI

SEQ ID NO: 120 - protein, 392 aa, synthetic construct
MAEDHMNLSINGQSQVPPGFRFHPTEEHYYLRKKVAFERIDLDVIREVDLNKLEPWDIQEKCKIGSTPQNDWYFFSHKDKKYPTGTRTNRATAAGFWKATGRDKIIYSGFRRIGLRKTLVFYKGRAPHGQKSDWIMHEYRLDESNTHDTTVCNSMAESMAEDGWVVCRVFKKKNYQKALESPKASFSMESSSNQIHSSRNDGVLDQILMYMGRTCKLENHDQSLNNLSERFMHLPRLESPTLPTFDQDRSFKACYQAIDEMLIDTPSSTNQPSNGCDNNDPVEDHDEYPKTRLNDWATLDRLVASQLGQLNGQDQETSKHNLSCFGDPNMAFCSSPPHDHDHDHDVQLSYPYLRTSRSHHQSEVYSNENDLWNFTKSPSSPSSSDPLCHLSV

SEQ ID NO: 121 - protein, 24 aa, synthetic construct
MTENVSISVNGQSQVPPGFRFHPT

SEQ ID NO: 122 - protein, 414 aa, synthetic construct
MTENVSISVNGQSQVPPGFRFHPTEEQYYLRKKVSNQSIDLDVICDVDLNKLEPWDIQEKCKIGTTPQNDWYFFSHKDKKYPTGTRTNRATAAGFWKATGRDKMISSNCRRIGMRKTLVFYKGRAPHGQKSDWIMHEYRLDDNTSNITNVSITVVGEAAEEGWVVCRIFKKKNLHKSLSSPIMSTSISSSITEEITRSSSQMFDDDHEGAFEEMLQYIGSRTCKEENNEHYHLHPINTGNSDKINGYHDHGDRFLKLPSLESPNSTSSQNGYQPIISHEDMVIDQNEGTIANQHLGYQHVDDSASLTNWAALDRLVASQLNGVHETGSSRQFSCLIEPIRDVYCTSITDNELQLPSTLRSSTSSNSSSKSYHATQPECNNSEIDLWTTFAAARLQSSFPLSSSDALCHVSNAPI

SEQ ID NO: 123 - protein, 26 aa, synthetic construct
MSAEDHMNLSINGQSQVPPGFRFHPT

SEQ ID NO: 124 - protein, 393 aa, synthetic construct
MSAEDHMNLSINGQSQVPPGFRFHPTLLHYYLRKKVSYERIDLDVIREVDLNKLEPWDIQEKCKIGSTPQNDWYFFSHKDKKYPTGTRTNRATAAGFWKATGRDKIIYSGIRRIGLRKTLVFYKGRAPHGQKSDWIMHEYRLDESSSSSSLDTTVSSSIGESMSEDGWVVCRVFKKKNYQKALESPKSSYPMDSSNNQMLPGGSRNDGVVDQILLYMGRTSCKLENHDSLHDRFMHLPRLESQTSLPSLAVHFDHDRSFKGCYHDQTIDDMLIETDHQQPSPTTNQPNDPVDHDDPKTRIVNDWATIHQLVASQLNGHEDTSKHLSCFGDPNMAFCSSPPSNDEDLQLSYPYLRPGRISQNQPEVYNSENDLWSFTKSSPSPSSSDPLCHLSV

SEQ ID NO: 125 - DNA, 1191 bp, synthetic construct
  source 1..1191: mol_type = other DNA
atggcgcctgagaacatgagtatatctgtaaatgggcaatcccaagtccctcctggatttagatttcatccaacagaagaggagctctgatagtactacttgaagaagaaggtttcaaatcagagtattgatctggatgttatccgagatgttgatctcaataagcttgagccatgggatatacaagagaagtgtaaaataggaaccacaccgcaaaatgattggtatttcttcagccataaggacaagaagtaccccaccggaacacgaacaaatcgtgcaactgccgccggattctggaaggccactggccgtgataaggtgataagcagtaactgcaggcggatcggtatgaggaagacacttgtgttctacaaaggccgagctccccatggccaaaagtccgattggataatgcatgaatatagacttgatgacaacaacactagcaattgcatcactaatgtgccaatgattgtgggagaggcagcgcaagaggagggatgggtggtttgccgaatcttcaagaagaaaaacctccataaaattagtccgggcagcacaatcatgagcgccaccacttcatccttgacaacagaaacaagaagtgcccaatccctgtttgattcatgccctgagggagctttggagcaaatgcttcaatacattggaaggacctgcaaagaagaagagaatgaagccaacaacatgagcacaagattcaacctccaacccatcaacaatcacaatggctacaatgagaggttcttgaagcttccaagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctagacaattggcttgcttcagtgacccttacgattgcaatgatgatcatcatgagctccaattaccaacaactctacgatcaatatcttcatcttcatccaacaaatcctaccatgccacccaagactacaacaacagcgagatcaacctctggagctttgctcggcaatcatcatctctgtcgtcctccgacgcgctgtgccacgtgtcgaacggtcctatataa

SEQ ID NO: 126 - DNA, 1182 bp, synthetic construct
  source 1..1182: mol_type = other DNA
atggcgcctgagaacatgagtatatctgtaaatgggcaatcccaagtccctcctggatttagatttcatccaacagaagagcaatactacttgaagaagaaggtttcaaatcagagtattgatctggatgttatccgagatgttgatctcaataagcttgagccatgggatatacaagagaagtgtaaaataggaaccacaccgcaaaatgattggtatttcttcagccataaggacaagaagtaccccaccggaacacgaacaaatcgtgcaactgccgccggattctggaaggccactggccgtgataaggtgataagcagtaactgcaggcggatcggtatgaggaagacacttgtgttctacaaaggccgagctccccatggccaaaagtccgattggataatgcatgaatatagacttgatgacaacaacactagcaattgcatcactaatgtgccaatgattgtgggagaggcagcgcaagaggagggatgggtggtttgccgaatcttcaagaagaaaaacctccataaaattagtccgggcagcacaatcatgagcgccaccacttcatccttgacaacagaaacaagaagtgcccaatccctgtttgattcatgccctgagggagctttggagcaaatgcttcaatacattggaaggacctgcaaagaagaagagaatgaagccaacaacatgagcacaagattcaacctccaacccatcaacaatcacaatggctacaatgagaggttcttgaagcttccaagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctagacaattggcttgcttcagtgacccttacgattgcaatgatgatcatcatgagctccaattaccaacaactctacgatcaatatcttcatcttcatccaacaaatcctaccatgccacccaagactacaacaacagcgagatcaacctctggagctttgctcggcaatcatcatctctgtcgtcctccgacgcgctgtgccacgtgtcgaacggtcctatataa

SEQ ID NO: 127 - DNA, 1188 bp, synthetic construct
  source 1..1188: mol_type = other DNA
atggctgaggatcatatgaatctatcaataaacggccagtctcaagttcctccgggttttcggttccatccaacttgataggagcttctccattactatctgaggaagaaagtggccttcgagaggattgatcttgatgtgattcgagaagttgatctcaataagctcgagccatgggatattcaagagaagtgcaagataggttccaccccacaaaacgattggtactttttcagtcacaaagacaagaagtacccgaccggaactcgaacgaatcgggcaacggctgccgggttttggaaggccacagggcgggacaagatcatctacagcggctttagaagaattgggttgagaaagacgcttgtgttttacaagggaagagctcctcatgggcaaaagtcggattggatcatgcatgaatataggcttgatgaaagcaacacccatgacaccacagtttgtaattcaatggccgagtcaatggccgaagatgggtgggtggtatgccgagtattcaagaagaagaattatcagaaagccttagagagtcctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagtccaacccttccaacttttgatcaggaccgtagcttcaaagcttgctaccaggccattgatgagatgctcatagacactccttcttcaacaaaccaaccaagcaatggttgtgacaataatgacccagttgaagatcatgatgagtaccccaaaaccaggctaaatgactgggctacccttgaccgccttgtggcatctcagctgggtcaactcaacggccaagatcaagagacatcaaagcacaacctgtcatgctttggtgatccaaacatggctttttgttcttctccttcacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactttgccacttgtcggtataa

SEQ ID NO: 128 - DNA, 1179 bp, synthetic construct
  source 1..1179: mol_type = other DNA
atggctgaggatcatatgaatctatcaataaacggccagtctcaagttcctccgggttttcggttccatccaactcttctccattactatctgaggaagaaagtggccttcgagaggattgatcttgatgtgattcgagaagttgatctcaataagctcgagccatgggatattcaagagaagtgcaagataggttccaccccacaaaacgattggtactttttcagtcacaaagacaagaagtacccgaccggaactcgaacgaatcgggcaacggctgccgggttttggaaggccacagggcgggacaagatcatctacagcggctttagaagaattgggttgagaaagacgcttgtgttttacaagggaagagctcctcatgggcaaaagtcggattggatcatgcatgaatataggcttgatgaaagcaacacccatgacaccacagtttgtaattcaatggccgagtcaatggccgaagatgggtgggtggtatgccgagtattcaagaagaagaattatcagaaagccttagagagtcctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagtccaacccttccaacttttgatcaggaccgtagcttcaaagcttgctaccaggccattgatgagatgctcatagacactccttcttcaacaaaccaaccaagcaatggttgtgacaataatgacccagttgaagatcatgatgagtaccccaaaaccaggctaaatgactgggctacccttgaccgccttgtggcatctcagctgggtcaactcaacggccaagatcaagagacatcaaagcacaacctgtcatgctttggtgatccaaacatggctttttgttcttctccttcacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactttgccacttgtcggtataa

SEQ ID NO: 129 - DNA, 1203 bp, synthetic construct
  source 1..1203: mol_type = other DNA
atggcacctgagaacatgagtatatctgtaaatgggcaatcccaagtccctcctggctttagatttcatccaacatgataggagctcttgcaatactacttgaagaagaaggtttcaaatcagagtattgatctggatgttatccgagatgttgatctcaataagcttgagccatgggatatacaagagaagtgtaaaataggaaccacaccacaaaatgattggtatttcttcagccataaggacaagaagtaccccaccggaacaagaacaaaccgtgcaactgcggccggattctggaaggccactggccgtgataaggtgataagcagtaactgcaggcggatcggtatgaggaagactcttgtgttctacaaaggccgagctccccatggccaaaagtccgattggatcatgcatgaatatagacttgatgacaacaacactagcaattgcatcaccaatgtgccaatgattgtgggagaggcagcacaagaggagggatgggtggtttgccgaatcttcaagaagaaaaacctccataaaattagttcgggcagcacaatcatgagcaccaccacttcatccatgacaacagaaacaagaagtgcccaatccctgtttgattcatgccctgagggagctttggagcaaatgcttcaatacattggaaggacctgcaaggaagaagagaatgaaggcaacaacatgagcacaagattcaacctccaatccatcaacacaggcatcaacaatcacaatggctaccatgagaggttcttgaagcttccgagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctaggcaattggcttgcttcagtgacccttatgattgcaatgatgataatcatgagctccaattaccaacaactctacgatcaatatcttcatcttcatccaacaaatcctaccatgccacccaagactacaacaacggcgagatcaacctctggagctttgctcggcaatcatcatctctgtcgtcctccgacgcgctgtgccacgtgtcgaacggttctatataa

SEQ ID NO: 130 - DNA, 1194 bp, synthetic construct
  source 1..1194: mol_type = other DNA
atggcacctgagaacatgagtatatctgtaaatgggcaatcccaagtccctcctggctttagatttcatccaacagaagagcaatactacttgaagaagaaggtttcaaatcagagtattgatctggatgttatccgagatgttgatctcaataagcttgagccatgggatatacaagagaagtgtaaaataggaaccacaccacaaaatgattggtatttcttcagccataaggacaagaagtaccccaccggaacaagaacaaaccgtgcaactgcggccggattctggaaggccactggccgtgataaggtgataagcagtaactgcaggcggatcggtatgaggaagactcttgtgttctacaaaggccgagctccccatggccaaaagtccgattggatcatgcatgaatatagacttgatgacaacaacactagcaattgcatcaccaatgtgccaatgattgtgggagaggcagcacaagaggagggatgggtggtttgccgaatcttcaagaagaaaaacctccataaaattagttcgggcagcacaatcatgagcaccaccacttcatccatgacaacagaaacaagaagtgcccaatccctgtttgattcatgccctgagggagctttggagcaaatgcttcaatacattggaaggacctgcaaggaagaagagaatgaaggcaacaacatgagcacaagattcaacctccaatccatcaacacaggcatcaacaatcacaatggctaccatgagaggttcttgaagcttccgagcctagacagcccaaactccaccagcagccaagactgctaccaacccaatattcatgaggagatgatgggcgcagagaataatattgatgaggattttcagcaccaaatggactcagggctcaccaactgggcagccctcgatcgctttgtggcctcacaactgaatggccaagccgagacctctaggcaattggcttgcttcagtgacccttatgattgcaatgatgataatcatgagctccaattaccaacaactctacgatcaatatcttcatcttcatccaacaaatcctaccatgccacccaagactacaacaacggcgagatcaacctctggagctttgctcggcaatcatcatctctgtcgtcctccgacgcgctgtgccacgtgtcgaacggttctatataa

SEQ ID NO: 131 - DNA, 1188 bp, synthetic construct
  source 1..1188: mol_type = other DNA
atggctgaggatcatatgaatctatcaataaacggccagtctcaagttcctccgggctttcggttccatccaacttgataggagcttctccattactatctgaggaagaaagtggcctttgagaggattgatcttgatgtgattcgagaagttgatctcaataagctcgagccatgggatattcaagagaagtgcaagataggttccaccccacaaaacgattggtactttttcagtcacaaagacaagaagtacccgaccggaactcgaaccaatcgggcaacggctgccgggttttggaaggccacagggcgggacaagatcatctacagcggctttagaagaattgggttgagaaagacgcttgtgttttacaaggggagagctcctcatgggcaaaagtcggattggatcatgcatgaatataggcttgatgaaagcaacacccatgacaccaccgtttgtaattcaatggccgagtcaatggccgaagatgggtgggtggtatgccgagtattcaagaagaagaattatcagaaagccttagagagccctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagcccaactcttccaacttttgatcaggaccgtagcttcaaagcttgctaccaggccattgatgagatgctcatagacactccttcttcaacaaaccaaccaagtaatggttgtgacaataatgacccagttgaagatcatgatgagtaccccaaaaccaggctaaatgactgggctacccttgaccgccttgtggcatctcagctgggtcaactcaacggccaagatcaagagacctcaaagcacaacctgtcatgctttggtgatccaaacatggctttttgttcttctcctccacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactgtgccacttgtctgtataa

SEQ ID NO: 132 - DNA, 1179 bp, synthetic construct
  source 1..1179: mol_type = other DNA
atggctgaggatcatatgaatctatcaataaacggccagtctcaagttcctccgggctttcggttccatccaactgaggagcattactatctgaggaagaaagtggcctttgagaggattgatcttgatgtgattcgagaagttgatctcaataagctcgagccatgggatattcaagagaagtgcaagataggttccaccccacaaaacgattggtactttttcagtcacaaagacaagaagtacccgaccggaactcgaaccaatcgggcaacggctgccgggttttggaaggccacagggcgggacaagatcatctacagcggctttagaagaattgggttgagaaagacgcttgtgttttacaaggggagagctcctcatgggcaaaagtcggattggatcatgcatgaatataggcttgatgaaagcaacacccatgacaccaccgtttgtaattcaatggccgagtcaatggccgaagatgggtgggtggtatgccgagtattcaagaagaagaattatcagaaagccttagagagccctaaagcctccttctccatggaatcatcaagcaaccaaatacacagttccagaaacgatggggtgcttgatcagatactcatgtacatgggaaggacttgcaagctcgaaaaccatgaccaatccttgaataacttatcagaaagatttatgcatctcccaaggctcgagagcccaactcttccaacttttgatcaggaccgtagcttcaaagcttgctaccaggccattgatgagatgctcatagacactccttcttcaacaaaccaaccaagtaatggttgtgacaataatgacccagttgaagatcatgatgagtaccccaaaaccaggctaaatgactgggctacccttgaccgccttgtggcatctcagctgggtcaactcaacggccaagatcaagagacctcaaagcacaacctgtcatgctttggtgatccaaacatggctttttgttcttctcctccacatgatcatgatcatgaccatgacgtacaactatcatatccatacctacgtacaagtagatcccatcaccaatctgaagtctacagcaacgagaatgatctttggaacttcaccaaatcgccgtcatcgccgtcgtcgtcagacccactgtgccacttgtctgtataa

SEQ ID NO: 133 - DNA, 1254 bp, synthetic construct
  source 1..1254: mol_type = other DNA
atgactgaaaacgtgagtatatctgtgaatgggcaatctcaagtccctcctggtttcagatttcatccaacttgataggagctcttgcagtactacttgaggaagaaggtctccaatcagagcattgatctcgatgtcatctgcgatgtcgatctcaataagcttgagccatgggatatacaagagaagtgtaagataggaaccacaccgcaaaatgattggtatttcttcagccacaaagacaagaaataccctaccggaacacgtacaaaccgtgcaacggctgccggattctggaaggccaccgggcgtgataagatgataagcagcaactgcaggcggatcggtatgaggaagactcttgtgttctacaaaggccgagctccccacggccaaaagtccgattggatcatgcacgagtatagactcgacgacaacactagcaacatcactaatgtctcgatcaccgttgtgggagaggcagcggaggaggggtgggtggtttgcagaatcttcaagaagaaaaatctccacaagagcttgagtagtccaatcatgagcacttctatttcttcatctatcacagaagaaattacaagaagctcaagccaaatgtttgatgatgatcatgagggagcttttgaggaaatgcttcagtacataggatcaaggacctgcaaggaagagaataatgaacattatcatcttcatcccatcaacacaggtaatagtgataagattaatggctaccatgatcatggtgataggttcttgaaacttccgagccttgagagccccaactccaccagtagccaaaacggttatcagccaattatcagtcatgaagacatggtcatagatcagaatgagggcacaattgctaaccagcatctgggttatcagcacgtggacgactcagccagcctcaccaactgggcagcccttgatcgtttggtggcttcacaactcaatggagtccatgaaacggggtcgtctaggcaattttcttgcttaattgagcccattcgtgatgtttactgcactagtattactgataatgagcttcagttaccgagtacattacgatcgtcaacttcttcgaattcatccagcaaatcataccacgccactcaaccggaatgcaacaatagcgagattgacctgtggactacttttgctgctgctcgactacaatcatcattccctttgtcatcctccgacgcactatgccacgtgtcgaacgctcctatataa

SEQ ID NO: 134 - DNA, 1245 bp, synthetic construct
  source 1..1245: mol_type = other DNA
atgactgaaaacgtgagtatatctgtgaatgggcaatctcaagtccctcctggtttcagatttcatccaactgaagagcagtactacttgaggaagaaggtctccaatcagagcattgatctcgatgtcatctgcgatgtcgatctcaataagcttgagccatgggatatacaagagaagtgtaagataggaaccacaccgcaaaatgattggtatttcttcagccacaaagacaagaaataccctaccggaacacgtacaaaccgtgcaacggctgccggattctggaaggccaccgggcgtgataagatgataagcagcaactgcaggcggatcggtatgaggaagactcttgtgttctacaaaggccgagctccccacggccaaaagtccgattggatcatgcacgagtatagactcgacgacaacactagcaacatcactaatgtctcgatcaccgttgtgggagaggcagcggaggaggggtgggtggtttgcagaatcttcaagaagaaaaatctccacaagagcttgagtagtccaatcatgagcacttctatttcttcatctatcacagaagaaattacaagaagctcaagccaaatgtttgatgatgatcatgagggagcttttgaggaaatgcttcagtacataggatcaaggacctgcaaggaagagaataatgaacattatcatcttcatcccatcaacacaggtaatagtgataagattaatggctaccatgatcatggtgataggttcttgaaacttccgagccttgagagccccaactccaccagtagccaaaacggttatcagccaattatcagtcatgaagacatggtcatagatcagaatgagggcacaattgctaaccagcatctgggttatcagcacgtggacgactcagccagcctcaccaactgggcagcccttgatcgtttggtggcttcacaactcaatggagtccatgaaacggggtcgtctaggcaattttcttgcttaattgagcccattcgtgatgtttactgcactagtattactgataatgagcttcagttaccgagtacattacgatcgtcaacttcttcgaattcatccagcaaatcataccacgccactcaaccggaatgcaacaatagcgagattgacctgtggactacttttgctgctgctcgactacaatcatcattccctttgtcatcctccgacgcactatgccacgtgtcgaacgctcctatataa

SEQ ID NO: 135 - DNA, 1191 bp, synthetic construct
  source 1..1191: mol_type = other DNA
atgtctgcagaggatcatatgaatctatccataaatggtcagtctcaggtccctcctggctttcgcttccatccaacctgataggagcttcttcactactatctcaggaagaaagtttcatatgagaggattgatcttgacgtcattcgagaggttgatcttaacaagctcgagccatgggacattcaagagaagtgcaagataggttccaccccacagaacgattggtatttcttcagccacaaagacaagaaataccctacaggtactcgaaccaatcgcgcaaccgctgcagggttttggaaggccaccggccgcgacaagatcatctacagcggcattcgaagaatcggattgagaaagacgcttgtgttttacaagggacgagctccgcacggacaaaagtcagattggatcatgcatgaatacagactagatgaaagcagcagcagcagcagccttgacaccacagtttctagttcaattggggagtcaatgtcagaagatgggtgggtggtttgcagggtcttcaagaagaagaactaccagaaagccttggagagcccaaaatcctcctacccgatggactcatcaaacaaccaaatgctaccgggtggttcgagaaacgacggcgttgtcgatcagattctcttgtacatgggaaggactagttgcaagctcgaaaaccatgactcactccatgacagattcatgcatcttccaaggctcgagagccaaacttctcttccttcacttgcggtccacttcgatcatgaccgtagcttcaaaggttgttatcatgatcagaccattgatgacatgctcatagaaacagatcatcaacaaccttctccaacaacaaaccaacccaatgacccggttgatcatgacgaccccaaaacaaggattgtaaatgactgggctactatccatcagctcgtggcctctcaactcaatggccatgaagacacttcaaagcacttgtcctgcttcggtgacccaaacatggccttttgttcttctcctccttctaatgatgaggacttacagttgtcctatccatatctacgtccaggtagaatatcacagaatcaacccgaagtctacaacagcgagaacgatctatggagcttcacaaaatcgtcgccgtcgccgtcatcatcagacccattatgccacctgtcggtgtaa

SEQ ID NO: 136 - DNA, 1182 bp, synthetic construct
  source 1..1182: mol_type = other DNA
atgtctgcagaggatcatatgaatctatccataaatggtcagtctcaggtccctcctggctttcgcttccatccaacccttcttcactactatctcaggaagaaagtttcatatgagaggattgatcttgacgtcattcgagaggttgatcttaacaagctcgagccatgggacattcaagagaagtgcaagataggttccaccccacagaacgattggtatttcttcagccacaaagacaagaaataccctacaggtactcgaaccaatcgcgcaaccgctgcagggttttggaaggccaccggccgcgacaagatcatctacagcggcattcgaagaatcggattgagaaagacgcttgtgttttacaagggacgagctccgcacggacaaaagtcagattggatcatgcatgaatacagactagatgaaagcagcagcagcagcagccttgacaccacagtttctagttcaattggggagtcaatgtcagaagatgggtgggtggtttgcagggtcttcaagaagaagaactaccagaaagccttggagagcccaaaatcctcctacccgatggactcatcaaacaaccaaatgctaccgggtggttcgagaaacgacggcgttgtcgatcagattctcttgtacatgggaaggactagttgcaagctcgaaaaccatgactcactccatgacagattcatgcatcttccaaggctcgagagccaaacttctcttccttcacttgcggtccacttcgatcatgaccgtagcttcaaaggttgttatcatgatcagaccattgatgacatgctcatagaaacagatcatcaacaaccttctccaacaacaaaccaacccaatgacccggttgatcatgacgaccccaaaacaaggattgtaaatgactgggctactatccatcagctcgtggcctctcaactcaatggccatgaagacacttcaaagcacttgtcctgcttcggtgacccaaacatggccttttgttcttctcctccttctaatgatgaggacttacagttgtcctatccatatctacgtccaggtagaatatcacagaatcaacccgaagtctacaacagcgagaacgatctatggagcttcacaaaatcgtcgccgtcgccgtcatcatcagacccattatgccacctgtcggtgtaa

(19) United States
(12) Patent Application Publication
(10) Pub. No.: US 2026/0337626 A1
(43) Pub. Date: Oct. 29, 2026

(54) METHODS AND COMPOSITIONS FOR MODIFYING ENDOCARP STRUCTURE IN PRUNUS PLANTS

(71) Applicant: Pairwise Plants Services, Inc., Durham, NC (US)

(72) Inventors: Brian Charles Wilding Crawford, Cary, NC (US); Marisa Miller, Durham, NC (US); Thomas J. Poorten, Durham, NC (US)

(21) Appl. No.: 19/655,036

(22) Filed: Apr. 22, 2026

Related U.S. Application Data
(60) Provisional application No. 63/792,497, filed on Apr. 22, 2025.

(51) Int. Cl. C12N 15/82; C07K 14/415

(52) U.S. Cl. CPC C12N 15/8262; C07K 14/415

(57) ABSTRACT

This invention relates to compositions and methods for producing Prunus plants exhibiting a phenotype of reduced endocarp lignification and reduced seediness or a reduced feel of seediness phenotypes through modification of endogenous NAC SECONDARY WALL THICKENING PROMOTING FACTOR1 (NST1) nucleic acids and endogenous NAC SECONDARY WALL THICKENING PROMOTING FACTOR3 (NST3) nucleic acids. The invention further relates to plants produced using the methods and compositions of the invention.

### STATEMENT OF PRIORITY

This application claims the benefit of U.S. Provisional Application No. 63/792,497, filed Apr. 22, 2025, the contents of which is incorporated by reference herein.

### STATEMENT REGARDING ELECTRONIC FILING OF A SEQUENCE LISTING

A Sequence Listing in XML format, entitled 1499-164WO_ST26.xml, 249,089 bytes in size, generated on Apr. 21, 2026, and filed herewith, is hereby incorporated by reference into the specification for its disclosures.

### FIELD OF THE INVENTION

This invention relates to compositions and methods for modifying endocarp structure in a Prunus plant through modification of endogenous NAC SECONDARY WALL THICKENING PROMOTING FACTOR1 (NST1) and endogenous NAC SECONDARY WALL THICKENING PROMOTING FACTOR3 (NST3) nucleic acids. The invention further relates to Prunus plants produced using the methods and compositions of the invention.

### BACKGROUND OF THE INVENTION

Fruit development normally depends on fertilization and the subsequent formation of the seed. Fruit can have hard seeds such as grapes or watermelons, or be drupes such as cherries, peaches or black raspberries. A drupe (or stone fruit) is an indehiscent fruit in which an outer fleshy part (exocarp, or skin; and mesocarp, or flesh) surrounds a single shell (the pit, stone, or pyrene) of hardened endocarp with a seed (kernel) inside. The endocarp is comprised of lignin to protect the seed. The endocarp develops from the innermost layer of the ovary. The endocarp is present in all fruit but in drupes is defined by the formation of the lignified pit which causes the sensation of ‘seediness’ in such plants as those from the genera of Prunus.

Canonically, seedless fruits can develop in one of two ways: parthenocarpy and stenospermocarpy. In parthenocarpy, the fruit develops without pollination or fertilization and is desirable in fruit crops that may be difficult to pollinate or fertilize, such as fig, tomato, and summer squash. In stenospermocarpy, pollination or fertilization triggers fruit development, but the ovules or embryos abort without producing mature seeds. In Prunus, no seedless varieties have been developed and commercially released, as it would require reduction or elimination of endocarp, as well as the enclosed seed in species with larger seeds.

The present invention overcomes the shortcomings in the art by providing improved methods and compositions for modifying plant fruit development and reduce seediness.

### SUMMARY OF THE INVENTION

One aspect of the invention provides a Prunus plant or plant part thereof comprising at least one mutation (e.g., one or more) in at least one endogenous NAC SECONDARY WALL THICKENING PROMOTING FACTOR1 (NST1) gene encoding a NST1 transcription factor polypeptide and at least one mutation in at least one endogenous NAC SECONDARY WALL THICKENING PROMOTING FACTOR3 (NST3) gene encoding a NST3 transcription factor polypeptide, wherein the at least one mutation in the at least one endogenous NST1 gene and/or the at least one mutation in the at least one endogenous NST3 gene is a null mutation or a dominant-negative mutation.

A second aspect of the invention provides a method for editing a specific site in the genome of a Prunus plant cell, the method comprising: cleaving, in a site-specific manner, a target site within an endogenous NAC SECONDARY WALL THICKENING PROMOTING FACTOR1 (NST1) gene in the Prunus plant cell and a target site within an endogenous NAC SECONDARY WALL THICKENING PROMOTING FACTOR3 (NST3) gene in the Prunus plant cell, wherein the target site is in a region of the NST1 gene having at least 80% sequence identity to any one of the nucleotide sequences of SEQ ID NOs: 72-76 or 84-87, and the target site is in a region of the NST3 gene having at least 80% sequence identity to any one of the nucleotide sequences of SEQ ID NOs: 91-95 or 102-105, thereby generating an edit in the endogenous NST1 gene and in the endogenous NST3 gene of the Prunus plant cell and the edit results in a null mutation or a dominant-negative mutation.

A third aspect of the invention provides a method for producing a Prunus plant or part thereof comprising at least one cell having a mutation in an endogenous NAC SECONDARY WALL THICKENING PROMOTING FACTOR1 (NST1) gene and a mutation in an endogenous NAC SECONDARY WALL THICKENING PROMOTING FACTOR1 (NST3) gene, the method comprising contacting a target site within the endogenous NST1 gene and a target site within the endogenous NST3 gene in the Prunus plant or part thereof with a nuclease comprising a cleavage domain and a nucleic acid binding domain, wherein the nucleic acid binding domain of the nuclease binds to a target site within the endogenous NST1 gene and a target site within the endogenous NST3 gene, and/or contacting a target site within the endogenous NST1 gene and a target site within the endogenous NST3 gene in the Prunus plant or part thereof with a first nuclease and a second nuclease each of which comprises a cleavage domain and a nucleic acid binding domain, wherein the nucleic acid binding domain of the first nuclease binds to a target site within the endogenous NST1 gene and the nucleic acid binding domain of the second nuclease binds to a target site within the endogenous NST3 gene, wherein the target site is in a region of the NST1 gene having at least 80% sequence identity to any one of the nucleotide sequences of SEQ ID NOs: 72-76 or 84-87, and the target site is in a region of the NST3 gene having at least 80% sequence identity to any one of the nucleotide sequences of SEQ ID NOs: 91-95 or 102-105, thereby producing a Prunus plant or part thereof comprising at least one cell having a mutation in the endogenous NST1 gene and in the endogenous NST3 gene, wherein the mutation in the endogenous NST1 gene and/or in the endogenous NST3 gene is a null mutation or a dominant-negative mutation.

Further provided are Prunus plants, plant cells, and plant parts produced by the methods of the invention and comprising in their genomes at least one mutation in at least one endogenous NST1 gene and at least one mutation in at least one endogenous NST3 gene, as well as polypeptides, polynucleotides, nucleic acid constructs, expression cassettes and vectors for making a plant, plant cell, and/or plant part of this invention.

These and other aspects of the invention are set forth in more detail in the description of the invention below.

### BRIEF DESCRIPTION OF THE DRAWINGS

FIG. 1 depicts exemplary expression constructs used in analyzing secondary cell wall biogenesis gene expression in cherry protoplasts.

FIG. 2 shows log 10 (weightFisher p-value) per contrast (vs pUC19) for up-regulated genes (Log 2 Fold Change (LFC)>1) in GO:0009834 (plant-type secondary cell wall biogenesis) across Blackberry, Cherry, and Peach. The dashed line marks the significance threshold. Overexpression of wild-type (WT) NST1 or WT NST3 produces strong enrichment of this pathway across all three species. Co-expression of wild-type with the dominant-negative (DN) also shows significant enrichment, while stop codon controls show no enrichment.

FIGS. 3A-3C show log 2 fold change in expression of secondary cell wall biogenesis genes (GO:0009834) vs. treatment for blackberry NST1 and NST3 (FIG. 3A), cherry NST1 and NST3 (FIG. 3B), and peach NST1 and NST3 (FIG. 3C) in cherry protoplasts. The treatment WT NST3+dominant-negative NST3 for blackberry and peach were not tested as plasmid construction failed. Blackberry NST3 containing a stop codon also was not tested, as plasmid construction failed.

FIGS. 4A-4D show log 2 fold change (FC) of secondary cell wall biogenesis genes (GO:0009834) for wild-type (WT) NST1 vs WT NST1+dominant-negative (DN) (both relative to pUC19) across Blackberry (FIG. 4A), Cherry (FIG. 4B), and Peach (FIG. 4C), as well as Cherry WT NST3 vs WT NST3+Dominant-negative (FIG. 4D). Strong positive Pearson correlations (r=0.786 Blackberry NST1, r=0.804 Cherry NST1, r=0.949 Peach NST1, r=0.655 Cherry NST3) with regression slopes below 1 indicate that co-expression of the dominant-negative attenuates but does not reverse wild-type-driven activation of GO:0009834 genes, consistent with the cancelling-out effect observed in the per-species analyses in cherry and blackberry.

### BRIEF DESCRIPTION OF THE SEQUENCES

SEQ ID NOs: 1-17 are exemplary Cas12a amino acid sequences useful with this invention.

SEQ ID NOs: 18-20 are exemplary Cas12a nucleotide sequences useful with this invention.

SEQ ID NOs: 21-22 are exemplary regulatory sequences encoding a promoter and intron.

SEQ ID NOs: 23-29 are exemplary cytosine deaminase sequences useful with this invention.

SEQ ID NOs: 30-40 are exemplary adenine deaminase amino acid sequences useful with this invention.

SEQ ID NO:41 is an exemplary uracil-DNA glycosylase inhibitor (UGI) sequence useful with this invention.

SEQ ID NOs: 42-44 provide example peptide tags and affinity polypeptides useful with this invention.

SEQ ID NOs: 45-55 provide example RNA recruiting motifs and corresponding affinity polypeptides useful with this invention.

SEQ ID NOs: 56-57 are exemplary Cas9 polypeptide sequences useful with this invention.

SEQ ID NOs: 58-68 are exemplary Cas9 polynucleotide sequences useful with this invention.

SEQ ID NOs: 69 and 81 are examples of Prunus NST1 genomic sequences.

SEQ ID NOs: 70 and 82 are examples of Prunus NST1 coding sequences, corresponding to genomic sequences of SEQ ID NOs: 69 and 81, respectively.

SEQ ID NOs: 71 and 83 are examples of Prunus NST1 polypeptide sequences.

SEQ ID NOs: 88 and 99 are examples of Prunus NST3 genomic sequences.

SEQ ID NOs: 89 and 100 are examples of Prunus NST3 coding sequences, corresponding to genomic sequences of SEQ ID NOs: 88 and 99, respectively.

SEQ ID NOs: 90 and 101 are examples of Prunus NST3 polypeptide sequences.

SEQ ID NOs: 72-76 and 84-87 are example regions or portions from Prunus NST1 polynucleotides.

SEQ ID NOs: 91-95 and 102-105 are example regions or portions from Prunus NST3 polynucleotides.

SEQ ID NOs: 77-80 are example regions or portions from Prunus NST1 polypeptides.

SEQ ID NOs: 80 and 96-98 are example regions or portions from Prunus NST3 polypeptides.

SEQ ID NO:106 is an example blackberry NST1 coding sequence.

SEQ ID NO:107 is an example of a blackberry NST1 polypeptide sequence.

SEQ ID NO:108 is an example blackberry NST3 coding sequence.

SEQ ID NO:109 is an example of a blackberry NST3 polypeptide sequence.

SEQ ID NOs: 110, 111, 112, and 113 are example spacer sequences for nucleic acid guides useful with this invention.

SEQ ID NOs: 114, 115, 118, and 119 are example mutated Prunus NST1 polypeptide sequences.

SEQ ID NOs: 116, 117, and 120 are example mutated Prunus NST3 polypeptide sequences.

SEQ ID NO:121 and SEQ ID NO:122 are example mutated blackberry NST1 polypeptide sequences.

SEQ ID NO:123 and SEQ ID NO:124 are example mutated blackberry NST3 polypeptide sequences.

SEQ ID NOs: 125, 126, 129, and 130 are example mutated Prunus NST1 coding sequences.

SEQ ID NOs: 127, 128, 131, and 132 are example mutated Prunus NST3 coding sequences.

SEQ ID NO:133 and SEQ ID NO:134 are example mutated blackberry NST1 coding sequences.

SEQ ID NO:135 and SEQ ID NO:136 are example mutated blackberry NST3 coding sequences.

SEQ ID NOs: 79 and 98 are example dimerization domains of Prunus NST1 and NST3 polypeptides, respectively.

### DETAILED DESCRIPTION

The present invention now will be described hereinafter with reference to the accompanying examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include λ as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In some embodiments, the reduction can result in no or essentially no (i.e., an insignificant amount, e.g., less than about 10% or even 5%) detectable activity or amount.

A “control plant” is typically the same plant as the edited plant, but the control plant has not been similarly edited and therefore is devoid of the mutation. A control plant may be an isogenic plant and/or a wild-type plant. Thus, a control plant can be the same breeding line, variety, or cultivar as the subject plant into which a mutation as described herein is introgressed, but the control breeding line, variety, or cultivar is free of the mutation. In some embodiments, a comparison between a plant of the invention and a control plant is made under the same growth conditions, e.g., the same environmental conditions (soil, hydration, light, heat, nutrients, and the like).

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

As used herein, the term “heterologous” refers to a nucleotide/polypeptide that originates from a foreign species, or, if from the same species, is substantially modified from its native form in composition and/or genomic locus by deliberate human intervention. A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild-type” nucleic acid, nucleotide sequence, polypeptide or amino acid sequence refers to a naturally occurring endogenous nucleic acid, nucleotide sequence, polypeptide or amino acid sequence. In some contexts, a “wild-type” nucleic acid is a nucleic acid that is not edited as described herein and can differ from an “endogenous” gene that may be edited as described herein (e.g., a mutated endogenous gene). In some contexts, a “wild-type” nucleic acid (e.g., unedited) may be heterologous to the organism in which the wild-type nucleic acid is found (e.g., a transgenic organism). As an example, a “wild-type endogenous NST1 gene” is a NST1 gene that is naturally occurring in or endogenous to the reference organism, e.g., a plant, and may be subject to modification as described herein, after which, such a modified endogenous gene is no longer wild-type. Similarly, a “wild-type endogenous NST3 gene” is a NST3 gene that is naturally occurring in or endogenous to the reference organism, e.g., a plant, and may be subject to modification as described herein, after which, such a modified endogenous gene is no longer wild-type.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus, e.g., alternative forms of a gene that arise by mutation and are found at the same place on a chromosome.

A “recessive allele” is an allele in a gene that produces a phenotype when homozygous but the phenotype is not observable when the locus is heterozygous.

A “dominant allele” is an allele in a gene that produces a phenotype whether its paired allele is identical or different.

A “null mutation” is a mutation that results in a complete lack of production of the corresponding protein or produces a protein that is non-functional.

A “knockout mutation” is a mutation that results in a non-functional protein, but which may have a detectable transcript or protein.

A “recessive mutation” is a mutation in a gene that produces a phenotype when homozygous but the phenotype is not observable when the locus is heterozygous.

A “dominant mutation” is a mutation in a gene that produces a mutant phenotype in the presence of a non-mutated copy of the gene. A dominant mutation may be a loss or a gain of function mutation, a hypomorphic mutation, a hypermorphic mutation or a weak loss of function or a weak gain of function.

A “dominant negative mutation” is a mutation that produces an altered gene product (e.g., having an aberrant function relative to wild-type), which gene product adversely affects the function of the wild-type allele or gene product. For example, a “dominant negative mutation” may block a function of the wild-type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wild-type gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

A “gain-of-function” allele or mutation is a mutation that confers a new function on the encoded gene product and/or confers a new gene expression pattern. In some embodiments, a gain-of-function mutation may be dominant or semi-dominant.

As used herein, a “non-natural mutation” refers to a mutation that is generated through human intervention and differs from mutations found in the same gene that have occurred in nature (e.g., occurred naturally and not as a result of a modification made by a human). As an example, a non-natural mutation may be a mutation generated using a gene editing system such as editing systems comprising a nuclease (e.g., a zinc finger nuclease, transcription activator-like effector nuclease (TALEN), endonuclease (e.g., Fok1) or a CRISPR-Cas effector protein).

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype. In some embodiments of this invention, the phrase “desired allele,” “target allele” or “allele of interest” refers to an allele(s) that is associated with increased yield under non-water stress conditions in a plant relative to a control plant not having the target allele or alleles.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in Techniques Et Utilisations Des Marqueurs Moleculaires Les Colloques, Vol. 72, pp. 45-56 (1995); and Openshaw et al., Marker-assisted Selection in Backcross Breeding, in Proceedings of the Symposium “Analysis of Molecular Marker Data,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from it's parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

A plant in which at least one (e.g., one or more, e.g., 1, 2, 3, or 4, or more) endogenous NST1 gene and at least one endogenous NST3 gene are modified as described herein (e.g., comprises a modification as described herein) may have improved or increased yield traits as compared to a plant that does not comprise (is devoid of) the modification in the at least one endogenous NST1 gene and in the at least one endogenous NST3 gene. As used herein, “improved yield traits” refers to any plant trait associated with growth, for example, biomass, yield, nitrogen use efficiency (NUE), inflorescence size/weight, fruit yield, fruit quality, fruit size, seed size (e.g., seed area, seed size), seed number, foliar tissue weight, nodulation number, nodulation mass, nodulation activity, number of seed heads, number of tillers, number of branches, number of flowers, number of tubers, tuber mass, bulb mass, number of seeds, total seed mass, rate of leaf emergence, rate of tiller/branch emergence, rate of seedling emergence, length of roots, number of roots, root angle, size and/or weight of root mass, or any combination thereof. In some aspects, “improved yield traits” may include, but are not limited to, increased inflorescence production, increased fruit production (e.g., increased number, weight and/or size of fruit; e.g., increased number, weight, and/or length of ears for, e.g., maize), increased fruit quality, increased number, size and/or weight of roots, and/or root angle, increased meristem size, increased seed size (e.g., seed area and/or seed weight), increased biomass, increased leaf size, increased nitrogen use efficiency, increased height, increased internode number and/or increased internode length as compared to a control plant or part thereof (e.g., a plant that does not comprise a mutated endogenous NST1 nucleic acid and/or a mutated and endogenous NST3 nucleic acid as described herein). In some aspects, improved yield traits can be expressed as quantity of grain/seed produced per area of land (e.g., bushels per acre of land). In some embodiments, a plant or part thereof comprising the at least one mutation in an endogenous NST1 gene and at least one mutation in an endogenous NST3 gene may exhibit a phenotype of improved yield traits, optionally exhibiting an increased yield (bu/acre), increased kernel size, increased kernel weight, and/or increased ear length, optionally without substantially increasing ear width, as compared to a plant that is devoid of the at least one mutation.

As used herein a “control plant” means a plant that does not contain an edited NST1 gene and/or edited NST3 gene as described herein. A control plant is used to identify and select a plant edited as described herein and that exhibits reduced endocarp lignification and reduced seediness or a reduced feel of seediness phenotypes as compared to the control plant. A suitable control plant can be a plant of the parental line used to generate a plant comprising a mutated NST1 gene and mutated NST3 gene, for example, a wild-type plant devoid of an edit in an endogenous NST1 gene and/or endogenous NST3 gene as described herein. A suitable control plant can also be a plant that contains recombinant nucleic acids that impart other traits, for example, a transgenic plant having enhanced herbicide tolerance. A suitable control plant can in some cases be a progeny of a heterozygous or hemizygous transgenic plant line that is devoid of the mutated NST1 gene and/or mutated NST3 gene as described herein, known as a negative segregant, or a negative isogenic line.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing northern analysis, RT-PCR, microarray gene expression assays, or reporter gene expression systems, or by agricultural observations such as hyperosmotic stress tolerance or yield. However, any technique can be used to measure the amount of, the comparative level of, or the difference in any selected chemical compound or macromolecule in the transgenic plants.

As used herein an “enhanced trait” means a characteristic of a plant resulting from mutations in a NST1 gene and in a NST3 gene as described herein. Such traits include, but are not limited to, an enhanced agronomic trait characterized by enhanced plant morphology, physiology, growth and development, yield, nutritional enhancement, disease or pest resistance, or environmental or chemical tolerance. In some embodiments, an enhanced trait/altered phenotype may be, for example, decreased days from planting to maturity, reduced endocarp lignification, reduced endocarp lignification and reduced pit or seed hardness, reduced seediness, reduced feel of seediness, increased number of root branches, increased total root length, drought tolerance, increased water use efficiency, cold tolerance, increased nitrogen use efficiency, and/or increased yield. In some embodiments, a trait is increased yield under nonstress conditions or increased yield under environmental stress conditions. Stress conditions can include both biotic and abiotic stress, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density.

Also used herein, the term “trait modification” encompasses altering the naturally occurring trait by producing a detectable difference in a characteristic in a plant comprising a mutation in an endogenous NST1 gene and mutation in an endogenous NST3 gene as described herein relative to a plant not comprising the mutation, such as a wild-type plant, or a negative segregant. In some cases, the trait modification can be evaluated quantitatively. For example, trait modification can entail an increase or decrease in an observed trait characteristic or phenotype as compared to a control plant. It is known that there can be natural variations in a modified trait. Therefore, the trait modification observed can entail a change of the normal distribution and magnitude of the trait characteristics or phenotype in the plants as compared to a control plant.

The present disclosure relates to a plant with improved economically relevant characteristics, more specifically (1) reduced endocarp lignification, (2) reduced endocarp lignification and reduced pit or seed hardness, and/or (3) reduced seediness or a reduced feel of seediness phenotypes. More specifically the present disclosure relates to a plant comprising a mutation(s) in a NST1 gene and a NST3 gene as described herein, wherein the plant exhibits reduced endocarp lignification, reduced endocarp lignification and reduced pit or seed hardness, and/or reduced seediness or a reduced feel of seediness phenotypes as compared to a control plant devoid of said mutation(s). In some embodiments, a plant of the present disclosure further exhibits an improved trait that is related to yield, including but not limited to increased nitrogen use efficiency, increased nitrogen stress tolerance, increased water use efficiency and/or increased drought tolerance, as defined and discussed infra.

Yield can be defined as the measurable product of economic value from a crop. Yield can be defined in the scope of quantity and/or quality. Yield can be directly dependent on several factors, for example, the number and size of organs (e.g., number of flowers), plant architecture (such as the number of branches, plant biomass, e.g., increased root biomass, steeper root angle and/or longer roots, and the like), flowering time and duration. Root architecture and development, photosynthetic efficiency, nutrient uptake, stress tolerance, early vigor, delayed senescence and functional stay green phenotypes may be factors in determining yield. Optimizing the above-mentioned factors can therefore contribute to increasing crop yield.

Reference herein to an increase/improvement in yield-related traits can also be taken to mean an increase in biomass (weight) of one or more parts of a plant, which can include above ground and/or below ground (harvestable) plant parts. The term “yield” of a plant can relate to vegetative biomass (root and/or shoot biomass), to reproductive organs, and/or to propagules (such as seeds) of that plant.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part (e.g., fruits); or (ii) increased early vigor, defined herein as an improved seedling aboveground area approximately three weeks post-germination.

“Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor, for example, can be a combination of the ability of seeds to germinate and emerge after planting and the ability of the young plants to grow and develop after emergence. Plants having early vigor also show increased seedling survival and better establishment of the crop, which often results in highly uniform fields with the majority of the plants reaching the various stages of development at substantially the same time, which often results in increased yield. Therefore, early vigor can be determined by measuring various factors, such as kernel weight, percentage germination, percentage emergence, seedling growth, seedling height, root length, root and shoot biomass, canopy size and color and others.

Further, increased yield can also manifest as increased total fruit yield, which may result from one or more of an increase in fruit biomass (fruit weight) due to an increase in the fruit weight on a per plant and/or on an individual fruit basis an increased number of, for example, flowers/panicles per plant; an increased number of flowers (“florets”) per panicle/plant; an increased number of fruits; increased fruit size (length, width, area, perimeter, and/or weight). In one embodiment, increased yield can be increased fruit yield, for example, increased fruit weight; increased number of fruits; and/or increased harvest index.

Increased yield can also result in modified architecture or can occur because of modified plant architecture.

Increased yield can also manifest as increased harvest index, which is expressed as a ratio of the yield of harvestable parts, such as fruit, over the total biomass.

The disclosure also extends to harvestable parts of a plant such as, but not limited to, seeds, leaves, fruits, flowers, bolls, pods, siliques, nuts, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example fruit, or weight of fruit, per acre, pounds per acre, bushels per acre, tons per acre, tons per acre, kilo per hectare.

As used herein “nitrogen use efficiency” refers to the processes which lead to an increase in the plant's yield, biomass, vigor, and growth rate per nitrogen unit applied. The processes can include the uptake, assimilation, accumulation, signaling, sensing, retranslocation (within the plant) and use of nitrogen by the plant.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance and can also improve crop quality and biochemical constituents of the seed such as protein yield and oil yield. The terms “increased nitrogen use efficiency,” “enhanced nitrogen use efficiency,” and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Typically, plants are more susceptible to drought during flowering and seed development (the reproductive stages), as plant's resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant in the short term. The terms “increased water use efficiency,” “enhanced water use efficiency,” and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein, “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein, “water deficit” refers to the conditions or environments that provide less than optimal amounts of water needed for adequate/successful growth and development of plants.

As used herein, “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the World Intellectual Property Organization (WIPO) Standard ST.26. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950 or 1000 or more nucleotides or any range or value therein) relative to a reference nucleic acid and that comprises, consists essentially of and/or consists of a nucleotide sequence of contiguous nucleotides identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a portion of a wild-type CRISPR-Cas repeat sequence (e.g., a wild-type CRISR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

In some embodiments, a nucleic acid fragment or portion may comprise, consist essentially of or consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 90, 95, 100, 101, 102, 103, 104, 105, 110, 111, 112, 113, 114, 115, 120, 121, 122, 123, 124, 125, 126, 127, 128, 129, 130, 135, 140, 141, 142, 143, 144, 145, 150, 151, 152, 153, 154, 155, 160, 165, 170, 175, 176, 177, 178, 179, 180, 185, 190, 191, 192, 193, 194, 195, 200, 205, 210, 215, 216, 217, 218, 219, 220, 221, 222, 223, 224, 225, 230, 235, 240, 245, 250, 255, 256, 257, 258, 259, 260, 265, 270, 271, 272, 273, 274, 275, 280, 285, 290, 295, 300, 305, 310, 320, 330, 335, 336, 337, 338, 339, 340, 350, 360, 370, 380, 390, 395, 400, 410, 415, 420, 425, 430, 435, 440, 445, 450, 500, 550, 600, 660, 700, 750, 800, 850, 900, 950, 1000, 1050, 1100, 1150, 1200, 1250, 1300, 1350, 1400, 1450, or 1500, or more consecutive nucleotides or any range or value therein, of a NST1 polynucleotide (e.g., genomic DNA or coding sequence) or a NST3 polynucleotide (e.g., genomic DNA or coding sequence), optionally a fragment of a NST1 polynucleotide or a fragment of a NST3 polynucleotide may be about 20 nucleotides to about 120 nucleotides, about 20 nucleotides to about 250 nucleotides, about 20 nucleotides to about 350 nucleotides, about 100 nucleotides to about 250 nucleotides, about 100 nucleotides to about 350 nucleotides, about 150 nucleotides to about 400 nucleotides, about 60 nucleotides to about 300 nucleotides, about 60 nucleotides to about 550 nucleotides, about 60 nucleotides to about 1000 nucleotides, e.g., about 60, 80, 100, 120, 140, 160, 180, 200, 210, 220, 240, 260, 280, 300, or 350 consecutive nucleotides to about 400, 425, 450, 475, 500, 525, 550, 575, 600, 625, 650, 675, 700, 725, 750, 775, 800, 825, 850, 875, 900, 925, 950, 975, or 1000 or more consecutive nucleotides (e.g., consecutive nucleotides of any one of SEQ ID NOs: 69, 70, 81, 82, 88, 89, 99, or 100, see, e.g., SEQ ID NOs: 72-76, 84-87, 91-95 or 102-105).

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 5, 6, 7, 8, 9, 10, 11, 12, 13, 14, 15, 20, 21, 22, 23, 24, 25, 26, 27, 28, 29, 30, 35, 40, 45, 46, 47, 48, 49, 50, 55, 60, 65, 70, 75, 80, 85, 90, 95, 100, 125, 150, 175, 200, 225, 250, 300, 350, 400 or more consecutive amino acids of a reference polypeptide. In some embodiments, a fragment of a NST1 polypeptide or a fragment of a NST3 polypeptide comprises, consists essentially of or consists of at least about 5, 6, 7, 8, 9, 10, 11, 12, 13, 14, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, or 75 or more consecutive amino acids (e.g., a fragment or portion of SEQ ID NOs: 71, 83, 90, or 101 (e.g., SEQ ID NOs: 77-80 or 96-98).

In some embodiments, a “portion” may be related to the number of amino acids that are deleted from a polypeptide. Thus, for example, a deleted “portion” of a NST1 polypeptide or a NST3 polypeptide may comprise at least one amino acid residue (e.g., at least 1, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more consecutive amino acid residues) deleted from the amino acid sequence of SEQ ID NOs: 71, 83, 90, or 101 (e.g., SEQ ID NOs: 77-80 or 96-98), (or from a sequence having at least 80%, 85%, 90% or 95% sequence identity (e.g., at least 95, 96, 97, 98, 99 or 100% identity) (e.g., at least 95% sequence identity or 100% sequence identity) to the amino acid sequence of SEQ ID NOs: 71, 83, 90, and/or 101) (e.g., a deletion of about 1, 2, 3, 4, 5, 6, 7, 8, 9, or 10 to about 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400 or more residues or any range or value therein).

A “region” of a polynucleotide or a polypeptide refers to a portion of consecutive nucleotides or consecutive amino acid residues of that polynucleotide or a polypeptide, respectively. For example, a region of a NST1 polynucleotide sequence may include, but is not limited to, to a sequence having at least 80% sequence identity to any one of the nucleic acid sequences of SEQ ID NOs: 72-76 and/or 84-87. A region of a NST3 polynucleotide sequence may include, but is not limited to, to a sequence having at least 80% sequence identity to any one of the nucleic acid sequences of SEQ ID NOs: 91-95 and/or 102-105. In some embodiments, a region may be a target region or target site for modification in the NST1 polynucleotide and/or NST3 polynucleotide.

In some embodiments, a “sequence-specific nucleic acid binding domain” (e.g., sequence-specific DNA binding domain) may bind to a NST1 gene (e.g., SEQ ID NOs: 69, 70, 81, or 82) and/or to one or more fragments, portions, or regions of a NST1 nucleic acid (e.g., SEQ ID NOs: 72-76 and/or 84-87) and/or a NST3 gene (e.g., SEQ ID NOs: 88, 89, 99, or 100) and/or to one or more fragments, portions, or regions of a NST3 nucleic acid (e.g., SEQ ID NOs: 91-95 and/or 102-105).

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncated polypeptide can be the result of a deletion of the corresponding 5′ end or 3′ end of the gene encoding the polypeptide. A frameshift mutation can occur when deletions or insertions of one or more base pairs are introduced into a gene, optionally resulting in an out-of-frame mutation or an in-frame mutation. Frameshift mutations in a gene can result in the production of a polypeptide that is longer, shorter or the same length as the wild-type polypeptide depending on when the first stop codon occurs following the mutated region of the gene. As an example, an out-of-frame mutation that produces a premature stop codon can produce a polypeptide that is shorter than the wild-type polypeptide, or, in some embodiments, the polypeptide may be absent/undetectable. A DNA inversion is the result of a rotation of a genetic fragment within a region of a chromosome.

The term “complementary” or “complementarity,” as used herein, refers to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity; e.g., substantial complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide. In regard to a NST1 gene, a sequence may have at least 80% sequence identity to the nucleotide sequence of SEQ ID NOs: 69, 70, 81, and/or 82. In some embodiments, a NST1 gene may have at least 85% sequence identity to the nucleotide sequence of SEQ ID NOs: 69, 70, 81, and/or 82. In some embodiments, a NST1 gene may have at least 90% sequence identity to the nucleotide sequence of SEQ ID NOs: 69, 70, 81, and/or 82. In some embodiments, a NST1 gene may have at least 95% sequence identity (e.g., at least about 95%, 95.5%, 96%, 96.5%, 97%, 97.5%, 98%, 98.5%, 99%, 99.5%, or 100% sequence identity) to the nucleotide sequence of SEQ ID NOs: 69, 70, 81, and/or 82, optionally wherein the NST1 gene may have 100% sequence identity to the nucleotide sequence of SEQ ID NOs: 69, 70, 81, and/or 82. In regard to a NST3 gene, a sequence may have at least 80% sequence identity to the nucleotide sequence of SEQ ID NOs: 88, 89, 99, and/or 100. In some embodiments, a NST3 gene may have at least 85% sequence identity to the nucleotide sequence of SEQ ID NOs: 88, 89, 99, and/or 100. In some embodiments, a NST3 gene may have at least 90% sequence identity to the nucleotide sequence of SEQ ID NOs: 88, 89, 99, and/or 100. In some embodiments, a NST3 gene may have at least 95% sequence identity (e.g., at least about 95.5%, 96%, 96.5%, 97%, 97.5%, 98%, 98.5%, 99%, 99.5%, or 100% sequence identity) to the nucleotide sequence of SEQ ID NOs: 88, 89, 99, and/or 100, optionally wherein the NST3 gene may have 100% sequence identity to the nucleotide sequence of SEQ ID NOs: 88, 89, 99, and/or 100. A NST1 polypeptide as described herein may have at least 80% sequence identity to the polypeptide sequence of SEQ ID NO:71 and/or SEQ ID NO:83. In some embodiments, a NST1 polypeptide may have at least 85% sequence identity to the polypeptide sequence of SEQ ID NO:71 and/or SEQ ID NO:83. In some embodiments, a NST1 polypeptide may have at least 90% sequence identity to the polypeptide sequence of SEQ ID NO:71 and/or SEQ ID NO:83. In some embodiments, a NST1 polypeptide may have at least 95% sequence identity (e.g., at least about 95.5%, 96%, 96.5%, 97%, 97.5%, 98%, 98.5%, 99%, 99.5%, or 100% sequence identity) to the polypeptide sequence of SEQ ID NO: 71 and/or SEQ ID NO:83, optionally wherein the NST1 polypeptide may have 100% sequence identity to the polypeptide sequence of any one of SEQ ID NO:71 and/or SEQ ID NO: 83. A NST3 polypeptide as described herein may have at least 80% sequence identity to the polypeptide sequence of SEQ ID NO:90 and/or SEQ ID NO:101. In some embodiments, a NST3 polypeptide may have at least 85% sequence identity to the polypeptide sequence of SEQ ID NO:90 and/or SEQ ID NO:101. In some embodiments, a NST3 polypeptide may have at least 90% sequence identity to the polypeptide sequence of SEQ ID NO:90 and/or SEQ ID NO:101. In some embodiments, a NST3 polypeptide may have at least 95% sequence identity (e.g., at least about 95.5%, 96%, 96.5%, 97%, 97.5%, 98%, 98.5%, 99%, 99.5%, or 100% sequence identity) to the polypeptide sequence of SEQ ID NO:90 and/or SEQ ID NO:101, optionally wherein the NST3 polypeptide may have 100% sequence identity to the polypeptide sequence of SEQ ID NO:90 and/or SEQ ID NO:101. With regard to regions or portions of a NST1 gene, the region or portion may have at least 80% sequence identity to the nucleotide sequence of any one of SEQ ID NOs: 72-76 and/or 84-87. In some embodiments, a region or portion of a NST1 gene may have at least 85% sequence identity to the nucleotide sequence of any one of SEQ ID NOs: 72-76 and/or 84-87. In some embodiments, a region or portion of a NST1 gene may have at least 90% sequence identity to the nucleotide sequence of any one of SEQ ID NOs: 72-76 and/or 84-87. In some embodiments, a region or portion of a NST1 gene may have at least 95% sequence identity (e.g., at least about 95.5%, 96%, 96.5%, 97%, 97.5%, 98%, 98.5%, 99%, 99.5%, or 100% sequence identity) to the nucleotide sequence of any one of SEQ ID NOs: 72-76 and/or 84-87, optionally wherein the region or portion of a NST1 gene may have 100% sequence identity to the nucleotide sequence of any one of SEQ ID NOs: 72-76 and/or 84-87. With regard to regions or portions of a NST3 gene, the region or portion may have at least 80% sequence identity to the nucleotide sequence of any one of SEQ ID NOs: 91-95 and/or 102-105. In some embodiments, a region or portion of a NST3 gene may have at least 85% sequence identity to the nucleotide sequence of any one of SEQ ID NOs: 91-95 and/or 102-105. In some embodiments, a region or portion of a NST3 gene may have at least 90% sequence identity to the nucleotide sequence of any one of SEQ ID NOs: 91-95 and/or 102-105. In some embodiments, a region or portion of a NST3 gene may have at least 95% sequence identity (e.g., at least about 95.5%, 96%, 96.5%, 97%, 97.5%, 98%, 98.5%, 99%, 99.5%, or 100% sequence identity) to the nucleotide sequence of any one of SEQ ID NOs: 91-95 and/or 102-105, optionally wherein the region or portion of a NST3 gene may have 100% sequence identity to the nucleotide sequence of any one of SEQ ID NOs: 91-95 and/or 102-105. With regard to regions or portions of a NST1 polypeptide, the region or portion may have at least 80% sequence identity to the polypeptide sequence of any one of SEQ ID NOs: 77-80, optionally to the polypeptide sequence of any one of SEQ ID NOs: 77-80. In some embodiments, a region or portion of a NST1 polypeptide may have at least 85% sequence identity to the amino acid sequence of any one of SEQ ID NOs: 77-80. In some embodiments, a region or portion of a NST1 polypeptide may have at least 90% sequence identity to the amino acid sequence of any one of SEQ ID NOs: 77-80. In some embodiments, a region or portion of a NST1 polypeptide may have at least 95% sequence identity (e.g., at least about 95.5%, 96%, 96.5%, 97%, 97.5%, 98%, 98.5%, 99%, 99.5%, or 100% sequence identity) to the amino acid sequence of any one of SEQ ID NOs: 77-80. In some embodiments, a region or portion of a NST1 polypeptide may have at least 100% sequence identity to the amino acid sequence of any one of SEQ ID NOs: 77-80. With regard to regions or portions of a NST3 polypeptide, the region or portion may have at least 80% sequence identity to the polypeptide sequence of any one of SEQ ID NOs: 80 and/or 96-98. In some embodiments, a region or portion of a NST3 polypeptide may have at least 85% sequence identity to the amino acid sequence of any one of SEQ ID NOs: 80 and/or 96-98. In some embodiments, a region or portion of a NST3 polypeptide may have at least 90% sequence identity to the amino acid sequence of any one of SEQ ID NOs: 80 and/or 96-98. In some embodiments, a region or portion of a NST3 polypeptide may have at least 95% sequence identity (e.g., at least about 95.5%, 96%, 96.5%, 97%, 97.5%, 98%, 98.5%, 99%, 99.5%, or 100% sequence identity) to the amino acid sequence of any one of SEQ ID NOs: 80 and/or 96-98. In some embodiments, a region or portion of a NST3 polypeptide may have at least 100% sequence identity to the amino acid sequence of any one of SEQ ID NOs: 80 and/or 96-98.

In some embodiments, an NST1 gene having a mutation as described herein comprises a sequence having at least 90% identity to any one of SEQ ID NOs: 125, 126, 129 and 130, and/or an NST3 gene having a mutation as described herein comprises a sequence having at least 90% identity to any one of SEQ ID NOs: 127, 128, 131, and 132. In some embodiments, an NST1 polypeptide having a mutation as described herein comprises a sequence having at least 90% identity to any one of SEQ ID NOs: 114, 115, 118, and 119, and/or an NST3 polypeptide having a mutation as described herein comprises a sequence having at least 90% identity to any one of SEQ ID NOs: 116, 117, and 120.

As used herein, the phrase “substantially identical,” or “substantial identity” in the context of two nucleic acid molecules, nucleotide sequences or polypeptide sequences, refers to two or more sequences or subsequences that have at least about 95.5%, 96%, 96.5%, 97%, 97.5%, 98%, 98.5%, 99%, 99.5%, or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more nucleotides in length, and any range therein, up to the full length of the sequence. In some embodiments, nucleotide sequences can be substantially identical over at least about 20 consecutive nucleotides (e.g., about 20, 21, 22, 23, 24, 25, 26, 27, 28, 29, 30, 31, 32, 33, 34, 35, 36, 37, 38, 39, 40, 50, 60, 70, 80, 90, 100, 200, 300, 400, 500, 600, 700, 800, 900, 1000, 1100, 1200, 1300, 1400, or 1500, or more nucleotides). In some embodiments, two or more NST1 genes or two or more NST3 may be substantially identical to one another over at least about 30 or more consecutive nucleotides (e.g., 30, 31, 32, 33, 34, 35, 36, 37, 38, 39, 40, 41, 42, 43, 44, 45, 46, 47, 48, 49, 50, 51, 52, 53, 54, 54, 56, 57, 58, 59, 60, 65, 70, 75, 80, 85, 90, 95, 100, 110, 120, 130, 140, 150, 160, 170, 180, 200, 220, 240, 260, 280, 300, 320, 340, 360, 380, 400, 420, 440, 460, 480, 500, 520, 540, 560, 580, 600, or more consecutive nucleotides) of any one SEQ ID NOs: 69, 70, 81, 82, 88, 89, 99, or 100 (see, e.g., SEQ ID NOs: 72-76, 84-87, 91-95 and/or 102-105).

In some embodiments of the invention, the substantial identity exists over a region of consecutive amino acid residues of a polypeptide of the invention that is about 3 amino acid residues to about 20 amino acid residues, about 5 amino acid residues to about 10 amino acid residues, about 5 amino acid residues to about 55 amino acid residues, about 5 amino acid residues to about 25 amino acid residues, about 7 amino acid residues to about 30 amino acid residues, about 10 amino acid residues to about 25 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8, 9, 10, 11, 12, 13, 14, or more consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 275, 300, 325, 350, 400, or more amino acids in length or more consecutive amino acid residues). In some embodiments, two or more NST1 polypeptides or two or more NST3 polypeptides may be substantially identical to one another over at least about 10 to about 150 (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145 or 150 residues or more) consecutive amino acid residues of SEQ ID NOs: 71, 83, 90, and/or 101 (see, e.g., SEQ ID NOs: 77-80 and/or 96-98). In some embodiments, a substantially identical nucleotide or protein sequence may perform substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5 °C lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42 °C, with the hybridization being carried out overnight. An example of highly stringent wash conditions is 0.1 5M NaCl at 72 °C for about 15 minutes. An example of stringent wash conditions is a 0.2×SSC wash at 65 °C for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45 °C for 15 minutes. An example of a low stringency wash for a duplex of, e.g., more than 100 nucleotides, is 4-6×SSC at 40 °C for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30 °C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The polynucleotide and/or recombinant nucleic acid constructs of this invention can be optimized for expression. In some embodiments, the polynucleotides, nucleic acid constructs, expression cassettes, and/or vectors of the invention (e.g., comprising/encoding an RNA-guided DNA nuclease (e.g., a CRISPR-Cas effector protein (e.g., a Type V CRISPR-Cas effector protein)), a reverse transcriptase, a flap endonuclease, a 5′-3′ exonuclease, and the like) are optimized for expression in an organism (e.g., in a particular species), optionally an animal, a plant, a fungus, an archaeon, or a bacterium. In some embodiments, the optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%) identity or more to the nucleic acid constructs, polynucleotides, expression cassettes, and/or vectors that have not been optimized for expression.

Any nucleotide sequence, polynucleotide and/or recombinant nucleic acid construct of this invention can be optimized for expression in any organism of interest. Optimization for expression is well known in the art and involves, for example, modification of a nucleotide sequence for codon usage bias using species specific codon usage tables. Also relevant for optimization of expression is the editing efficiency of the target gene. Thus, the ability to generate a specific edit with a frequency that produces a sufficient number of edited alleles to enable selection of a commercially relevant edited allele of a target gene is a factor to be considered when optimizing for expression. In some embodiments, a polynucleotide, nucleic acid construct, expression cassette, and/or vector of the present invention (e.g., that comprises/encodes a polypeptide of the present invention (e.g., an engineered protein), a nucleic acid binding polypeptide (e.g., a DNA binding polypeptide such as a sequence-specific DNA binding domain from a polynucleotide-guided endonuclease, a zinc finger nuclease, a transcription activator-like effector nuclease (TALEN), an Argonaute protein, and/or a CRISPR-Cas effector protein), a guide nucleic acid, a cytosine deaminase and/or adenine deaminase) may be optimized for expression in an organism (e.g., an animal such as a human, a plant, a fungus, an archaeon, or a bacterium). In some embodiments, the optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9% or 100%) identity or more to the reference nucleic acid constructs, polynucleotides, expression cassettes, and/or vectors but which have not been optimized for expression.

To modify a nucleotide sequence for codon usage, codon usage tables are generated based on a sequence analysis of the most highly expressed genes for a organism/species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. As is understood in the art, codon optimization of a nucleotide sequence results in a nucleotide sequence having less than 100% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or 99.9%, and any range or value therein) to the native nucleotide sequence but which still encodes a polypeptide having the same function as that encoded by the original, native nucleotide sequence. Thus, in some embodiments of the invention, the polynucleotides, nucleic acid constructs, expression cassettes, and/or vectors of the invention (e.g., comprising/encoding a polypeptide, fusion protein, complex of the invention, e.g., a modified CRISPR-Cas nuclease) are codon optimized for expression in a particular species of interest, e.g., a particular plant species, a particular bacterial species, a particular animal species, and the like. In some embodiments, the codon optimized nucleic acid constructs, polynucleotides, expression cassettes, and/or vectors of the invention have about 70% to about 99.9% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9% or 100%) identity or more to the polynucleotides, nucleic acid constructs, expression cassettes, and/or vectors of the invention that have not been codon optimized.

A polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron) (see, e.g., SEQ ID NO:21 and SEQ ID NO: 22).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a DNA binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140, 150 or more amino acids in length). In some embodiments, a peptide linker may be a Gly-Ser (GS) linker.

In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter.

The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RuBisCO small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. (2005) Plant Cell Rep. 23:727-735; Li et al. (2007) Gene 403:132-142; Li et al. (2010) Mol Biol. Rep. 37:1143-1154). PrbcS1 and Pactin are constitutive promoters and Pnr and Pdca1 are inducible promoters. Pnr is induced by nitrate and repressed by ammonium (Li et al. (2007) Gene 403:132-142) and Pdca1 is induced by salt (Li et al. (2010) Mol Biol. Rep. 37:1143-1154). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, Ubi promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al. (1991) Plant Science 79:87-94), maize (Christensen et al. (1989) Plant Molec. Biol. 12:619-632), and Arabidopsis (Norris et al. (1993) Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. ((1991) Mol. Gen. Genet. 231:150-160) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula (1989) Plant Molec. Biol. 12:579-589). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf, or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond ((1991) FEBS 290:103-106; EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. (2015) Plant Biotechnol. Reports 9 (5): 297-306), ZmSTK2_USP from maize (Wang et al. (2017) Genome 60 (6): 485-495), LAT52 and LAT59 from tomato (Twell et al. (1990) Development 109 (3): 705-713), Zm13 (U.S. Pat. No. 10,421,972), PLA₂-δ promoter from Arabidopsis (U.S. Pat. No. 7,141,424), and/or the ZmC5 promoter from maize (International PCT Publication No. WO1999/042587).

Additional examples of plant tissue-specific/tissue preferred promoters include, but are not limited to, the root hair-specific cis-elements (RHEs) (Kim et al. (2006) The Plant Cell 18:2958-2970), the root-specific promoters RCc3 (Jeong et al. (2010) Plant Physiol. 153:185-197) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology 37 (8): 1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989) supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific nucleic acid binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific nucleic acid binding domain (e.g., sequence-specific DNA binding domain), a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, a polynucleotide encoding a sequence specific nucleic acid binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific DNA binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific DNA binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered, or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited to, a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g., higher plant, mammalian, yeast, or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific DNA binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific DNA binding protein, the reverse transcriptase and the deaminase are expressed and the sequence-specific DNA binding protein binds to the target nucleic acid, and the reverse transcriptase and/or deaminase may be fused to either the sequence-specific DNA binding protein or recruited to the sequence-specific DNA binding protein (via, for example, a peptide tag fused to the sequence-specific DNA binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment. As described herein, a target nucleic acid may also be contacted with an RNAi molecule designed for reducing expression of the target gene, e.g., NST1 and NST3.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

The term “regulating” as used in the context of a transcription factor “regulating” a phenotype, for example, a response to illumination (e.g., a light response, e.g., a shade avoidance response), means the ability of the transcription factor to affect the expression of a gene or genes such that a phenotype, for instance, a response to illumination, is modified.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. (2013) Nat. Biotechnol. 31:233-239; Ran et al. (2013) Nature Protocols 8:2281-2308). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska ((2002) Cell. Mol. Biol. Lett. 7:849-858).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior of the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

The present invention is directed to Prunus plants and the production of Prunus plants having fruit with reduced endocarp lignification, reduced endocarp lignification and reduced pit hardness, and reduced seediness or a reduced feel of seediness. The development of fruit normally depends on fertilization and the subsequent formation of the seed. Many fruits have large, hard seeds that create an impediment to eating the fruit, with examples ranging from seeded grapes, apples, cherries, and peaches. For some fruits, seedless varieties have been developed which have altered development of the seed after pollination. In examples such as grape and watermelon, the result is often a seed remnant, which is smaller and softer than in seeded varieties, and in some cases this seed remnant is not detected by consumers. The fruit of Prunus plants are stone fruit, which are botanically classified as drupes. A drupe is an indehiscent fruit in which an outer fleshy part (exocarp or skin, and mesocarp or flesh) surrounds a single shell (the pit, stone, or pyrene) of hardened endocarp with a seed (kernel) inside. The endocarp is comprised of lignin to protect the seed or pit. The endocarp develops from the innermost layer of the ovary. The perception of seediness in drupes is caused by the presence of the endocarp-derived pit that encloses the seed. Consequently, seedlessness and/or reduced feeling of ‘seediness’ in fruit is a desirable trait to consumers. The present invention addresses the issue of reducing the hardness of a pit in fruit by mutating NST1 and NST3 genes in Prunus sp. Mutating these genes is believed to reduce or remove the endocarp layer and therefore reduce the feeling of ‘seediness’ or the hardness of the pit.

Accordingly, the present invention provides a Prunus plant or part thereof comprising at least one mutation (e.g., one or more) in at least one endogenous NAC SECONDARY WALL THICKENING PROMOTING FACTOR1 (NST1) gene encoding a NST1 transcription factor polypeptide and at least one mutation in at least one endogenous NAC SECONDARY WALL THICKENING PROMOTING FACTOR3 (NST3) gene encoding a NST3 transcription factor polypeptide, wherein the at least one mutation in the at least one endogenous NST1 gene and/or the at least one mutation in the at least one endogenous NST3 gene is a null mutation or a dominant-negative mutation. In some embodiments, the null mutation is a knockout mutation. In some embodiments, the null mutation results in the production of a truncated polypeptide. In some embodiments, the null mutation results in the production of a non-functional or no NST1 polypeptide and/or a non-functional or no NST3 polypeptide. In some embodiments, the at least one mutation in the at least one endogenous NST1 gene results in a mutation in a dimerization domain (located in Exon 1) and/or transactivating domain (located in Exon 3) of the NST1 transcription factor polypeptide and/or the at least one mutation in the at least one endogenous NST3 gene results in a mutation in a dimerization domain (located in Exon 1) and/or transactivating domain (located in Exon 3) of the NST3 transcription factor polypeptide. In some embodiments, the mutation in the dimerization domain results in a non-functional polypeptide that is unable to dimerize (e.g., unable to dimerize with other NST polypeptides). In some embodiments, the dimerization domain of the NST1 polypeptide comprises a sequence of VPPGFRFHPTEEELLQYYL (SEQ ID NO: 79) and the dimerization domain of the NST3 polypeptide comprises a sequence of VPPGFRFHPTEEELLHYYL (SEQ ID NO:98).

In some embodiments, an endogenous NST1 gene useful with this invention: (a) comprises a sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO: 83; and/or (d) encodes a region having at least 80% identity to the amino acid sequence of any one of SEQ ID NOs: 77-80, optionally wherein the sequence identity of (a), (b), (c) and/or (d) may be at least 85%, or at least 90%, or it may be at least 95%, 95.5%, 96%, 96.5%, 97%, 97.5%, 98%, 98.5%, 99%, or 99.5%, optionally the sequence identity may be 100%; and wherein the endogenous gene encoding an NST3 transcription factor polypeptide: (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO:101; and/or encodes a region having at least 80% identity to the amino acid sequence of SEQ ID NOs: 80 and/or 96-98, optionally wherein the sequence identity of (a), (b), (c) and/or (d) may be at least 85%, or at least 90%, or it may be at least 95%, 95.5%, 96%, 96.5%, 97%, 97.5%, 98%, 98.5%, 99%, or 99.5%, optionally the sequence identity may be 100%. Thus, a plant or plant part of the invention may comprise at least one mutation (e.g., one or more mutations) in an endogenous NST1 gene and/or NST3 gene, wherein the endogenous NST1 gene (a) comprises a sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, optionally wherein the sequence identity of (a), (b), (c) and/or (d) may be at least 85%, or at least 90%, or it may be at least 95%, 95.5%, 96%, 96.5%, 97%, 97.5%, 98%, 98.5%, 99%, or 99.5%, optionally the sequence identity may be 100%; and wherein the endogenous gene encoding an NST3 transcription factor polypeptide: (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO:101; and/or encodes a region having at least 80% identity to the amino acid sequence of SEQ ID NOs: 80 and/or 96-98, optionally wherein the sequence identity of (a), (b), (c) and/or (d) may be at least 85%, or at least 90%, or it may be at least 95%, 95.5%, 96%, 96.5%, 97%, 97.5%, 98%, 98.5%, 99%, or 99.5%, optionally the sequence identity may be 100%.

In some embodiments, an endogenous NST1 gene useful with this invention encodes a NST1 transcription factor polypeptide having the amino acid sequence of SEQ ID NOs: 71 or SEQ ID NO:83. In some embodiments, an endogenous NST3 gene useful with this invention encodes a NST3 transcription factor polypeptide having the amino acid sequence of SEQ ID NO: 90 or SEQ ID NO:101.

A mutation in at least one endogenous NST1 gene and/or at least one endogenous NST3 gene in a plant may be any type of mutation including, but not limited to, a base substitution, a base deletion and/or a base insertion. In some embodiments, a mutation in at least one endogenous NST1 gene and/or a mutation in at least one endogenous NST3 gene may comprise a base substitution to an A, a T, a G, or a C. In some embodiments, a mutation useful with the invention is a non-natural mutation. In some embodiments, a NST1 gene or a NST3 gene may comprise one or more mutations (e.g., 1, 2, 3, 4, 5, 6, 7, 8, 9 or 10 or more).

In some embodiments, a mutation in at least one endogenous NST1 gene and/or at least one endogenous NST3 gene may be at least one mutation located in the first exon of the endogenous NST1 gene encoding a NST1 transcription factor polypeptide and/or at least one mutation located in the first exon of the endogenous NST3 gene encoding a NST3 transcription factor polypeptide. In some embodiments, the at least one mutation is located the first exon of the endogenous NST1 gene and/or is located in the first exon of the endogenous NST3 gene, optionally wherein the first exon of the endogenous NST1 gene comprises a nucleotide sequence having at least 95% sequence identity (e.g., about 95%, 96%, 97%, 98%, 99% or 100% sequence identity) to SEQ ID NO:72 or SEQ ID NO:84 and/or the first exon of the endogenous NST3 gene comprises a nucleotide sequence having at least 95% sequence identity (e.g., about 95%, 96%, 97%, 98%, 99% or 100% sequence identity) to SEQ ID NO:91 or SEQ ID NO:102.

In some embodiments, a mutation in an NST1 gene as described herein comprises a sequence having at least 90% identity to any one of SEQ ID NOs: 125, 126, 129 and 130, and/or a mutation in an NST3 gene as described herein comprises a sequence having at least 90% identity to any one of SEQ ID NOs: 127, 128, 131, and 132. In some embodiments, a mutated NST1 polypeptide as described herein comprises a sequence having at least 90% identity to any one of SEQ ID NOs: 114, 115, 118, and 119, and/or a mutated NST3 polypeptide as described herein comprises a sequence having at least 90% identity to any one of SEQ ID NOs: 116, 117, and 120.

In some embodiments, the endogenous NST1 gene comprises the nucleic acid sequence of SEQ ID NO:69 and/or SEQ ID NO:70 and/or encodes the amino acid sequence of SEQ ID NO:71; and the endogenous NST3 gene comprises the nucleic acid sequence of SEQ ID NO:88 and/or SEQ ID NO:89 and/or encodes the amino acid sequence of SEQ ID NO: 90. In some embodiments, the endogenous NST1 gene comprises the nucleic acid sequence of SEQ ID NO:81 or SEQ ID NO:82 and/or encodes the amino acid sequence of SEQ ID NO:83 and the endogenous NST3 gene comprises the nucleic acid sequence of SEQ ID NO: 99 and/or SEQ ID NO:100 and/or encodes the amino acid sequence of SEQ ID NO: 101.

In some embodiments, the mutation may be a base deletion. In some embodiments, the mutation may be a base insertion. In some embodiments, the base deletion and/or base insertion may be an insertion or deletion of one to about 200 consecutive base pairs (e.g., a deletion or insertion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 175, 180, 185, 190, 195 or 200 consecutive base pairs or any range or value therein), optionally a deletion or insertion of 1 base pair to about 100 consecutive base pairs (e.g., a deletion or insertion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive base pairs or any range or value therein), or a deletion or insertion of 1 base pair to about 45 consecutive base pairs (e.g., a deletion or insertion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 base pairs, or any range or value therein). In some embodiments, a mutation in an endogenous NST1 gene and/or an endogenous NST3 gene may be a deletion of 1 base pair to about 45 consecutive base pairs, optionally a deletion of about 3 to about 30 consecutive base pairs of an endogenous NST1 gene and/or an endogenous NST3 gene (e.g., a deletion of about 3, 4, 5, 6, 7, 8, 9, 10, 11, 12, 13, 14, 15, 16, 17, 18, 19, 20, 21, 22, 23, 24, 25, 26, 27, 28, 29, or 30 base pairs, or any range or value therein). In some embodiments, a mutation in an endogenous NST1 gene and/or an endogenous NST3 gene may be a deletion of at least 3 consecutive base pairs (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or more, or any range or value therein, optionally a deletion of about 3, 4, 5, 6, 7, 8, 9, 10, 11, or 12 consecutive base pairs, or any range or value therein).

In some embodiments, the mutation is in a region of the NST1 gene and/or NST3 gene encoding the dimerization domain having the amino acid sequence VPPGFRFHPTEEELLQYYL (SEQ ID NO:79) or VPPGFRFHPTEEELLHYYL (SEQ ID NO: 98), e.g., a region comprising the amino acid sequence of EEELL (SEQ ID NO:80). In some embodiments, one or more of the amino acid residues (e.g., 1, 2, 3, 4, 5, 6, 7, 8, 9, 10, 11, 12, 13, 14, 15, 16, 17, 18, or 19 amino acid residues, or any range or value therein (e.g., 1 to about 3 residues, 1 to about 4 residues, 1 to about 5 residues, 1 to about 6 residues, 1 to about 8 residues, 1 to about 10 residues, 1 to about 12 residues, 1 to about 14 residues, or 1 to about 19 residues, or any other range or value therein) of the dimerization domain of the NST1 polypeptide and/or NST3 polypeptide are mutated or deleted. In some embodiments, a mutation in an endogenous NST1 gene and/or NST3 gene may be a deletion or insertion that affects the ability of the dimerization domain to dimerize. In some embodiments, a mutation in an endogenous NST1 gene and/or NST3 gene may be an out-of-frame deletion or insertion that affects the ability of the dimerization domain to dimerize. In some embodiments, a mutation in an endogenous NST1 gene and/or NST3 gene may be a deletion that truncates the C-terminus of the NST1 polypeptide and/or NST3 polypeptide, optionally wherein the mutation may be a deletion that deletes the C-terminus comprising at least the dimerization domain (e.g., SEQ ID NO:79 and/or SEQ ID NO:98) of the NST1 polypeptide and/or NST3 polypeptide.

In some embodiments, the mutation may result in an out-of-frame insertion or an in-frame deletion. In some embodiments, the mutation in at least one endogenous NST1 gene is an out-of-frame insertion or an out-of-frame deletion and/or the at least one mutation in an endogenous NST3 gene is an out-of-frame insertion or an out-of-frame deletion. In some embodiments, the at least one mutation in at least one endogenous NST1 gene is an in-frame insertion or an in-frame deletion and/or the at least one mutation in an endogenous NST3 gene is an in-frame insertion or an in-frame deletion.

In some embodiments, a NST1 gene and/or NST3 gene may comprise one mutation or more than one mutation (e.g., insertion or deletion), e.g., 1, 2, 3, 4 or 5 mutations. In some embodiments, the at least one mutation in at least one endogenous NST1 gene results in a deletion or insertion of one or more base pairs located in a region having at least 80% sequence identity (e.g., 80%, 85%, 90%, 95%, or 100% sequence identity) to any one of the nucleotide sequences of SEQ ID NOs: 77-80, and the at least one mutation in at least one endogenous NST3 gene results in a deletion or insertion of one or more base pairs located in a region having at least 80% sequence identity (e.g., 80%, 85%, 90%, 95%, or 100% sequence identity) to any one of the nucleotide sequences of SEQ ID NOs: 80 and/or 96-98. In some embodiments, an endogenous NST1 gene encoding an NST1 transcription factor polypeptide: (a) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO: 71 and/or SEQ ID NO:83; and/or (d) encodes a region having at least 80% identity to the amino acid sequence of any one of SEQ ID NOs: 77-80, and wherein an endogenous NST3 gene encoding an NST3 transcription factor polypeptide: (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO: 90 and/or SEQ ID NO:101; and/or encodes a region having at least 80% identity to the amino acid sequence of SEQ ID NOs: 80 and/or 96-98.

In some embodiments, a mutation (e.g., one or more mutations) in an endogenous NST1 gene and/or an endogenous NST3 gene may result in a dominant allele. In some embodiments, the mutation may be a non-natural mutation.

In some embodiments, a mutation in an endogenous NST1 gene may result in a mutated NST1 gene having at least 90% sequence identity (e.g., 90%, 91%, 92%, 93%, 94%, 95%, 95.5%, 96%, 96.5%, 97%, 97.5%, 98%, 98.5%, 99%, 99.5%, or 100% sequence identity) to a nucleotide sequence described herein and a mutation in an endogenous NST3 gene may result in a mutated NST3 gene having at least 90% sequence identity (e.g., 90%, 91%, 92%, 93%, 94%, 95%, 95.5%, 96%, 96.5%, 97%, 97.5%, 98%, 98.5%, 99%, 99.5%, or 100% sequence identity) to a nucleotide sequence as described herein.

In some embodiments, a mutation in an NST1 gene as described herein comprises a sequence having at least 90% identity to any one of SEQ ID NOs: 125, 126, 129 and 130, and/or a mutation in an NST3 gene as described herein comprises a sequence having at least 90% identity to any one of SEQ ID NOs: 127, 128, 131, and 132. In some embodiments, a mutated NST1 polypeptide as described herein comprises a sequence having at least 90% identity to any one of SEQ ID NOs: 114, 115, 118, and 119, and/or a mutated NST3 polypeptide as described herein comprises a sequence having at least 90% identity to any one of SEQ ID NOs: 116, 117, and 120.

In some embodiments, a plant or part thereof comprising a mutation in a NST1 gene and a NST3 gene as described herein may exhibit a phenotype of reduced endocarp lignification, reduced endocarp lignification and reduced pit hardness, and reduced seediness (reduced pit hardness) or a reduced feel of seediness (a reduced feel of pit hardness or increased softness of the pit), wherein the reduced endocarp lignification, reduced endocarp lignification and reduced pit hardness, and reduced seediness or a reduced feel of seediness is as compared to a control plant or plant part (e.g., an isogenic plant, wild-type unedited plant, or a null segregant) devoid of the NST1 gene mutation and NST3 gene mutation. Reduced endocarp lignification may be characterized by a visible reduction in lignin deposition in the endocarp, e.g., as measured by staining with phloroglucinol-HCl, or the force required to crush a fruit/seed comprising the endocarp. Reduced pit hardness, reduced seediness or a reduced feel of seediness may be measured by the force required to crush a fruit/seed wherein a reduced force is able to crush the seeds when the seeds have reduced seediness or a reduced feel of seediness. Force measurements may be carried out using, e.g., a texture analyzer set in compression mode. In some embodiments, reduced endocarp lignification, reduced endocarp lignification and reduced pit hardness, and reduced seediness or a reduced feel of seediness is as compared to a control plant devoid of the mutation, optionally the comparison is under the same environmental conditions.

In some embodiments, a plant cell comprising an editing system is provided, the editing system comprising: (a) a CRISPR-Cas effector protein; (b) a guide nucleic acid (gRNA, gDNA, crRNA, crDNA) comprising a spacer sequence with complementarity to a region of consecutive nucleotides within an endogenous target gene encoding an NST1 polypeptide; and (c) a guide nucleic acid comprising a spacer sequence with complementarity to a region of consecutive nucleotides within an endogenous target gene encoding an NST3 polypeptide. In some embodiments, an endogenous NST1 gene, to which a spacer sequence of the guide nucleic acid shares complementarity, may encode an NST1 transcription factor polypeptide, wherein the endogenous NST1 gene: (a) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, and wherein the endogenous NST3 gene, to which a spacer sequence of the guide nucleic acid shares complementarity, may encode an NST3 transcription factor polypeptide, wherein the endogenous NST3 gene: (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO: 101; and/or encodes a region having at least 80% identity to the amino acid sequence of SEQ ID NOs: 80 and/or 96-98, optionally wherein the sequence identity of any one of (a), (b), (c) and/or (d) may be at least 85%, or at least 90%, or it may be at least 95%, optionally the sequence identity may be 100%. In some embodiments, a spacer sequence useful with this invention can include, but is not limited to, a nucleotide sequence of any one of SEQ ID NOs: 110-113, or reverse complement thereof, or a combination thereof.

In some embodiments, a Prunus plant cell is provided that comprises at least one mutation in an at least one endogenous NAC SECONDARY WALL THICKENING PROMOTING FACTOR1 (NST1) gene and at least one mutation in at least one endogenous NAC SECONDARY WALL THICKENING PROMOTING FACTOR3 (NST3) gene, wherein the at least one mutation in the NST1 gene and in the NST3 gene is a base deletion that is introduced using an editing system that comprises a nucleic acid binding domain that binds to a target site within the endogenous NST1 gene and a target site within the endogenous NST3 gene, wherein the endogenous NST1 gene: (a) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO: 101; and/or encodes a region having at least 80% identity to the amino acid sequence of SEQ ID NOs: 80 and/or 96-98, optionally wherein the sequence identity of any one of (a), (b), (c) and/or (d) for the NST1 or NST3 gene may be at least 85%, or at least 90%, or it may be at least 95%, optionally the sequence identity may be 100%. In some embodiments, the editing system may further comprise a nuclease, optionally wherein the nuclease is a zinc finger nuclease, transcription activator-like effector nuclease (TALEN), endonuclease (e.g., Fok1) or a CRISPR-Cas effector protein. In some embodiments, the nucleic acid binding domain of the editing system may be from a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein.

In some embodiments, a Prunus plant cell is provided that comprises at least one mutation in an at least one endogenous NST1 gene and at least one mutation in at least one endogenous NST3 gene, wherein the at least one mutation in the NST1 gene and in the NST3 gene is a base deletion that is introduced using an editing system that comprises a nucleic acid binding domain that binds to a target site within the endogenous NST1 gene, wherein the target site within the endogenous NST1 gene is located in a region of the endogenous NST1 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences of SEQ ID NOs: 72-76 and/or 84-87, and wherein the target site within the endogenous NST3 gene is located in a region of the endogenous NST3 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences of SEQ ID NOs: 91-95 and/or 102-105. In some embodiments, the at least one mutation in the endogenous NST1 gene is a null mutation and the at least one mutation in the endogenous NST3 gene is a null mutation. In some embodiments, the Prunus plant cell comprising a null mutation in the NST1 gene and/or a null mutation in the NST3 gene, results in a truncated NST1 polypeptide and/or NST3 polypeptide. In some embodiments, the null mutation results in the production of a non-functional or no NST1 polypeptide and/or non-functional or no NST3 polypeptide. In some embodiments, the at least one mutation in the endogenous NST1 gene is a dominant-negative mutation and the at least one mutation in the endogenous NST3 gene is a dominant-negative mutation. In some embodiments, the at least one mutation in the endogenous NST1 gene is a dominant-negative mutation and the at least one mutation in the endogenous NST3 gene is a null mutation. In some embodiments, the at least one mutation in the endogenous NST1 gene is a null mutation and the at least one mutation in the endogenous NST3 gene is a dominant-negative mutation. In some embodiments, the endogenous NST1 gene or the endogenous NST3 gene has a null mutation. In some embodiments, the endogenous NST1 gene or the endogenous NST3 gene has a dominant-negative mutation. In some embodiments, the Prunus plant cell comprises at least one mutation in an at least one endogenous NST1 gene and at least one mutation in at least one endogenous NST3 gene, wherein the at least one mutation in the endogenous NST1 gene and/or the endogenous NST3 gene is a base deletion. In some embodiments, the at least one mutation is an out-of-frame deletion, out-of-frame insertion, an in-frame deletion or an in-frame insertion. In some embodiments, the at least one mutation is a non-natural mutation, optionally wherein the at least one mutation results in a dominant allele.

In some embodiments, a Prunus plant or part thereof comprising at least one cell having a mutation in an endogenous NST1 gene as described herein comprises a sequence having at least 90% identity to any one of SEQ ID NOs: 125, 126, 129 and 130, and/or encodes a mutated NST1 polypeptide having at least 90% sequence identity to any one of SEQ ID NOs: 114, 115, 118, and 119, optionally wherein the sequence identity may be at least 95%, optionally the sequence identity may be 100%. In some embodiments, a Prunus plant or part thereof comprising at least one cell having a mutation in an endogenous NST3 gene as described herein comprises a sequence having at least 90% identity to any one of SEQ ID NOs: 127, 128, 131, and 132, and/or encodes a mutated NST3 polypeptide having at least 90% sequence identity to any one of SEQ ID NOs: 116, 117, and 120, optionally wherein the sequence identity may be at least 95%, optionally the sequence identity may be 100%.

NST1 genes and/or NST3 genes may be modified to generate a null mutation or dominant-negative mutation by any means known in the art including, but not limited to, traditional plant breeding, molecular breeding, TILLING mutagenesis, RNA interference (RNAi) (gene suppression), and through gene editing using a nuclease linked to a nucleic acid binding domain (e.g., editing system) that binds to a target sequence in the NST1 genes and NST3 genes.

In some embodiments, a nuclease of an editing system useful for generating null mutations or dominant-negative mutations in an NST1 gene and/or an NST3 gene may be a zinc finger nuclease, a transcription activator-like effector nuclease (TALEN), an endonuclease (e.g., Fok1) or a CRISPR-Cas effector protein. In some embodiments, the nucleic acid binding domain of the editing system may be from a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein as described herein and as known in the art.

In some embodiments, expression of an endogenous NST1 gene and an endogenous NST3 gene may be suppressed (reduced or eliminated) in a Prunus plant using RNAi. Use of RNAi to reduce or eliminate expression of an endogenous gene is well-known in the art, see e.g., Kamthan et al. (Frontiers in Plant Science 6: Article 208 (2015) doi.org/10.3389/fpls.2015.00208); Kleter (Pest Manag. Sci. 76:3333-3339 (2020)); Saurabh et al. (Planta 239:543-564 (2014)). RNAi, also known as RNA silencing, inhibitory RNA, and RNA inactivation and includes, but is not limited to, antisense, double stranded (dsRNA), small interfering RNA (siRNA), small hairpin RNA (or short hairpin RNA) (shRNA), microRNA (miRNA), piRNA (PIWI-interacting RNA), qiRNA (QDE-2-interacting RNA), and svRNA (small vault RNA). In some embodiments, an RNAi molecule useful for reducing expression of an NST1 gene or an NST3 gene may be a length of about 18 to 25 consecutive nucleotides, optionally a length of about 20 to 24 consecutive nucleotides (e.g., about 18, 19, 20, 21, 22, 23, 24, or 25 consecutive nucleotides in length or any range therein).

In some embodiments, the present invention provides a method of producing a Prunus plant having at least one NST1 gene with reduced expression (e.g., about 80%-100% reduced; e.g., about 80, 81, 82, 83, 84, 85, 86, 87, 88, 89, 90, 91, 92, 93, 94, 95, 95.5, 96, 96.5, 97, 97.5, 98, 98.5, 99, 99.5, or 100%, or any value or range therein), and at least one NST3 gene with reduced expression (e.g., about 80%-100% reduced; e.g., about 80, 81, 82, 83, 84, 85, 86, 87, 88, 89, 90, 91, 92, 93, 94, 95, 95.5, 96, 96.5, 97, 97.5, 98, 98.5, 99, 99.5, or 100%, or any value or range therein), the method comprising: contacting a population of Prunus plant cells comprising at least one endogenous NST1 gene and at least one endogenous NST3 with an RNAi that targets the NST1 RNA and the NST3 RNA, wherein the RNAi targets a region of the NST1 RNA and the NST3 RNA and results in suppression of expression of the at least one NST1 gene and of the at least one NST3 gene, thereby producing a Prunus plant with reduced expression of at least one NST1 gene and of at least one NST3 gene.

In some embodiments, reduced expression of an NST1 gene and/or an NST3 gene comprises, consists essentially of, or consists of a reduction in expression of about 85% to about 100% or a reduction in expression of about 90% to about 100%. In some embodiments, reduced expression of an NST1 gene and/or an NST3 gene comprises, consists essentially of, or consists of a reduction in expression of about 95, 95.5, 96, 96.5, 97, 97.5, 98, 98.5, 99, 99.5 to about 100% and any range therein. In some embodiments, reduced expression of an NST1 gene and/or an NST3 gene comprises, consists essentially of, or consists of a reduction in expression of the NST1 gene and the NST3 gene of about 100%. In some embodiments, a Prunus plant may be regenerated from Prunus plant cells having at having at least one NST1 gene and at least one NST3 gene having reduced expression as described herein.

A plant or plant part useful with this invention may be a plant in the genus Prunus. Example Prunus plants useful with the invention can include, but are not limited to, P. avium, P. armeniaca, P. cerasus, P. domestica, P. dulcis, P. mume, P. glandulosa, P. persica, or P. salcinia. In some embodiments, the Prunus plant is sweet cherry, sour cherry, peach, apricot, or plum. In some embodiments, a plant may be regenerated from a plant cell or plant part of this invention. In some aspects, a plant cell can be non-propagating plant cell that does not regenerate into a plant. Plants of this invention comprising at least one mutation in a NST1 gene and at least one mutation in a NST3 gene may comprise a reduced endocarp lignification, reduced endocarp lignification and reduced pit (i.e., seed) hardness, and/or reduced seediness and/or a reduced feel of seediness as compared to a plant or part thereof not comprising the same mutations.

The term “reduced endocarp lignification” refers to a decrease in the accumulation of lignin in the pit endocarp, which typically provides a rigid protective layer surrounding the seed. Reduced endocarp lignification may be characterized by a softer pit in fruit of a mutant plant as compared to fruit of a control plant. As used herein, “reduced endocarp lignification” refers to a decrease in pit and/or seed compression, e.g., expressed as peak force (N), of about 15% to about 100% (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any value or range therein) as compared to a control plant (e.g., a plant that is devoid of the mutation or edit). “Reduced pit or seed hardness” refers to a decrease in pit and/or seed compression, e.g., expressed as peak force (N), of about 15% to about 100% (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any value or range therein) as compared to a control plant (e.g., a plant that is devoid of the mutation or edit).

The term “reduced seediness” refers to an improvement in the number or perception of the number of seeds of a fruit by about 15% to about 100% (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any value or range therein) as compared to a control plant (e.g., a plant that is devoid of the mutation or edit).

As used herein, “reduced feel of seediness” refers to non-taste-related aspects of the pleasantness experienced by a person while chewing or swallowing a fruit. As used herein, “reduced feel of seediness” refers to a decrease in the feel of seediness of about 15% to about 100% (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any value or range therein) as compared to a control plant (e.g., a plant that is devoid of the mutation or edit).

Also provided herein is a method of providing a plurality of plants having reduced endocarp lignification, reduced endocarp lignification and reduced pit hardness, reduced seediness and/or a reduced feel of seediness, the method comprising planting two or more plants of the invention (e.g., 2, 3, 4, 5, 6, 7, 8, 9, 10, 20, 30, 40, 50, 100, 200, 300, 400, 500, 1000, 2000, 3000, 400, 5000, or 10,000 or more plants of the invention) (e.g., comprising a mutation in a NST1 gene and a mutation in a NST3 gene and having reduced endocarp lignification, reduced endocarp lignification and reduced pit hardness, reduced seediness and/or a reduced feel of seediness) in a growing area, thereby providing a plurality of plants having reduced endocarp lignification, reduced endocarp lignification and reduced pit hardness, reduced seediness and/or a reduced feel of seediness as compared to a plurality of control plants not comprising the at least one mutation (e.g., as compared to an isogenic wild-type plant not comprising the mutation). A growing area can be any area in which a plurality of plants can be planted together, including, but not limited to, a field (e.g., a cultivated field, an agricultural field), a growth chamber, a greenhouse, a recreational area, a lawn, and/or a roadside, and the like.

In some embodiments, a method of producing/breeding a transgene-free genome-edited (e.g., base-edited) Prunus plant is provided, the method comprising: crossing a plant of the present invention (e.g., a Prunus plant comprising a mutation or modification in an endogenous NST1 gene and a mutation in an endogenous NST3 gene as described herein (and having reduced endocarp lignification, reduced endocarp lignification and reduced pit hardness, reduced seediness and/or a reduced feel of seediness) with a transgene-free plant, thereby introducing the mutation or modification (e.g., one or more mutations or modifications) into the plant that is transgene-free (e.g., into progeny plants); and selecting a progeny plant that comprises the mutation or modification and is transgene-free, thereby producing a transgene-free genome-edited (e.g., base-edited) plan. In some embodiments, the mutation may be a non-natural mutation.

In some embodiments, the present invention provides a method of creating a mutation in an endogenous NST1 gene and an endogenous NST3 gene in a Prunus plant, comprising: (a) targeting a gene editing system to a portion of the endogenous NST1 gene and the endogenous NST3 gene, wherein the portion of the endogenous NST1 gene that is targeted comprises a sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 72-76 and/or 84-87, and wherein the portion of the endogenous NST3 gene that is targeted comprises a sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 91-95 and/or 102-105; and (b) selecting a Prunus plant that comprises a modification located in the portion of the endogenous NST1 gene that comprises a nucleotide sequence having at least 80% sequence identity to any one of the nucleotide sequences of SEQ ID NOs: 72-76 and/or 84-87 and a modification located in the portion of the endogenous NST3 gene that comprises a nucleotide sequence having at least 80% sequence identity to any one of the nucleotide sequences of SEQ ID NOs: 91-95 and/or 102-105. In some embodiments, the mutation that is created results in a nucleic acid having at least 90% sequence identity to mutant NST1 gene or mutant NST3 gene.

In some embodiments, a method of generating variation in region of an endogenous NST1 gene and an endogenous NST3 gene, comprising: into a Prunus plant cell (a) an editing system that is targeted to a region of a NST1 gene and (b) an editing system that is targeted to a region of an NST3 gene, and contacting the region of the NST1 gene with the editing system of (a), thereby introducing a mutation into the NST1 gene and generating variation in the NST1 gene of the Prunus plant cell, and contacting the region of the NST3 gene with the editing system of (b), thereby introducing a mutation into the NST3 gene and generating variation in the NST3 gene of the Prunus plant cell. In some embodiments, the endogenous NST1 gene encodes an amino acid sequence having at least 95% sequence identity to the amino acid sequence of SEQ ID NO:71 and/or the endogenous NST3 gene encodes an amino acid sequence having at least 95% sequence identity to the amino acid sequence of SEQ ID NO: 90, optionally wherein the sequence identity may be at least 95%, or at least 96%, or it may be at least 97%, 98% or 99%, optionally the sequence identity may be 100%. In some embodiments, the endogenous NST1 gene encodes the amino acid sequence of SEQ ID NO: 83 and/or the endogenous NST3 gene encodes the amino acid sequence of SEQ ID NO: 101. In some embodiments, the region of the endogenous NST1 gene that is targeted comprises a sequence having at least 80% sequence identity to any one of the nucleotide sequences of SEQ ID NOs: 72-76 and/or 84-87, and the region of the endogenous NST3 gene that is targeted comprises a sequence having at least 80% sequence identity to any one of the nucleotide sequences of SEQ ID NOs: 91-95 and/or 102-105. In some embodiments, contacting the region of the endogenous NST1 gene and the region of the endogenous NST3 gene in the Prunus plant cell with the editing system produces a Prunus plant cell comprising in its genome an edited NST1 gene and an edited NST3 gene. In some embodiments, the method may further comprise (a) regenerating a Prunus plant from the Prunus plant cell; (b) selfing the plant to produce progeny plants (E1); (c) assaying the progeny plants of (b) for reduced endocarp lignification, reduced endocarp lignification and reduced pit hardness, reduced seediness and/or a reduced feel of seediness; and (d) selecting the progeny plants exhibiting reduced endocarp lignification, reduced endocarp lignification and reduced pit hardness, reduced seediness and/or a reduced feel of seediness as compared to a control plant. In some embodiments, the method may further comprise (e) selfing the selected progeny plants of (d) to produce progeny plants (E2); (f) assaying the progeny plants of (e) for reduced endocarp lignification, reduced endocarp lignification and reduced pit hardness, reduced seediness and/or a reduced feel of seediness; and (g) selecting the progeny plants exhibiting re reduced endocarp lignification, reduced endocarp lignification and reduced pit hardness, reduced seediness and/or a reduced feel of seediness as compared to a control plant, optionally repeating (e) through (g) one or more additional times.

In some embodiments, a method of detecting a mutant NST1 gene (a mutation in an endogenous NST1 gene) and a mutant NST3 gene (a mutation in an endogenous NST3 gene) in a plant or plant part (e.g., plant cell) is provided, the method comprising detecting in the genome of the plant a NST1 gene having at least one mutation in a region having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 72-76 and/or 84-87 and detecting in the genome of the plant a NST3 gene having at least one mutation in a region having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 91-95 and/or 102-105, optionally wherein the mutation is a deletion and/or an insertion of at least one nucleotide (e.g., 1, 2, 3, 4, 5, 6, 7, 8, 9, 10 consecutive nucleotides to 50, 60, 70, 80, 90, 100 consecutive nucleotides to about 200 consecutive nucleotides). n some embodiments, the mutant NST1 gene and/or mutant NST3 gene that is detected comprises a nucleic acid sequence having at least 90% sequence identity to a mutant NST1 gene and/or mutant NST3 gene described herein.

In some embodiments, a mutant NST1 gene that is generated or detected comprises a sequence having at least 90% identity to any one of SEQ ID NOs: 125, 126, 129 and 130, and/or a encodes a mutated NST1 polypeptide comprising a sequence having at least 90% identity to any one of SEQ ID NOs: 114, 115, 118, and 119. In some embodiments, a mutant NST3 gene that is generated or detected comprises a sequence having at least 90% identity to any one of SEQ ID NOs: 127, 128, 131, and 132, encodes a mutated NST3 polypeptide comprising a sequence having at least 90% identity to any one of SEQ ID NOs: 116, 117, and 120.

In some embodiments, a method for editing a specific site in the genome of a Prunus plant cell is provided, the method comprising cleaving, in a site-specific manner, a target site within an endogenous NAC SECONDARY WALL THICKENING PROMOTING FACTOR1 (NST1) gene in the Prunus plant cell and a target site within an endogenous NAC SECONDARY WALL THICKENING PROMOTING FACTOR3 (NST3) gene in the Prunus plant cell, wherein the endogenous NST1 gene: (a) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, optionally wherein the sequence identity of (a), (b), (c) and/or (d) may be at least 85%, or at least 90%, or it may be at least 95%, 95.5%, 96%, 96.5%, 97%, 97.5%, 98%, 98.5%, 99%, or 99.5%, optionally the sequence identity may be 100%; and wherein the endogenous NST3 gene (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO:101; and/or encodes a region having at least 80% identity to the amino acid sequence of SEQ ID NOs: 80 and/or 96-98, optionally wherein the sequence identity of (a), (b), (c) and/or (d) may be at least 85%, or at least 90%, or it may be at least 95%, 95.5%, 96%, 96.5%, 97%, 97.5%, 98%, 98.5%, 99%, or 99.5%, optionally the sequence identity may be 100%, thereby generating an edit in the endogenous NST1 and in the endogenous NST3 gene of the Prunus plant cell, optionally wherein the sequence identity of any one of (a), (b), (c) and/or (d) may be at least 85%, or at least 90%, or it may be at least 95%, optionally the sequence identity may be 100. In some embodiments, the edit in the endogenous NST1 gene is in Exon 1 of the endogenous NST1 gene and the edit in the endogenous NST3 gene is in Exon 1 of the endogenous NST3 gene. In some embodiments, the edit in the endogenous NST1 gene and/or the endogenous NST3 gene results in a null allele. In some embodiments, the edit in the endogenous NST1 gene and/or the endogenous NST3 gene results in a dominant-negative allele. In some embodiments, the edit results in a mutation, optionally a non-natural mutation, in the endogenous NST1 gene and/or endogenous NST3 gene, optionally wherein mutation results in a truncated polypeptide. In some embodiments, the plant comprising the edit in its endogenous NST1 gene and endogenous NST3 gene exhibits reduced endocarp lignification, reduced endocarp lignification and reduced pit hardness, reduced seediness and/or a reduced feel of seediness as compared to a control plant that is devoid of the edit. In some embodiments, the edit may be located in a region of the NST1 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally at least 90% or 95%, optionally 100%) to any one of the nucleotide sequences of SEQ ID NOs: 72-76 and/or 84-87, and the edit may be located in a region of the NST3 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally at least 90% or 95%, optionally 100%) to any one of the nucleotide sequences of SEQ ID NOs: 91-95 and/or 102-105. In some embodiments, an edit in an endogenous NST1 gene and edit in an endogenous NST3 gene may result in a mutated NST1 gene and mutated NST3 gene having at least 90% sequence identity (e.g., at least 90, 91, 92, 93, 94, 95, 96, 97, 98, 99 or 100%, optionally the sequence identity may be at least 95%, optionally the sequence identity may be 100%) to mutant NST1 gene and mutant NST3 gene as described herein, and/or encode a modified NST1 polypeptide and encode a modified NST3 polypeptide comprising an amino acid sequence having at least 90% sequence identity to a modified NST1 polypeptide and modified NST3 polypeptide as described herein.

In some embodiments, a Prunus plant may be regenerated from a plant cell comprising an edit in an endogenous NST1 gene and an edit in an endogenous NST3 gene to produce a Prunus plant comprising the edit in its endogenous NST1 gene and endogenous NST3 gene. In some embodiments, a Prunus plant is not regenerated from a plant cell. In some embodiments, a Prunus plant comprising an edit in its endogenous NST1 gene and endogenous NST3 gene reduced endocarp lignification, reduced endocarp lignification and reduced pit hardness, reduced seediness and/or a reduced feel of seediness as compared to a control Prunus plant that is devoid of the edit.

In some embodiments, a method for making a Prunus plant is provided, the method comprising: (a) contacting a population of Prunus plant cells that comprise an endogenous gene encoding a NAC SECONDARY WALL THICKENING PROMOTING FACTOR1 (NST1) polypeptide and an endogenous gene encoding an endogenous NAC SECONDARY WALL THICKENING PROMOTING FACTOR3 (NST3) polypeptide with a nuclease targeted to the endogenous gene encoding the NST1 polypeptide and a nuclease targeted to the endogenous gene encoding the NST1 polypeptide, wherein each nuclease is linked to a nucleic acid binding domain that binds to a target site within the endogenous gene, wherein the endogenous gene encoding an NST1 polypeptide: (i) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (ii) comprises a region having at least 80% sequence identity to any one of the nucleotide sequences of SEQ ID NOs: 72-76 and/or 84-87; (iii) encodes a NST1 transcription factor polypeptide having at least 80% sequence identity to the amino acid sequence of SEQ ID NO:71 and/or SEQ ID NO:83; and/or (iv) encodes a region having at least 80% identity to the amino acid sequence of any one of SEQ ID NOs: 77-80, optionally wherein the sequence identity of (i), (ii), (iii) and/or (iv) may be at least 85%, or at least 90%, or it may be at least 95%, optionally the sequence identity may be 100%, and wherein the endogenous gene encoding an NST3 polypeptide: (i) comprises a nucleotide sequence having at least 80% sequence identity to any one of SEQ ID NOs: 88, 89, 99, or 100; (ii) comprises a region having at least 80% sequence identity to a nucleotide sequence of any one of SEQ ID NOs: 91-95 and/or 102-105; (iii) encodes a NST3 transcription factor polypeptide having at least 80% sequence identity to the amino acid sequence of SEQ ID NO:90 and/or SEQ ID NO:101; and/or (iv) encodes a region having at least 80% identity to the amino acid sequence of SEQ ID NOs: 80 and/or 96-98, optionally wherein the sequence identity of (i), (ii), (iii) and/or (iv) may be at least 85%, or at least 90%, or it may be at least 95%, optionally the sequence identity may be 100%, (b) selecting a Prunus plant cell from the population comprising a mutation in the endogenous gene encoding a NST1 polypeptide and a mutation in the endogenous gene encoding a NST3 polypeptide, wherein the mutation is a deletion; and (c) growing the selected Prunus plant cell into a Prunus plant comprising the mutation in the endogenous gene encoding a NST1 polypeptide and the mutation in the endogenous gene encoding a NST3 polypeptide. In some embodiments, the mutation in an endogenous NST1 gene and mutation in an endogenous NST3 gene may result in a mutated NST1 gene and mutated NST3 gene having at least 90% sequence identity (e.g., at least 90, 91, 92, 93, 94, 95, 96, 97, 98, 99 or 100%, optionally the sequence identity may be at least 95%, optionally the sequence identity may be 100%) to a mutant NST1 gene and mutant NST3 gene as described herein, and/or encode a modified NST1 polypeptide and encode a modified NST3 polypeptide comprising an amino acid sequence having at least 90% sequence identity to a modified NST1 polypeptide and modified NST3 polypeptide as described herein.

In some embodiments, a method for reducing endocarp lignification, reducing endocarp lignification and pit hardness, seediness and/or a reducing the feel of seediness of a Prunus plant is provided, the method comprising (a) contacting a Prunus plant cell comprising an endogenous gene encoding a NAC SECONDARY WALL THICKENING PROMOTING FACTOR1 (NST1) polypeptide and comprising an endogenous gene encoding a NAC SECONDARY WALL THICKENING PROMOTING FACTOR3 (NST3) with a nuclease targeted to the endogenous gene encoding the NST1 polypeptide and a nuclease targeted to the endogenous gene encoding the NST3 polypeptide, wherein each nuclease is linked to a nucleic acid binding domain that binds to a target site within the endogenous gene, wherein the endogenous NST1 gene: (i) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (ii) comprises a region having at least 80% sequence identity to any one of the nucleotide sequences of SEQ ID NOs: 72-76 and/or 84-87; (iii) encodes a NST1 transcription factor polypeptide having at least 80% sequence identity to the amino acid sequence of SEQ ID NO:71 and/or SEQ ID NO:83; and/or (iv) encodes a region having at least 80% identity to the amino acid sequence of any one of SEQ ID NOs: 77-80, optionally wherein the sequence identity of (i), (ii), (iii) and/or (iv) may be at least 85%, or at least 90%, or it may be at least 95%, optionally the sequence identity may be 100%, and wherein the endogenous gene encoding an NST3 polypeptide: (i) comprises a nucleotide sequence having at least 80% sequence identity to any one of SEQ ID NOs: 88, 89, 99, or 100; (ii) comprises a region having at least 80% sequence identity to a nucleotide sequence of any one of SEQ ID NOs: 91-95 and/or 102-105; (iii) encodes a NST3 transcription factor polypeptide having at least 80% sequence identity to the amino acid sequence of SEQ ID NO:90 and/or SEQ ID NO:101; and/or (iv) encodes a region having at least 80% identity to the amino acid sequence of SEQ ID NOs: 80 and/or 96-98, optionally wherein the sequence identity of (i), (ii), (iii) and/or (iv) may be at least 85%, or at least 90%, or it may be at least 95%, optionally the sequence identity may be 100%; and (b) growing the Prunus plant cell into a Prunus plant, thereby reducing endocarp lignification, reducing endocarp lignification and reducing pit hardness, reducing seediness and/or a reducing the feel of seediness of the Prunus plant. In some embodiments, the regenerated plant comprises a mutated NST1 gene and a mutated NST3 gene having at least 90% sequence identity (e.g., at least 90, 91, 92, 93, 94, 95, 96, 97, 98, 99 or 100%, optionally the sequence identity may be at least 95%, optionally the sequence identity may be 100%) to a mutant nucleic acid sequence as described herein and/or encodes an amino acid sequence having at least 90% sequence identity to a mutant amino acid sequence as described herein.

In some embodiments, a method is provided for producing a Prunus plant or part thereof comprising at least one cell (e.g., one or more cells) having a mutation in an endogenous NST1 gene and a mutation in an endogenous NST3 gene, the method comprising contacting a target site within the endogenous NST1 gene and a target site within the endogenous NST3 gene in the Prunus plant or part thereof with a nuclease comprising a cleavage domain and a nucleic acid binding domain, wherein the nucleic acid binding domain of the nuclease binds to a target site within the endogenous NST1 gene and a target site within the endogenous NST3 gene, and/or contacting a target site within the endogenous NST1 gene and a target site within the endogenous NST3 gene in the Prunus plant or part thereof with a first nuclease and a second nuclease each of which comprise a cleavage domain and a nucleic acid binding domain, wherein the nucleic acid binding domain of the first nuclease binds to a target site within the endogenous NST1 gene and the nucleic acid binding domain of the second nuclease binds to a target site within the endogenous NST3 gene, wherein the endogenous NST1 gene: (a) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO: 101; and/or (d) encodes a region having at least 80% identity to the amino acid sequence of SEQ ID NOs: 80 and/or 96-98, optionally wherein the sequence identity of any one of (a), (b), (c) and/or (d) for the NST1 or NST3 gene may be at least 85%, or at least 90%, or it may be at least 95%, optionally the sequence identity may be 100%, thereby producing a Prunus plant or part thereof comprising at least one cell having a mutation in the endogenous NST1 gene and in the endogenous NST3 gene. In some embodiments, the mutation in the endogenous NST1 gene may be located in the 5′ region of the endogenous NST1 gene and the mutation in the endogenous NST3 gene may be located in the 5′ region of the endogenous NST3 gene. In some embodiments, the mutation in the endogenous NST1 gene may be in Exon 1 of the endogenous NST1 gene and the mutation in the endogenous NST3 gene may be in Exon 1 of the endogenous NST3 gene. In some embodiments, a mutation may result in a deletion, optionally wherein the deletion results in a truncated NST1 polypeptide and/or truncated NST3 polypeptide. In some embodiments, the Prunus plant that is produced comprises a mutated NST1 gene and NST3 gene having at least 90% sequence identity (e.g., at least 90, 91, 92, 93, 94, 95, 96, 97, 98, 99 or 100%, optionally the sequence identity may be at least 95%, optionally the sequence identity may be 100%) to a mutant nucleic acid describe herein and/or encodes a mutant amino acid sequence as described herein.

In some embodiments, a method is provided for producing a Prunus plant or part thereof comprising a mutation in an endogenous NST1 gene and an endogenous NST3 gene and having reduced endocarp lignification, reduced endocarp lignification and reduced pit hardness, reduced seediness and/or a reduced feel of seediness, the method comprising contacting a target site within the endogenous NST1 gene and a target site within the endogenous NST3 gene in the Prunus plant or part thereof with a nuclease comprising a cleavage domain and a nucleic acid binding domain, wherein the nucleic acid binding domain of the nuclease binds to a target site within the endogenous NST1 gene and a target site within the endogenous NST3 gene, and/or contacting a target site within the endogenous NST1 gene and a target site within the endogenous NST3 gene in the Prunus plant or part thereof with a first nuclease and a second nuclease each of which comprise a cleavage domain and a nucleic acid binding domain, wherein the nucleic acid binding domain of the first nuclease binds to a target site within the endogenous NST1 gene and the nucleic acid binding domain of the second nuclease binds to a target site within the endogenous NST3 gene, wherein the endogenous NST1 gene: (a) comprises a nucleic acid sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to the nucleotide sequence of any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO: 101; and/or (d) encodes a region having at least 80% identity to the amino acid sequence of SEQ ID NOs: 80 and/or 96-98, optionally wherein the sequence identity of any one of (a), (b), (c) and/or (d) may be at least 85%, or at least 90%, or it may be at least 95%, optionally the sequence identity may be 100%, thereby producing a Prunus plant or part thereof having a mutated endogenous NST1 gene and a mutated endogenous NST3 gene and exhibiting reduced endocarp lignification, reduced endocarp lignification and reduced pit hardness, reduced seediness and/or a reduced feel of seediness as compared to a control Prunus plant.

In some embodiments, a mutant NST1 nucleic acid as described herein comprises a sequence having at least 90% identity to any one of SEQ ID NOs: 125, 126, 129 and 130 and/or a encodes a mutated NST1 polypeptide comprising a sequence having at least 90% identity to any one of SEQ ID NOs: 114, 115, 118, and 119, optionally wherein the sequence identity may be at least 95%, optionally the sequence identity may be 100%. In some embodiments, a mutant NST3 nucleic acid as described herein comprises a sequence having at least 90% identity to any one of SEQ ID NOs: 127, 128, 131, and 132 and/or a encodes a mutated NST3 polypeptide comprising a sequence having at least 90% identity to any one of SEQ ID NOs: 116, 117, and 120, optionally wherein the sequence identity may be at least 95%, optionally the sequence identity may be 100%.

In some embodiments, the nuclease may be a zinc finger nuclease, transcription activator-like effector nucleases (TALEN), endonuclease (e.g., Fok1) or a CRISPR-Cas effector protein, wherein the nuclease cleaves the endogenous NST1 gene and the endogenous NST3 gene and a mutation is introduced into the endogenous NST1 gene and into the endogenous NST3 gene; or wherein the first nuclease cleaves the endogenous NST1 gene and a mutation is introduced into the endogenous NST1 gene and the second nuclease cleaves the endogenous NST3 gene and a mutation is introduced into the endogenous NST3 gene. In some embodiments, the target site is in a region of the NST1 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences of SEQ ID NOs: 72-76 and/or 84-87, and the target site is in a region of the NST3 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences of SEQ ID NOs: 91-95 and/or 102-105.

In some embodiments, the mutation that is introduced into the endogenous NST1 gene may be located in Exon 1 of the endogenous NST1 gene and the mutation that is introduced into the endogenous NST3 gene may be located in Exon 1 of the endogenous NST3 gene. In some embodiments, the mutation may be a base deletion optionally a base deletion of at least one base, optionally a non-natural mutation. In some embodiments, a mutation may be a base deletion, wherein the base deletion may be a deletion of about one base pair to about 3 base pairs, a deletion of about 5 base pairs, a deletion of about 10 base pairs, a deletion of about 12 base pairs, a deletion of about 15 base pairs, a deletion of about 20 base pairs, a deletion of about 25 base pairs, a deletion of about 30 base pairs, a deletion of about 35 base pairs, a deletion of about 40 base pairs, a deletion of about 45 base pairs, a deletion of about 50 base pairs, optionally the base deletion may be a deletion of between 5 and 50 base pairs. In some embodiments, a NST1 gene and/or a NST3 gene may comprise more than one mutation, e.g., 1, 2, 3, 4 or 5 or more mutations, which mutations may be deletions. In some embodiments, a mutation in an endogenous NST1 gene and/or a NST3 gene may result in a dominant allele.

In some embodiments, a plant or part thereof that is produced by the methods of this invention comprises a mutated endogenous NST1 gene and a mutated endogenous NST3 gene as described herein and exhibits reduced endocarp lignification, reduced endocarp lignification and reduced pit hardness, reduced seediness and/or a reduced feel of seediness as compared to a control plant that is devoid of the mutation in the endogenous NST1 gene and devoid of the mutation in the endogenous NST3 gene.

In some embodiments, a nuclease contacting a Prunus plant cell, a population of Prunus plant cells and/or a target site cleaves an endogenous NST1 gene and an endogenous NST3 gene, thereby introducing a mutation into Exon 1 of the endogenous NST1 gene and into Exon 1 of the endogenous NST3 gene. A nuclease useful with the invention may be any nuclease that can be utilized to edit/modify a target nucleic acid. Such nucleases include, but are not limited to, a zinc finger nuclease, transcription activator-like effector nucleases (TALEN), endonuclease (e.g., Fok1) and/or a CRISPR-Cas effector protein. Likewise, any nucleic acid binding domain (e.g., DNA binding domain) useful with the nuclease of the invention may be any nucleic acid binding domain that can be utilized to edit/modify a target nucleic acid. Such a nucleic acid binding domain includes, but is not limited to, a zinc finger, transcription activator-like DNA binding domain (TAL), an argonaute and/or a CRISPR-Cas effector DNA binding domain.

In some embodiments, a method is provided for modifying an endogenous NST1 gene and an endogenous NST3 gene in a Prunus plant or part thereof for reducing endocarp lignification, reduced endocarp lignification and reduced pit hardness, reduced seediness and/or a reduced feel of seediness in the Prunus plant or part thereof, the method comprising modifying a target site within the endogenous NST1 gene and a target site within the endogenous NST3 gene in the Prunus plant or a part thereof, wherein the endogenous NST1 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO: 83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST1 transcription factor polypeptide having at least 80% sequence identity to the amino acid sequence of SEQ ID NO:90 and/or SEQ ID NO:101; and/or (d) comprises a region having at least 80% sequence identity to the amino acid sequence of any one of SEQ ID NOs: 80 and/or 96-98, optionally wherein the sequence identity of any one of (a), (b), (c) and/or (d) may be at least 85%, or at least 90%, or it may be at least 95%, optionally the sequence identity may be 100%, thereby modifying the endogenous NST1 gene and modifying the endogenous NST3 gene and reducing endocarp lignification, reduced endocarp lignification and reduced pit hardness, reduced seediness and/or a reduced feel of seediness in the Prunus plant or part thereof. In some embodiments, the target site is a region of the NST1 gene having at least 80% sequence identity to a nucleotide sequence of any one SEQ ID NOs: 72-76 and/or 84-87 and/or the target site is a region of the NST3 gene having at least 80% sequence identity to a nucleotide sequence of any one SEQ ID NOs: 91-95 and/or 102-105.

In some embodiments, a method of editing an endogenous NST1 gene and endogenous NST3 gene in a Prunus plant or plant part is provided, the method comprising contacting a target site within the NST1 gene and NST3 gene in the Prunus plant or plant part with a base editing system comprising a cytosine deaminase and a nucleic acid binding domain that binds to a target site within the NST1 gene and NST3 gene, wherein the endogenous NST1 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO: 71 and/or SEQ ID NO:83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO: 101; and/or encodes a region having at least 80% identity to the amino acid sequence of SEQ ID NOs: 80 and/or 96-98, optionally wherein the sequence identity of any one of (a), (b), (c) and/or (d) may be at least 85%, or at least 90%, or it may be at least 95%, optionally the sequence identity may be 100%, thereby producing the plant or part thereof comprising an endogenous NST1 gene having a mutation and an endogenous NST3 gene having a mutation. In some embodiments, a plant comprising the endogenous NST1 gene and an endogenous NST3 gene that comprises a mutation as described herein exhibits reduced endocarp lignification, reduced endocarp lignification and reduced pit hardness, reduced seediness and/or a reduced feel of seediness in the Prunus plant or part thereof as compared to a plant devoid of the mutation.

In some embodiments, a method of editing an endogenous NST1 gene and endogenous NST3 gene in a Prunus plant or plant part is provided, the method comprising contacting a target site within the NST1 gene and NST3 gene in the Prunus plant or plant part with a base editing system comprising an adenosine deaminase and a nucleic acid binding domain that binds to a target site within the NST1 gene and NST3 gene, wherein the endogenous NST1 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO: 71 and/or SEQ ID NO:83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO: 101; and/or encodes a region having at least 80% identity to the amino acid sequence of SEQ ID NOs: 80 and/or 96-98, optionally wherein the sequence identity of any one of (a), (b), (c) and/or (d) may be at least 85%, or at least 90%, or it may be at least 95%, optionally the sequence identity may be 100%, thereby producing the plant or part thereof comprising an endogenous NST1 gene having a mutation and an endogenous NST3 gene having a mutation. In some embodiments, a plant comprising the endogenous NST1 gene and an endogenous NST3 gene that comprises a mutation as described herein exhibits reduced endocarp lignification, reduced endocarp lignification and reduced pit hardness, reduced seediness and/or a reduced feel of seediness in the Prunus plant or part thereof as compared to a plant devoid of the mutation.

In some embodiments, the present invention provides a method of producing a plant comprising a mutation in an endogenous NST1 gene and an endogenous NST3 gene and at least one polynucleotide of interest, the method comprising crossing a plant of the invention comprising at least one mutation in an endogenous NST1 gene and an endogenous NST3 gene (a first plant) with a second plant that comprises the at least one polynucleotide of interest to produce progeny plants; and selecting progeny plants comprising at least one mutation in the NST1 gene and an endogenous NST3 gene and the at least one polynucleotide of interest, thereby producing the plant comprising a mutation in an endogenous NST1 gene and an endogenous NST3 gene and at least one polynucleotide of interest.

Further provided is a method of producing a plant comprising a mutation in an endogenous NST1 gene and an endogenous NST3 gene and at least one polynucleotide of interest, the method comprising introducing at least one polynucleotide of interest into a plant of the present invention comprising at least one mutation in a NST1 gene and an endogenous NST3 gene, thereby producing a plant comprising at least one mutation in a NST1 gene and in an endogenous NST3 gene and at least one polynucleotide of interest. In some embodiments, the polynucleotide of interest is a polynucleotide that confers herbicide tolerance, insect resistance, disease resistance, increased yield, increased nutrient use efficiency or abiotic stress resistance.

Additionally provided is a method of producing a plant comprising a mutation in an endogenous NST1 gene and a mutation in an endogenous NST3 gene exhibiting a phenotype of reduced endocarp lignification, reduced endocarp lignification and reduced pit hardness, reduced seediness and/or a reduced feel of seediness, comprising crossing a first plant, which is the plant of the present invention (e.g., comprising at least one mutation in an endogenous NST1 gene and at least one mutation in an endogenous NST3 gene), with a second plant that exhibits a phenotype of reduced endocarp lignification, reduced endocarp lignification and reduced pit hardness, reduced seediness and/or a reduced feel of seediness; and selecting progeny plants comprising the mutation in the NST1 gene and the mutation in the NST3 gene and a phenotype of reduced endocarp lignification, reduced endocarp lignification and reduced pit hardness, reduced seediness and/or a reduced feel of seediness, thereby producing the plant comprising a mutation in an endogenous NST1 gene and a mutation in an endogenous NST3 gene and exhibiting a phenotype of reduced endocarp lignification, reduced endocarp lignification and reduced pit hardness, reduced seediness and/or a reduced feel of seediness as compared to a control plant.

Further provided is a method of controlling weeds in a container (e.g., pot, or seed tray and the like), a growth chamber, a greenhouse, a field, a recreational area, a lawn, or on a roadside, the method comprising applying an herbicide to one or more (a plurality) plants of the present invention (e.g., comprising at least one mutation in an endogenous NST1 gene and at least one mutation in an endogenous NST3 gene) growing in a container, a growth chamber, a greenhouse, a field, a recreational area, a lawn, or on a roadside, thereby controlling the weeds in the container, the growth chamber, the greenhouse, the field, the recreational area, the lawn, or on the roadside in which the one or more plants are growing.

In some embodiments, a method of reducing insect predation on a plant is provided, the method comprising applying an insecticide to one or more plants of the invention (e.g., comprising at least one mutation in an endogenous NST1 gene and at least one mutation in an endogenous NST3 gene), thereby reducing insect predation on the one or more plants.

In some embodiments, a method of reducing fungal disease on a plant is provided, the method comprising applying a fungicide to one or more plants of the invention (e.g., comprising at least one mutation in an endogenous NST1 gene and at least one mutation in an endogenous NST3 gene), thereby reducing fungal disease on the one or more plants, optionally wherein the one or more plants are growing in a container, a growth chamber, a greenhouse, a field, a recreational area, a lawn, or on a roadside.

In some embodiments, a method of reducing bacterial disease on a plant is provided, the method comprising applying a bactericide to one or more plants of the invention (e.g., comprising at least one mutation in an endogenous NST1 gene and at least one mutation in an endogenous NST3 gene), thereby reducing bacterial disease on the one or more plants, optionally wherein the one or more plants are growing in a container, a growth chamber, a greenhouse, a field, a recreational area, a lawn, or on a roadside.

A polynucleotide of interest may be any polynucleotide that can confer a desirable phenotype or otherwise modify the phenotype or genotype of a plant. In some embodiments, a polynucleotide of interest may include, but is not limited to, a polynucleotide that confers herbicide tolerance, insect resistance, nematode resistance, disease resistance, increased yield, increased nutrient use efficiency and/or abiotic stress resistance.

Thus, plants or plant cultivars which are to be treated with preference in accordance with the invention include all plants which, through genetic modification, received genetic material which imparts particular advantageous useful properties (“traits”) to these plants. Examples of such properties are better plant growth, vigor, stress tolerance, standability, lodging resistance, nutrient uptake, plant nutrition, and/or yield, in particular improved growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products.

A NST1 gene useful with this invention includes any endogenous NST1 gene in which a mutation as described herein can confer reduced endocarp lignification, reduced endocarp lignification and reduced pit hardness, reduced seediness and/or a reduced feel of seediness in a plant or part thereof comprising the mutation. In some embodiments, a NST1 gene (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, optionally wherein the sequence identity of (a), (b), (c) and/or (d) may be at least 85%, or at least 90%, or it may be at least 95%, optionally the sequence identity may be 100%.

A NST3 gene useful with this invention includes any endogenous NST3 gene in which a mutation as described herein can confer reduced endocarp lignification, reduced endocarp lignification and reduced pit hardness, reduced seediness and/or a reduced feel of seediness in a plant or part thereof comprising the mutation. In some embodiments, a NST3 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO:101; and/or encodes a region having at least 80% identity to the amino acid sequence of SEQ ID NOs: 80 and/or 96-98, optionally wherein the sequence identity of (a), (b), (c) and/or (d) may be at least 85%, or at least 90%, or it may be at least 95%, optionally the sequence identity may be 100%.

In some embodiments, a mutation in an endogenous NST1 gene and a mutation in an endogenous NST3 gene may be any mutation that results in a mutated NST1 gene and a mutated NST3 gene that can confer a reduced endocarp lignification, reduced endocarp lignification and reduced pit hardness, reduced seediness and/or a reduced feel of seediness phenotypes on a Prunus plant comprising the mutated NST1 gene and mutated NST3 gene, optionally wherein the mutation in the endogenous NST1 gene and mutation in the endogenous NST3 gene may be a null mutation. In some embodiments, the mutation in an endogenous NST1 gene and mutation in an endogenous NST3 gene may be a non-natural mutation. In some embodiments, a mutation (e.g., one or more mutations) in an endogenous NST1 gene and in an endogenous NST3 gene may be a point mutation. In some embodiments, a mutation may be a base deletion. In some embodiments, the at least one mutation in an endogenous NST1 gene and/or the at least one mutation in an endogenous NST3 gene may be a dominant-negative allele. In some embodiments, the mutation in an endogenous NST1 gene and/or the mutation in an endogenous NST3 gene in a plant may be a base deletion that results in a plant having a reduced endocarp lignification, reduced endocarp lignification and reduced pit hardness, reduced seediness and/or a reduced feel of seediness phenotypes. In some embodiments, the mutation in an endogenous NST1 gene and/or the at least one mutation in an endogenous NST3 gene in a Prunus plant may be a deletion that results in a dominant allele. For example, the mutation may be a deletion of 1 nucleotide to about 100 nucleotides, optionally 2, 3, 4, 5 nucleotides to about 100 nucleotides or any range or value therein, optionally at least 5 nucleotides, or at least 10 nucleotides, or at least 15 nucleotides, or at least 20 nucleotides, or at least 25 nucleotides, or at least 30 nucleotides, or at least 35 nucleotides, or at least 40 nucleotides, or at least 45 nucleotides, or at least 100 nucleotides, or any range or value therein (e.g., out-of-frame deletion or an in-frame deletion). In some embodiments, the mutation in the NST1 gene and the mutation in the NST3 gene results in a truncated NST1 polypeptide and a truncated NST3 polypeptide.

In some embodiments, a plant genome or plant genomic DNA is provided that comprises at least one non-natural mutation in an endogenous NAC SECONDARY WALL THICKENING PROMOTING FACTOR1 (NST1) gene and an endogenous NAC SECONDARY WALL THICKENING PROMOTING FACTOR3 (NST3) gene that encodes a NST1 polypeptide and a NST3 polypeptide, respectively. In other embodiments, a plant genome or plant genomic DNA is provided that comprises a mutated NAC SECONDARY WALL THICKENING PROMOTING FACTOR1 (NST1) gene and a mutated NAC SECONDARY WALL THICKENING PROMOTING FACTOR3 (NST3) gene having a nucleic acid sequence as described herein and/or encoding a mutated NST1 polypeptide and mutated NST3 polypeptide having an amino acid sequence as described herein.

In some embodiments, a guide nucleic acid (e.g., gRNA, gDNA, crRNA, crDNA) is provided that binds to a target site within an endogenous NAC SECONDARY WALL THICKENING PROMOTING FACTOR1 (NST1) gene, wherein the target site is in a region of the NST1 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences of SEQ ID NOs: 69, 70, 81, or 82; comprising a region having at least 80% sequence identity to a nucleotide sequence of any one of SEQ ID NOs: 72-76 and/or 84-87; encoding a polypeptide comprising a sequence having at least 80% sequence identity to the amino acid sequence of SEQ ID NO:71 and/or SEQ ID NO:83; and/or encoding a region having at least 80% identity to the amino acid sequence of SEQ ID NOs: 77-80, optionally wherein the sequence identity may be at least 85%, or at least 90%, or it may be at least 95%, optionally the sequence identity may be 100%. In some embodiments, a guide nucleic acid is provided that binds to a target site within an endogenous NST1 gene, wherein the guide nucleic acid comprises a spacer sequence have at least 90% sequence identity (e.g., 90, 91, 92, 93, 94, 95, 96, 97, 98, 99 or 100% sequence identity) to the sequence of SEQ ID NO: 110 and/or SEQ ID NO:111.

In some embodiments, a guide nucleic acid (e.g., gRNA, gDNA, crRNA, crDNA) is provided that binds to a target site within an endogenous NAC SECONDARY WALL THICKENING PROMOTING FACTOR3 (NST3) gene, wherein the target site is in a region of the NST3 gene having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences of SEQ ID NOs: 88, 89, 99, or 100; comprising a region having at least 80% sequence identity to a nucleotide sequence of any one of SEQ ID NOs: 91-95 and/or 102-105; encoding a polypeptide comprising a sequence having at least 80% sequence identity to the amino acid sequence of SEQ ID NO:90 and/or SEQ ID NO: 101; and/or encoding a region having at least 80% identity to the amino acid sequence of any one of SEQ ID NOs: 80 and/or 96-98, optionally wherein the sequence identity may be at least 85%, or at least 90%, or it may be at least 95%, optionally the sequence identity may be 100%. In some embodiments, a guide nucleic acid is provided that binds to a target site within an endogenous NST3 gene, wherein the guide nucleic acid comprises a spacer sequence have at least 90% sequence identity (e.g., 90, 91, 92, 93, 94, 95, 96, 97, 98, 99 or 100% sequence identity) to the sequence of SEQ ID NOs: 112 and/or SEQ ID NO:113.

In some embodiments, the target site within an endogenous NST1 gene to which a guide nucleic acid of the invention may bind may comprise a nucleotide sequence, or portion thereof, having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 72-76 and/or 84-87.

In some embodiments, the target site within an endogenous NST3 gene to which a guide nucleic acid of the invention may bind may comprise a nucleotide sequence, or portion thereof, having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 91-95 and/or 102-105.

Example spacer sequences useful with a guide that binds to a target site within an endogenous NST1 gene may comprise complementarity (or reverse complementarity) to a fragment or portion of a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 69, 70, 81, or 82, optionally at least about 80, 81, 82, 83, 84, 85, 86, 87, 88, 89, 90, 91, 92, 93, 94, 95, 96, 97, 98, 99 or 100% sequence identity (optionally at least 90% sequence identity or 95% sequence identity, or 100% sequence identity) to any one of the nucleotide sequences of SEQ ID NOs: 72-76 and/or 84-87; or a fragment or portion of a nucleotide sequence encoding a polypeptide comprising a sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to the amino acid sequence of SEQ ID NO:71 and/or SEQ ID NO: 83, and/or the amino acid sequence of any one of SEQ ID NOs: 77-80. In some embodiments, an example spacer sequence may comprise the nucleotide sequence of SEQ ID NO: 110 and/or SEQ ID NO:111, or a reverse complement thereof, or any combination thereof.

Example spacer sequences useful with a guide that binds to a target site within an endogenous NST3 gene may comprise complementarity to a fragment or portion of a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to any one of the nucleotide sequences of SEQ ID NOs: 88, 89, 99, or 100, optionally at least about 80, 81, 82, 83, 84, 85, 86, 87, 88, 89, 90, 91, 92, 93, 94, 95, 96, 97, 98, 99 or 100% sequence identity (optionally at least 90% sequence identity or 95% sequence identity, or 100% sequence identity) to any one of the nucleotide sequences of SEQ ID NOs: 91-95 and/or 102-105; or a fragment or portion of a nucleotide sequence encoding a polypeptide comprising a sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity, optionally the sequence identity may be at least 85%, or at least 90%, or it may be at least 95%, optionally the sequence identity may be 100%) to the amino acid sequence of SEQ ID NO:90 and/or SEQ ID NO:101; and/or the amino acid sequence of any one of SEQ ID NOs: 80 and/or 96-98. In some embodiments, an example spacer sequence may comprise the nucleotide sequence of SEQ ID NO:112 and/or SEQ ID NO:113, or a reverse complement thereof, or any combination thereof.

In some embodiments, a guide nucleic acid may comprise a spacer sequence having the nucleotide sequence of any one of SEQ ID NOs: 110, 111, 112, and/or 113, or a reverse complement thereof, or any combination thereof.

In some embodiments, a system is provided that comprises a guide nucleic acid of the present invention and a CRISPR-Cas effector protein that associates with the guide nucleic acid. In some embodiments, the system may further comprise a tracr nucleic acid that associates with the guide nucleic acid and a CRISPR-Cas effector protein, optionally wherein the tracr nucleic acid and the guide nucleic acid are covalently linked.

As used herein, “a CRISPR-Cas effector protein in association with a guide nucleic acid” refers to the complex that is formed between a CRISPR-Cas effector protein and a guide nucleic acid in order to direct the CRISPR-Cas effector protein to a target site within a gene.

In some embodiments, a gene editing system is provided, the gene editing system comprising a CRISPR-Cas effector protein in association with a guide nucleic acid, wherein the guide nucleic acid comprises at least two guide nucleic acids and at least one guide nucleic acid comprises a spacer sequence that binds to a NAC SECONDARY WALL THICKENING PROMOTING FACTOR1 (NST1) gene and at least one guide nucleic acid comprises a spacer sequence that binds to a NAC SECONDARY WALL THICKENING PROMOTING FACTOR3 (NST3) gene. In some embodiments, the guide nucleic acid comprises a spacer sequence that binds to a NST1 gene. In some embodiments, wherein the endogenous NST1 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO: 83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO:101; and/or encodes a region having at least 80% identity to the amino acid sequence of SEQ ID NOs: 80 and/or 96-98, optionally wherein the sequence identity of any one of (a), (b), (c) and/or (d) may be at least 85%, or at least 90%, or it may be at least 95%, optionally the sequence identity may be 100%. In some embodiments, the spacer sequence that binds to an NST1 gene binds to a region of the NST1 gene having at least 80% sequence identity to any one of the nucleotide sequences of SEQ ID NOs: 72-76 and/or 84-87, and the spacer sequence that binds to an NST3 gene binds to a region of the NST3 gene having at least 80% sequence identity to any one of the nucleotide sequences of SEQ ID NOs: 91-95 and/or 102-105.

In some embodiments, the guide nucleic acid of a gene editing system can comprise a spacer sequence that has complementarity (or reverse complementarity) to a region, portion or fragment of a NST1 nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences SEQ ID NOs: 69, 70, 81, or 82, or may encode a region, portion or fragment of a sequence having at least 80% sequence identity to the amino acid sequence of SEQ ID NO:71 and/or SEQ ID NO:83, and spacer sequence that has complementarity (or reverse complementarity) to a region, portion or fragment of a NST3 nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of the nucleotide sequences SEQ ID NOs: 88, 89, 99, or 100, or may encode a region, portion or fragment of a sequence having at least 80% sequence identity to the amino acid sequence of SEQ ID NO:90 and/or SEQ ID NO:101, optionally wherein the sequence identity may be at least 85%, or at least 90%, or it may be at least 95%, optionally the sequence identity may be 100%. In some embodiments, the spacer sequence that binds to a NAC SECONDARY WALL THICKENING PROMOTING FACTOR1 (NST1) gene comprises a nucleotide sequence of SEQ ID NO:110 and/or SEQ ID NO:111 and the spacer sequence that binds to a NAC SECONDARY WALL THICKENING PROMOTING FACTOR3 (NST3) gene comprises a nucleotide sequence of SEQ ID NO:112 and/or SEQ ID NO: 113. In some embodiments, a gene editing system may further comprise a tracr nucleic acid that associates with the guide nucleic acid and a CRISPR-Cas effector protein, optionally wherein the tracr nucleic acid and the guide nucleic acid are covalently linked.

The present invention further provides a complex comprising (a) a first guide nucleic acid and a second CRISPR-Cas effector protein comprising a cleavage domain, wherein the first guide nucleic acid binds to a target site within a NAC SECONDARY WALL THICKENING PROMOTING FACTOR1 (NST1) gene and (b) a second guide nucleic acid and a second CRISPR-Cas effector protein comprising a cleavage domain, wherein the second guide nucleic acid binds to a target site within a NAC SECONDARY WALL THICKENING PROMOTING FACTOR3 (NST3) gene, wherein the endogenous NST1 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO:83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (b) comprises a region having at least 80% sequence identity to a nucleotide sequence of any one of SEQ ID NOs: 91-95 and/or 102-105; (c) encodes a NST3 transcription factor polypeptide having at least 80% sequence identity to the amino acid sequence of SEQ ID NO:90 and/or SEQ ID NO:101; and/or encodes a region having at least 80% identity to the amino acid sequence of any one of SEQ ID NOs: 80 and/or 96-98, optionally wherein the sequence identity of (a), (b), (c) and/or (d) may be at least 90%, or it may be at least 95%, optionally the sequence identity may be 100%, wherein the cleavage domain of the first CRISPR-Cas effector protein cleaves a target strand in the NST1 gene and the cleavage domain of the second CRISPR-Cas effector protein cleaves a target strand in the NST3 gene.

Also provided herein are expression cassettes comprising a (a) a polynucleotide encoding CRISPR-Cas effector protein comprising a cleavage domain and (b) a guide nucleic acid that binds to a target site within a NAC SECONDARY WALL THICKENING PROMOTING FACTOR1 (NST1), wherein the guide nucleic acid comprises a spacer sequence that is complementary to and binds to the target site within the NST1 gene, the NST1 gene: (i) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 69, 70, 81, or 82; (ii) comprises a region having at least 80% sequence identity to any one of the nucleotide sequences of SEQ ID NOs: 72-76 and/or 84-87; (iii) encodes a NST1 transcription factor polypeptide having at least 80% sequence identity to the amino acid sequence of SEQ ID NO:71 and/or SEQ ID NO: 83; and/or (iv) encodes a region having at least 80% identity to the amino acid sequence of any one of SEQ ID NOs: 77-80, optionally wherein the sequence identity of (i), (ii), (iii) and/or (iv) may be at least 85%, or at least 90%, or it may be at least 95%, optionally the sequence identity may be 100%.

Further, provided herein are expression cassettes comprising a (a) a polynucleotide encoding CRISPR-Cas effector protein comprising a cleavage domain and (b) a guide nucleic acid that binds to a target site within a NAC SECONDARY WALL THICKENING PROMOTING FACTOR3 (NST3), wherein the guide nucleic acid comprises a spacer sequence that is complementary to and binds to the target site within the NST3 gene, the NST3 gene: (i) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (ii) comprises a region having at least 80% sequence identity to a nucleotide sequence of any one of SEQ ID NOs: 91-95 and/or 102-105; (iii) encodes a NST3 transcription factor polypeptide having at least 80% sequence identity to the amino acid sequence of SEQ ID NO:90 and/or SEQ ID NO: 101; and/or (iv) encodes a region having at least 80% identity to the amino acid sequence of SEQ ID NOs: 80 and/or 96-98, optionally wherein the sequence identity of (i), (ii), (iii) and/or (iv) may be at least 85%, or at least 90%, or it may be at least 95%, optionally the sequence identity may be 100%.

In some embodiments, the target site is in a region of the NST1 gene having at least 80% sequence identity to any one of the nucleotide sequences of SEQ ID NOs: 72-76 and/or 84-87, and wherein the target site is in a region of the NST3 gene having at least 80% sequence identity to any one of the nucleotide sequences of SEQ ID NOs: 91-95 and/or 102-105.

In some embodiments, a nucleic acid is provided that encodes a mutated NAC SECONDARY WALL THICKENING PROMOTING FACTOR1 (NST1) polypeptide, optionally wherein the mutation results in a truncated NST1 polypeptide. In some embodiments, a nucleic acid is provided that encodes a mutated NAC SECONDARY WALL THICKENING PROMOTING FACTOR1 (NST3) polypeptide, optionally wherein the mutation results in a truncated NST3 polypeptide. In some embodiments, the mutated nucleic acid comprises a null mutation or dominant-negative mutation. In some embodiments, the mutated nucleic acid comprises a non-natural mutation.

Further provided are Prunus plants or parts thereof comprising a mutated NST1 nucleic acid and a mutated NST3 nucleic acid as described herein. In some embodiments, a Prunus plant is provided that comprises a mutated NST1 nucleic acid and a mutated NST3 nucleic acid as described herein and exhibits a phenotype of reduced endocarp lignification, reduced endocarp lignification and reduced pit hardness, reduced seediness and/or a reduced feel of seediness as compared to a control Prunus plant that is devoid of the at least one mutation. In some embodiments, a Prunus plant or part thereof is provided that comprises a mutation in an endogenous NST1 gene and/or an endogenous NST3 gene, wherein the at least one mutation is a null mutation or dominant-negative mutation, optionally wherein the at least one mutation is a non-natural mutation.

In some embodiments, a method of the present invention may further comprise regenerating a Prunus plant from a plant cell or plant part comprising a mutation (e.g., one or more mutations) in an endogenous NST1 gene and an endogenous NST3 gene. In some embodiments, a Prunus plant comprising a mutation in an endogenous NST1 gene exhibits a phenotype of reduced endocarp lignification, reduced endocarp lignification and reduced pit hardness, reduced seediness and/or a reduced feel of seediness, as compared to a control Prunus plant that is devoid of the at least one mutation. In some embodiments, the mutation is a null mutation. In some embodiments, the mutation may be a non-natural mutation. In some embodiments, the mutation is a base deletion. In some embodiments, the mutation results in a dominant-negative allele. In some embodiments, the regenerated Prunus plant comprises a mutated NST1 gene and a mutated NST1 gene having at least 90% sequence identity (e.g., at least about 90, 91, 92, 93, 94, 95, 96, 97, 98, 99 or 100% sequence identity, optionally the sequence identity may be at least 95%, optionally the sequence identity may be 100%) to a mutated NST1 nucleic acid and a mutated NST3 nucleic acid as described herein.

An editing system useful with this invention can be any site-specific (sequence-specific) genome editing system now known or later developed, which system can introduce mutations in target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing a NST1 gene and a NST3 gene may comprise contacting a target nucleic acid (e.g., a target region of the NST1 gene and the NST3 gene) with a base-editing fusion protein (e.g., a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific DNA binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a NST1 gene and a NST3 gene may comprise contacting a target nucleic acid (e.g., a target region of the NST1 gene and a target region of the NST3 gene) with a sequence-specific DNA binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific DNA binding fusion protein to the target nucleic acid and the sequence-specific DNA binding fusion protein is capable of recruiting the deaminase fusion protein to the target nucleic acid via the peptide tag-affinity polypeptide interaction, thereby editing a locus within the target nucleic acid. In some embodiments, the sequence-specific DNA binding fusion protein may be fused to the affinity polypeptide that binds the peptide tag and the deaminase may be fuse to the peptide tag, thereby recruiting the deaminase to the sequence-specific DNA binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific DNA binding fusion proteins, deaminase fusion proteins and guides may be provided as ribonucleoproteins (RNPs).

In some embodiments, methods such as prime editing may be used to generate a mutation in an endogenous NST1 gene and an endogenous NST3 gene. In prime editing, RNA-dependent DNA polymerase (reverse transcriptase, RT) and reverse transcriptase templates (RT template) are used in combination with sequence specific nucleic acid binding domains that confer the ability to recognize and bind the target in a sequence-specific manner, and which can also cause a nick of the PAM-containing strand within the target. The nucleic acid binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantage of the various methods of recruiting proteins for use in the editing to the target site, such methods including both non-covalent and covalent interactions between the proteins and nucleic acids used in the selected process of genome editing.

In some embodiments, the mutation or modification of an endogenous NST1 gene and an endogenous NST3 gene may be a base deletion and/or a point mutation that produces a mutated endogenous NST1 gene and a mutated endogenous NST3 gene, wherein the mutated NST1 gene encodes a null mutation and the mutated endogenous NST3 gene encodes null mutation, optionally wherein the mutated NST1 gene encodes a C-terminally truncated NST1 polypeptide and the mutated endogenous NST3 gene encodes a C-terminally truncated NST3 polypeptide, optionally wherein the mutated/modified NST1 gene and the mutated/modified NST3 gene confers a phenotype of reduced endocarp lignification, reduced endocarp lignification and reduced pit hardness, reduced seediness and/or a reduced feel of seediness on a Prunus plant or part thereof comprising the mutated/modified NST1 gene and mutated/modified NST3 gene. In some embodiments, a plant part may be a cell. In some embodiments, the Prunus plant or plant part thereof may be any Prunus plant or part thereof as described herein. In some embodiments, a Prunus plant useful with this invention may be P. avium, P. armeniaca, P. cerasus, P. domestica, P. dulcis, P. mume, P. glandulosa, P. persica, or P. salcinia, optionally wherein the Prunus plant may be sweet cherry, sour cherry, peach, apricot, or plum. In some embodiments, a Prunus plant comprising a mutated NST1 gene may have a mutation in the dimerization domain and/or transactivating domain of the NST1 transcription factor polypeptide and comprising a mutated NST3 gene having a mutation in the dimerization domain and/or transactivating domain of the NST3 transcription factor polypeptide, may further comprise a reduced endocarp lignification, reduced endocarp lignification and reduced pit hardness, reduced seediness and/or a reduced feel of seediness phenotypes as compared to a control Prunus plant that is devoid of the mutated NST1 gene and the mutated NST3 gene. In some embodiments, a Prunus plant comprising a mutated endogenous NST1 gene and mutated endogenous NST3 gene exhibits a phenotype of reduced endocarp lignification, reduced endocarp lignification and reduced pit hardness, reduced seediness and/or a reduced feel of seediness phenotypes as compared to a control Prunus plant that is devoid of the mutation in the NST1 and in the NST3 genes.

In some embodiments, a mutation that is introduced into an endogenous NST1 gene and an endogenous NST3 gene is a null mutation. In some embodiments, a mutation that is introduced into an endogenous NST1 gene and an endogenous NST3 gene is dominant-negative mutation. In some embodiments, a mutation that is introduced into an endogenous NST1 gene and an endogenous NST3 gene may be a non-natural mutation. In some embodiments, a mutation that is introduced into an endogenous NST1 gene and an endogenous NST3 gene may be a deletion of at least one nucleotide, at least two consecutive nucleotides (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 consecutive base pairs, or any value or range therein), wherein the mutation may be located between 100 and 300 bases (or any range or value therein) upstream of the stop codon of the endogenous NST1 gene and the endogenous NST3 gene, optionally wherein the mutation may be a deletion or elimination of the dimerization domain of the NST1 polypeptide or may alter the ability of the dimerization domain to dimerize and wherein the mutation may be a deletion or elimination of the dimerization domain of the NST3 polypeptide or may alter the ability of the dimerization domain of the NST3 polypeptide to dimerize, optionally wherein the dimerization domain of the NST1 polypeptide and the NST3 polypeptide comprises the amino acid sequence VPPGFRFHPTEEELLQYYL (SEQ ID NO:79) or VPPGFRFHPTEEELLHYYL (SEQ ID NO: 98), e.g., an amino acid sequence comprising the sequence EEELL (SEQ ID NO:80). In some embodiments, a mutation may be located in and/or adjacent to the first exon of the endogenous NST1 gene encoding a NST1 transcription factor polypeptide and/or the at least one mutation may be located in and/or adjacent to the first exon of the endogenous NST3 gene encoding a NST3 transcription factor polypeptide. In some embodiments, a mutation may be located in and/or adjacent to the third exon of the endogenous NST1 gene encoding a NST1 transcription factor polypeptide and/or the at least one mutation may be located in and/or adjacent to the third exon of the endogenous NST3 gene encoding a NST3 transcription factor polypeptide.

In some embodiments, a sequence-specific nucleic acid binding domain (sequence-specific DNA binding domains) of an editing system useful with this invention can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein.

In some embodiments, a sequence-specific nucleic acid binding domain may be a CRISPR-Cas effector protein, optionally wherein the CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas 12 effector protein.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain, e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NO: 56 and SEQ ID NO:57 or the nucleotide sequences of SEQ ID NOs: 58-68.

In some embodiments, the CRISPR-Cas effector protein may be a Cas9 polypeptide derived from Streptococcus pyogenes and recognizes the PAM sequence motif NGG, NAG, NGA (Mali et al. (2013) Science 339 (6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al (2010) Science 327(5962): 167-170, and Deveau et al. (2008) J. Bacteriol. 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al. (2008) J. Bacteriol. 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif NGRRT (R=A or G). In some embodiments, the CRISPR-Cas effector protein may be a Cas9 polypeptide derived from S. aureus, which recognizes the PAM sequence motif NGRRV (R=A or G). In some embodiments, the CRISPR-Cas effector protein may be a Cas9 polypeptide that is derived from Neisseria meningitidis and recognizes the PAM sequence motif NGATT or NGCTT (R=A or G, V=A, G or C) (See, e.g., Hou et al (2013) Proc. Natl. Acad. Sci. USA 110 (39): 15644-15649). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease see, e.g., amino acid sequences of SEQ ID NOs: 1-17, nucleic acid sequences of SEQ ID NOs: 18-20. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas 12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. (2019) Nat. Biotechnol. 37:1070-1079, each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat or a mouse. Thus, in some embodiments, an cytosine deaminase useful with the invention may be about 70% to about 100% identical to a wild-type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:27, SEQ ID NO: 28 or SEQ ID NO:29). In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO:24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO: 23, SEQ ID NO:24, SEQ ID NO:25, SEQ ID NO:26, SEQ ID NO:27, SEQ ID NO:28 or SEQ ID NO:29). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode an uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

An “uracil glycosylase inhibitor” useful with the invention may be any protein that is capable of inhibiting an uracil-DNA glycosylase base-excision repair enzyme. In some embodiments, a UGI domain comprises a wild-type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:41 or a polypeptide having about 70% to about 99.5% sequence identity to the amino acid sequence of SEQ ID NO:41 (e.g., at least 80%, at least 85%, at least 90%, at least 92%, at least 95%, at least 96%, at least 97%, at least 98%, at least 99%, or at least 99.5% identical to the amino acid sequence of SEQ ID NO:41). For example, in some embodiments, a UGI domain may comprise a fragment of the amino acid sequence of SEQ ID NO:41 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:41. In some embodiments, a UGI domain may be a variant of a known UGI (e.g., SEQ ID NO:41) having about 70% to about 99.5% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild-type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild-type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild-type E. coli TadA comprises the amino acid sequence of SEQ ID NO: 30. In some embodiments, a mutated/evolved E. coli TadA* comprises the amino acid sequence of SEQ ID NOs: 31-40 (e.g., SEQ ID NOs: 31, 32, 33, 34, 35, 36, 37, 38, 39 or 40). In some embodiments, a polynucleotide encoding a TadA/TadA* may be codon optimized for expression in a plant.

A cytosine deaminase catalyzes cytosine deamination and results in a thymidine (through an uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt function; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

In some embodiments, templated editing may be used to reduce endocarp lignification, reduce seediness or a reduce the feel of seediness in a Prunus plant or part thereof. Templated editing includes, but is not limited to, PRIME editing and REDRAW editing. In some embodiments, unique enzymes may be used, such as SHARC (see e.g., PCT/US2024/018165). In general, templated editing comprises a Cas polypeptide, a reverse transcriptase, a guide RNA, a primer binding site and a reverse transcriptase template. In some embodiments, the reverse transcriptase may be a DNA polymerase. These components and their exact make up can vary depending on the type of templated editing that is used. For example, a guide RNA may be an “extended” guide RNA that comprises an extended portion (in addition to a spacer) that comprises the primer binding site and an edit to be incorporated into the target nucleic acid (e.g., reverse transcriptase template). Methods for templated editing are known in the art, see for example, U.S. Pat. Nos. 11,926,834, 11,643,652, U.S. patent application Ser. No. 17/142,570, U.S. patent application Ser. No. 17/078,919 and PCT/US2024/018165.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csxl7, Csxl4, Csx10, Csx16, CsaX, Csx3, Csxl, Csxl5, Csfl, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. (2007) Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild-type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild-type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a portion of a target nucleic acid (e.g., target DNA) (e.g., protospacer). In some embodiments, the spacer sequences is complementary to a portion of consecutive nucleotides of a NST1 gene or a portion of consecutive nucleotides of a NST3 gene, wherein the endogenous NST1 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 69, 70, 81, or 82; (b) comprises a region having at least 80% sequence identity to any one of the nucleotide sequences of SEQ ID NOs: 72-76 and/or 84-87; (c) encodes a NST1 transcription factor polypeptide having at least 80% sequence identity to the amino acid sequence of SEQ ID NO:71 and/or SEQ ID NO: 83; and/or (d) encodes a region having at least 80% identity to the amino acid sequence of any one of SEQ ID NOs: 77-80, and wherein the endogenous NST3 gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 98, 99 or 100% sequence identity) to any one of SEQ ID NOs: 88, 89, 99, or 100; (ii) comprises a region having at least 80% sequence identity to a nucleotide sequence of any one of SEQ ID NOs: 91-95 and/or 102-105; (iii) encodes a NST3 transcription factor polypeptide having at least 80% sequence identity to the amino acid sequence of SEQ ID NO:90 and/or SEQ ID NO:101; and/or (iv) encodes a region having at least 80% identity to the amino acid sequence of any one of SEQ ID NOs: 80 and/or 96-98, optionally wherein the sequence identity of any one of (a), (b), (c) and/or (d) may be at least 85%, or at least 90%, or it may be at least 95%, optionally the sequence identity may be 100%. A spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%)) to a target nucleic acid. In some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length. In some embodiments, a spacer sequence may comprise any one of the sequences of SEQ ID NOs: 110-113, or any combination thereof or reverse complement thereof.

In some embodiments, the 5′ region of a spacer sequence of a guide nucleic acid may be identical to a target DNA, while the 3′ region of the spacer may be substantially complementary to the target DNA (such as a spacer of a Type V CRISPR-Cas system), or the 3′ region of a spacer sequence of a guide nucleic acid may be identical to a target DNA, while the 5′ region of the spacer may be substantially complementary to the target DNA (such as a spacer of a Type II CRISPR-Cas system), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
      5′-NNNNNNNNNNNNNNNNNNN-3′ RNA Spacer
            |||||||||||||||||||
3′-AAANNNNNNNNNNNNNNNNNNN-5′ Target strand
      ||||
5′-TTTNNNNNNNNNNNNNNNNNNN-3′ Non-target strand
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems ((2015) Nature Reviews Microbiology 13:722-736). Guide structures and PAMs are described in by R. Barrangou ((2015) Genome Biol. 16:247).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. (2013) Nat. Methods 10:1116-1121; Jiang et al. (2013) Nat. Biotechnol. 31:233-239). In some embodiments, a computational approach can include performing BLAST searches of natural spacers to identify the original target DNA sequences in bacteriophages or plasmids and aligning these sequences to determine conserved sequences adjacent to the target sequence (Briner and Barrangou. (2014) Appl. Environ. Microbiol. 80:994-1001; Mojica et al. (2009) Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific nucleic acid binding domains, CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, an octapeptide sold under the tradename FLAG®, a strep tag or strep tag II, a V5 tag, and/or a VSV-G epitope. In some embodiments, a peptide tag may also include phosphorylated tyrosines in specific sequence contexts recognized by SH2 domains, characteristic consensus sequences containing phosphoserines recognized by 14-3-3 proteins, proline rich peptide motifs recognized by SH3 domains, PDZ protein interaction domains or the PDZ signal sequences, and an AGO hook motif from plants. Peptide tags are disclosed in WO 2018/136783 and US Patent Application Publication No. 2017/0219596, which are incorporated by reference for their disclosures of peptide tags. Any epitope that may be linked to a polypeptide and for which there is a corresponding affinity polypeptide that may be linked to another polypeptide may be used with this invention as a peptide tag. A peptide tag may comprise or be present in one copy or in 2 or more copies of the peptide tag (e.g., multimerized peptide tag or multimerized epitope) (e.g., about 2, 3, 4, 5, 6, 7, 8, 9, 10, 11, 12, 13, 14, 15, 16, 17, 18, 9, 20, 21, 22, 23, 24, or 25 or more peptide tags). When multimerized, the peptide tags may be fused directly to one another or they may be linked to one another via one or more amino acids (e.g., 1, 2, 3, 4, 5, 6, 7, 8, 9, 10, 11, 12, 13, 14, 15, 16, 17, 18, 19, 20 or more amino acids, optionally about 3 to about 10, about 4 to about 10, about 5 to about 10, about 5 to about 15, or about 5 to about 20 amino acids, and the like, and any value or range therein. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin, see, e.g., Sha et al. (2017) Protein Sci. 26 (5): 910-924; Gilbreth (2013) Curr. Opin. Struc. Biol. 22 (4): 413-420; and U.S. Pat. No. 9,982,053, each of which are incorporated by reference in their entireties for the teachings relevant to affibodies, anticalins, monobodies and/or DARPins. Example peptide tag sequences and their affinity polypeptides include, but are not limited to, the amino acid sequences of SEQ ID NOs: 42-44.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases). Example RNA recruiting motifs and their affinity polypeptides include, but are not limited to, the sequences of SEQ ID NOs: 45-55.

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together, e.g., dihydrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

Further provided herein are cells comprising one or more polynucleotides, guide nucleic acids, nucleic acid constructs, expression cassettes or vectors of the invention.

The nucleic acid constructs of the invention (e.g., a construct comprising a sequence specific nucleic acid binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids and/or their expression.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, collenchyma cells, sclerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

Any Prunus plant comprising an endogenous NST1 gene and endogenous NST3 gene, wherein when modified as described herein the modified NST1 gene and modified NST3 are capable of conferring a reduced endocarp lignification, reduced endocarp lignification and reduced pit hardness, reduced seediness and/or a reduced feel of seediness phenotypes in the plant (e.g., Prunus plant), is useful with this invention. Non-limiting examples of Prunus plants that may be modified as described herein may P. avium, P. armeniaca, P. cerasus, P. domestica, P. dulcis, P. mume, P. glandulosa, P. persica, or P. salcinia, optionally wherein the Prunus plant may be sweet cherry, sour cherry, peach, apricot, or plum.

The invention will now be described with reference to the following examples. It should be appreciated that these examples are not intended to limit the scope of the claims to the invention but rather are intended to be exemplary of certain embodiments. Any variations in the exemplified methods that occur to the skilled artisan are intended to fall within the scope of the invention.

### EXAMPLES

### Example 1: Targeted Editing of NST1 and NST3 Genes in Prunus Plants Using CRISPR-Based Gene Editing

This example demonstrates the targeted mutation of the NST1 and NST3 genes in Prunus plant cells, resulting in a reduction in lignification of the endocarp.

A Prunus variety is selected for transformation. Multiple guide RNAs were designed to target the first exon within the Prunus NST1 and NST3 genes. gRNAs comprising spacers having the nucleic acid sequence of SEQ ID NO:110 and SEQ ID NO:111 are used to target the Prunus NST1 gene and the gRNAs comprising spacers having the nucleic acid sequence of SEQ ID NO:112, and SEQ ID NO:113 are used to target the Prunus NST3 gene. These gRNAs are cloned into a CRISPR expression vector containing a Cas12a enzyme. Prunus plant cells are transformed using Agrobacterium-mediated transformation. Transformed cells are cultured on selective media containing kanamycin to encourage the growth of successfully transformed cells. After sufficient time, the regenerated shoots are transferred to rooting media.

Following transformation, genomic DNA is extracted from regenerated plants. PCR amplification of the target regions within NST1 and NST3 is performed, and the resulting PCR products are sequenced to confirm the presence of indel mutations at the target sites, which indicates successful gene editing. The edited plants are further screened for null mutations, characterized by the introduction of stop codons due to out-of-frame mutations or disruption of the dimerization domain due to in-frame edits, e.g., deletion of dimerization domains leading to dominant-negative alleles.

Sequencing analysis is used to confirm successful editing of the NST1 and NST3 loci.

A subset of regenerated plants with edits in NST1 and NST3 are grown to maturity, manually self-pollinated, and fruit collected to screen for heritability of the phenotype.

### Example 2: Role of NST1 and NST3 in Endocarp Lignification

NAC transcription factors, including NST1 and NST3, bind as a homodimer or heterodimer, and they act as conserved, key regulators of lignin deposition in plant development. Blackberry NST1 and NST3 (see, example WT blackberry NST1/NST3 coding sequences (e.g., SEQ ID NO:106 and SEQ ID NO:108) and polypeptide sequences (e.g., SEQ ID NO:107 and 109)) have been shown to be associated with a soft pit phenotype. In particular, edited blackberry NST1 and edited blackberry NST3 genomic sequences that lead to premature stop codons truncating the encoded NST1 and NST3 polypeptide sequences result in the reduction of lignification in the endocarp as measured by staining with phloroglucinol and as measured by the force required to compress blackberry pyrenes. See, edited blackberry plants in U.S. Pat. No. 12,297,441. This reduction in compressive force indicates a decrease in the lignification of the endocarp, which correlates with the intended phenotypic outcome of reduced seed hardness.

Edited NST1 and NST3 sequences (e.g., resulting in truncated NST1 and NST3 polypeptides) in Prunus species are hypothesized to prevent activation of downstream targets in situ, thereby preventing lignification of tissues of interest. In particular, as in blackberry (Rubus), edited NST1 and NST3 genes in Prunus species may result in plants exhibiting reduced endocarp lignification, reduced endocarp lignification, and reduced pit hardness as compared to a control plant that is devoid of the edited NST1 and NST3 sequences.

### Example 3: Prunus NST1 and Prunus NST3 Activate Secondary Cell Wall Biosynthesis Transcriptional Programs in Prunus Protoplasts

This example demonstrates that Prunus avium (sweet cherry) and Prunus persica (peach) NST1 and NST3, when overexpressed in cherry protoplasts, activate transcriptional programs associated with secondary cell wall biosynthesis, consistent with the role of NST1 and NST3 in regulating endocarp lignification in these species.

Protoplast Preparation and Transient Transformation. Prunus avium protoplasts were prepared from leaf tissue by enzymatic cell wall digestion. Briefly, young tissue from in vitro grown cherry plants was harvested and sliced with a scalpel before incubation in an enzymatic digestion buffer. The resulting protoplasts were washed twice with a transfection buffer and counted via a hemocytometer with yields between 30 and 60 million cells per gram of tissue. Protoplast populations had viability>90%. Protoplasts were transfected with 40% PEG in a deep 96-well plate with 250,000 protoplasts and 10 μg of plasmid per sample.

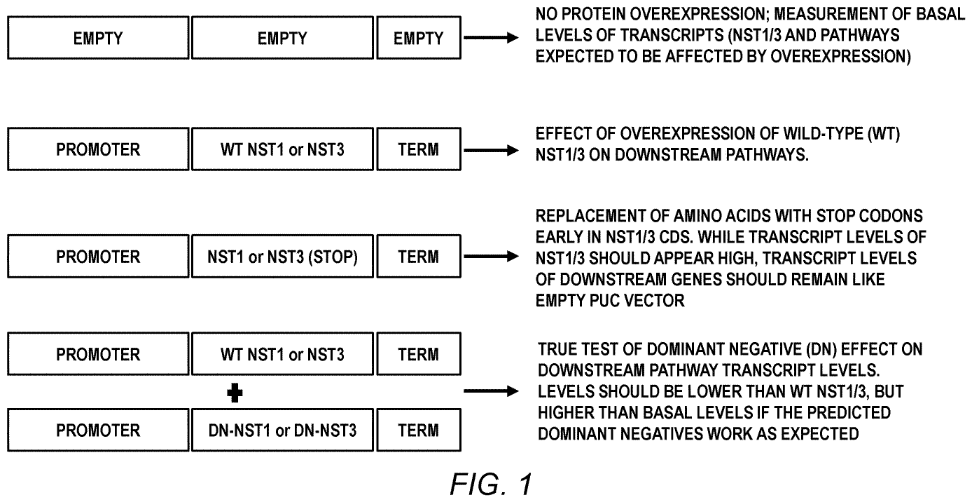

Protoplasts were transformed with expression constructs expressing wild-type cherry NST1 (SEQ ID NO:71 encoded by SEQ ID NO:70) and wild-type cherry NST3 (SEQ ID NO: 90 encoded by SEQ ID NO:89) polypeptides under control of a constitutive promoter; wild-type blackberry (Rubus sp.) NST1 (SEQ ID NO:107 encoded by SEQ ID NO:106); wild-type blackberry NST3 (SEQ ID NO:109 encoded by SEQ ID NO:108); wild-type peach NST1 (SEQ ID NO:83 encoded by SEQ ID NO:82); and wild-type peach NST3 (SEQ ID NO:101 encoded by SEQ ID NO:100) (FIG. 1). In addition, protoplasts were transformed with expression constructs expressing NST polypeptides truncated at the dimerization domain, in particular a truncated cherry NST1 polypeptide (SEQ ID NO:114 encoded by SEQ ID NO:125), truncated cherry NST3 polypeptide (SEQ ID NO:116 encoded by SEQ ID NO:127), truncated peach NST1 polypeptide (SEQ ID NO:118 encoded by SEQ ID NO:129), truncated peach NST3 polypeptide (SEQ ID NO:116 encoded by SEQ ID NO:131), truncated blackberry NST1 polypeptide (SEQ ID NO:121 encoded by SEQ ID NO:133), and truncated blackberry NST3 polypeptide (SEQ ID NO: 123 encoded by SEQ ID NO:135) (FIG. 1). Constructs for expressing a dominant-negative form of each protein comprising deletion of at least a portion of the dimerization domain were also prepared. These included a construct encoding a dominant-negative cherry NST1 polypeptide (SEQ ID NO:115 encoded by SEQ ID NO:126), dominant-negative cherry NST3 polypeptide (SEQ ID NO:117 encoded by SEQ ID NO:128), dominant-negative peach NST1 polypeptide (SEQ ID NO:119 encoded by SEQ ID NO:130), dominant-negative peach NST3 polypeptide (SEQ ID NO:120 encoded by SEQ ID NO: 132), dominant-negative blackberry NST1 polypeptide (SEQ ID NO:122 encoded by SEQ ID NO:134), and dominant-negative blackberry NST3 polypeptide (SEQ ID NO:124 encoded by SEQ ID NO:136) (FIG. 1). Control protoplasts received an empty vector (pUC19) under identical transformation conditions. At least four biological replicates (N=4) were prepared per treatment group. The transfected protoplasts were incubated for 72 hours at 25 °C before being spun down and resuspended in 50 μl DNA/RNA shield (Zymo).

RNA Extraction. Cells preserved in Zymo DNA/RNA Shield (approximately 100,000-250,000 cells) were submitted to Plasmidsaurus for processing. Total RNA was extracted using a bead-based extraction method. Post-extraction RNA concentration was measured by a fluorescence-based plate reader assay, and sample concentrations were normalized prior to library preparation.

Library Preparation and Sequencing. Messenger RNA (mRNA) was converted to complementary DNA (cDNA) by reverse transcription followed by second-strand synthesis. The resulting cDNA was processed by tagmentation, library indexing, and amplification to generate indexed sequencing libraries employing a 3′ end counting approach for quantification of differential gene expression. Indexed libraries were sequenced on an Illumina platform.

RNA-seq quantification. RNA-seq libraries were processed through the nf-core/rnaseq pipeline (Ewels et al. Nat. Biotechnol. 2020 March; 38 (3): 276-278) with the sweet cherry PavV1 reference genome and transcriptome annotation, and gene-level counts were taken from the salmon software mapping and quantification output (Patro et al. Nat. Methods 2017 April; 14 (4): 417-419).

Differential expression. Low-expression genes were filtered prior to model fitting (retaining genes with >=10 counts in at least 3 samples). Differential expression was performed with DESeq2 (Love et al. Genome Biol. 2014; 15:550) for each per-species experimental group: contrasts for cherry, blackberry, and peach NST1/NST3 wild-type and mutant constructs versus pUC19 transfection.

GO term enrichment. Per-contrast GO biological-process enrichment was run with topGO (Alexa & Rahnenfuhrer, R package version 2.56.0, 2024) using the weight01 algorithm, against a gene-to-GO annotation table for the reference. Overexpressed NST1 and NST3 transgenes were removed from the input DE gene lists prior to enrichment. Each contrast was tested on up-regulated genes at log₂ fold change (LFC)>1 (p_{adj}<0.05). Enrichment results were summarized across contrasts with aggregated and per-species bar plots for plant-type secondary cell wall biogenesis (GO:0009834) comparing-log 10 weightFisher p-values across treatment contrasts. This GO term is found as enrichment in multiple endocarp transcriptome studies (Yu et al. Int. J. Mol. Sci. 2023 Mar. 31; 24 (7): 6543; Wen et al. Horticulture 2025, 11:609; Xue et al. J. Integr. Plant Biol. 2026, 68:239-256). For GO:0009834, per-gene log 2 fold changes were aggregated across contrasts within each species and plotted as Log 2 Fold Change (LFC) boxplots, and pairwise species comparisons (Blackberry vs Cherry NST1/NST3 Dominant-negative vs pUC19) were rendered as scatter plots with the Pearson correlation coefficient.

Software. Analyses were run in R with DESeq2, topGO, and the tidyverse (dplyr, tidyr, ggplot2) packages.

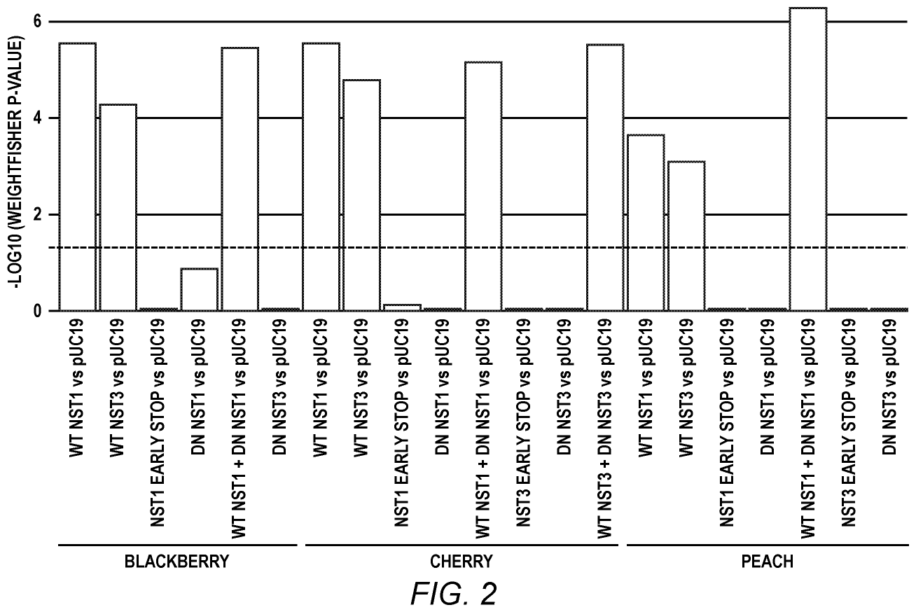

Gene Set Enrichment Results. Gene Ontology (GO) analysis showed that overexpression of blackberry, cherry, or peach wild-type NST1 or wild-type NST3 in cherry protoplasts produced strong enrichment of the plant-type secondary cell wall biogenesis genes (GO:0009834) (FIG. 2).

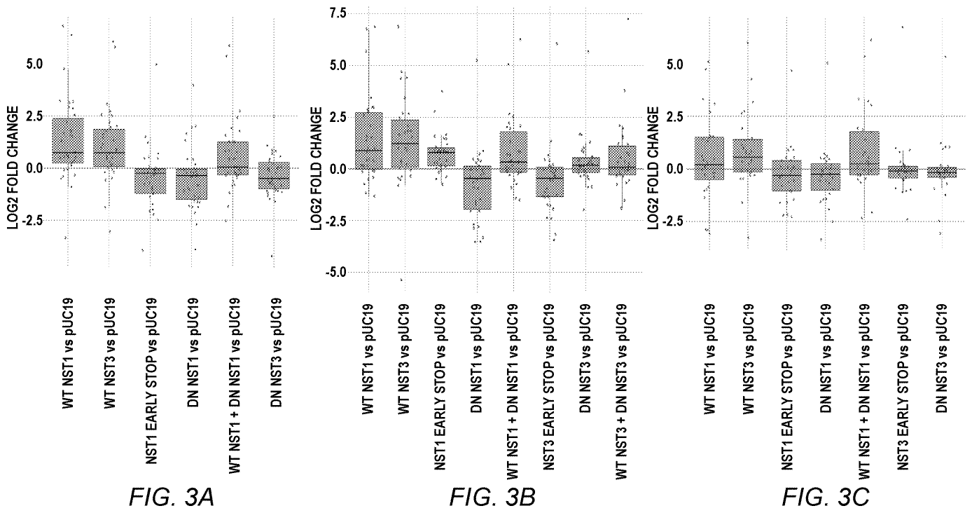

As shown in FIG. 3A, overexpression of wild-type blackberry NST1 (SEQ ID NO: 107) or wild-type blackberry NST3 (SEQ ID NO:109) in cherry protoplasts showed increased activation of genes in GO:0009834, whereas expression of dominant-negative blackberry NST1 (SEQ ID NO:122) or blackberry NST3 (SEQ ID NO:124) resulted in repression of gene expression in that pathway. Co-expression of wild-type blackberry NST1 (SEQ ID NO:107) and dominant-negative blackberry NST1 (SEQ ID NO:122) together resulted in canceling out of activation or repression of genes in GO:0009834. Further, blackberry NST1 containing an early stop codon (resulting in a truncated protein; SEQ ID NO: 121) showed minimal effect on gene expression for the GO:0009834 pathway.

Similar to blackberry, overexpression of wild-type cherry NST1 (SEQ ID NO:71) or wild-type cherry NST3 (SEQ ID NO:90) in cherry protoplasts showed increased activation of genes in GO:0009834 (FIG. 3B). Expression of the dominant-negative cherry NST1 (SEQ ID NO: 115) or dominant-negative cherry NST3 (SEQ ID NO:117) resulted in repression of gene expression in the GO:0009834 pathway. Co-expression of wild-type cherry NST1 (SEQ ID NO: 71) and dominant-negative cherry NST1 (SEQ ID NO:115) or co-expression of wild-type cherry NST3 (SEQ ID NO:90) and dominant-negative cherry NST3 (SEQ ID NO:117) resulted in cancelling out of activation or repression of the GO:0009834 pathway genes. Further, expression of cherry NST1 containing an early stop codon (resulting in a truncated protein; SEQ ID NO:114) or truncated cherry NST3 (SEQ ID NO:116) showed minimal effect on gene expression for the GO:0009834 pathway.

Expression of peach NST1 and NST3 proteins in cherry protoplasts (FIG. 3C) was also tested. These results showed that overexpression of wild-type peach NST1 (SEQ ID NO: 83) or wild-type peach NST3 (SEQ ID NO:101) in cherry protoplasts showed activation of genes in GO:0009834. Expression of dominant-negative peach NST1 (SEQ ID NO:119) or dominant-negative peach NST3 (SEQ ID NO:120) resulted in repression of gene expression in the GO:0009834 pathway. Co-expression of wild-type peach NST1 (SEQ ID NO: 83) and dominant-negative peach NST1 (SEQ ID NO:119) together did not result in canceling out of gene activation or repression. However, expression of truncated peach NST1 (SEQ ID NO:118) attenuated but did not reverse wild-type-driven activation of GO:0009834 genes, and expression of truncated peach NST3 (SEQ ID NO:116) showed minimal effect on gene expression in the pathway.

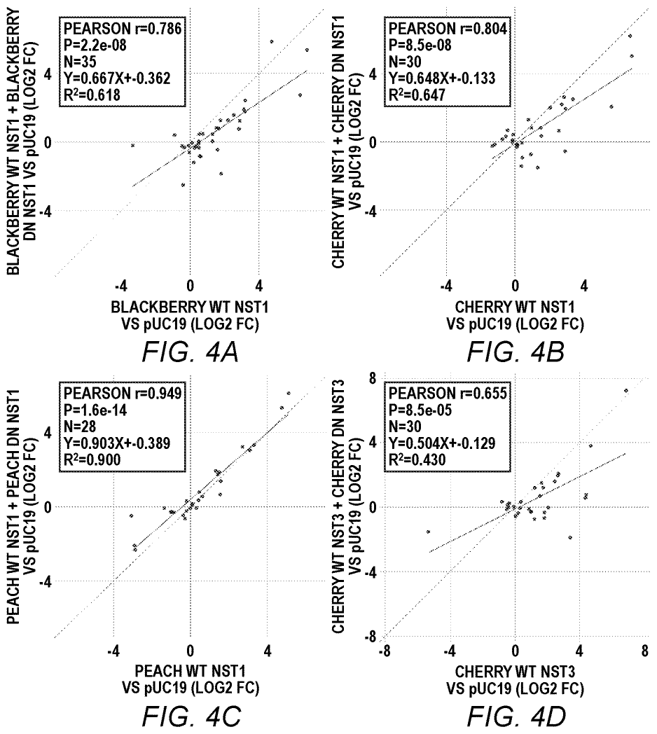

Pairwise species comparisons of log 2 Fold change of secondary cell wall biogenesis genes (GO:0009834) for wild-type NST1 vs wild-type NST1+dominant-negative NST1 (both relative to pUC19) across blackberry (FIG. 4A), cherry (FIG. 4B), and peach (FIG. 4C), as well as wild-type cherry NST3 vs wild-type cherry NST3+dominant-negative cherry NST3 (FIG. 4D) showed strong positive Pearson correlations (r=0.786 Blackberry NST1, r=0.804 Cherry NST1, r=0.949 Peach NST1, r=0.655 Cherry NST3) with regression slopes well below 1 for blackberry and cherry indicating that co-expression of the dominant-negative polypeptides attenuates but does not reverse wild-type-driven activation of GO:0009834 genes, consistent with the cancelling-out effect observed in the per-species analyses.

These results demonstrate that Prunus NST1 and Prunus NST3 function as activators of the secondary cell wall biosynthesis transcriptional program in Prunus avium cells, consistent with the established role of NST transcription factors in regulating secondary cell wall formation and endocarp lignification. The conservation of NST1/NST3-regulated secondary cell wall transcriptional programs across Rubus and Prunus genera is consistent with a conserved role for NST transcription factors in regulating endocarp lignification across Rosaceae. These data support the expectation that modification of endogenous NST1 and NST3 genes in Prunus species, as described herein, will produce phenotypes of reduced endocarp lignification and/or reduced pit hardness analogous to the reduced endocarp lignification and reduced seediness phenotypes observed in edited Rubus plants.

The testing described above may also be performed with NST1 and NST3 of other Prunus species, such as Prunus mume (Japanese apricot, see, e.g., GENBANK Accession No. XP_008239266.1 for NST3 and GENBANK Accession No. XP_008233055.1 for NST1), and Prunus dulcis (almond, see, e.g., GENBANK Accession No. XP_034215953.1 for NST3 and GENBANK Accession No. XP 034203801.1 for NST1).

In some jurisdictions, products obtained exclusively by essentially biological processes are excluded from patent protection. Accordingly, the claimed plants, plant parts and cells and their progeny can be defined as directed only to those plants, plant parts and cells and their progeny which are obtained by technical intervention (regardless of any further propagation through crossing and selection). An embodiment of the invention is directed at plants, or plant parts or progeny produced or obtainable using gene editing technology by introducing through stable or transient transformation an RNA-specific CRISPR/Cas system directed against or targeting a NST1 nucleotide sequence and targeting a NST3 nucleotide sequence, or one or more polynucleotide sequence(s) encoding said RNA-specific CRISPR/Cas system into the plant or the plant part. Alternatively, the subject matter excluded from patentability may be disclaimed. An embodiment of the invention is directed at plants, part of plants or progeny thereof comprising the alterations of the NST1 gene and the NST3 gene as elsewhere herein described, provided that the plants, parts or plants or progeny are not obtained exclusively through essentially biological processes, wherein essentially biological processes are processes for the production of plants or animals if they consist entirely of natural phenomena such as crossing or selection.

The foregoing is illustrative of the present invention and is not to be construed as limiting thereof. The invention is defined by the following claims, with equivalents of the claims to be included therein.

What is claimed is:

1. A Prunus plant or plant part thereof comprising at least one mutation (e.g., one or more) in at least one endogenous NAC SECONDARY WALL THICKENING PROMOTING FACTOR1 (NST1) gene encoding a NST1 transcription factor polypeptide and at least one mutation in at least one endogenous NAC SECONDARY WALL THICKENING PROMOTING FACTOR3 (NST3) gene encoding a NST3 transcription factor polypeptide, wherein the at least one mutation in the at least one endogenous NST1 gene and/or the at least one mutation in the at least one endogenous NST3 gene is a null mutation or a dominant-negative mutation.

2. The Prunus plant or part thereof of claim 1, wherein the null mutation results in the production of a truncated polypeptide.

3. The Prunus plant or part thereof of claim 1, wherein the null mutation results in the production of a non-functional or no NST1 transcription factor polypeptide and/or a non-functional or no NST3 transcription factor polypeptide.

4. The Prunus plant or part thereof of claim 1, wherein the at least one mutation is located in and/or adjacent to the first exon of the endogenous NST1 gene encoding a NST1 transcription factor polypeptide and/or the at least one mutation is located in and/or adjacent to the first exon of the endogenous NST3 gene encoding a NST3 transcription factor polypeptide.

5. The Prunus plant or part thereof of claim 1, wherein the at least one mutation is located in the first exon of the endogenous NST1 gene and/or is located in the first exon of the endogenous NST3 gene.

6. The Prunus plant or part thereof of claim 1, wherein the endogenous NST1 gene comprises a nucleic acid sequence having at least 80% sequence identity to any one of SEQ ID NOs: 69, 70, 81, or 82, or encodes an amino acid sequence having at least 80% sequence identity (e.g., at least 90% sequence identity) to SEQ ID NO:71 and/or SEQ ID NO:83, and the endogenous NST3 gene comprises a nucleic acid sequence having at least 80% sequence identity to SEQ ID NOs: 88, 89, 99, or 100, or encodes an amino acid sequence having at least 80% sequence identity (e.g., at least 90% sequence identity) to SEQ ID NO:90 and/or SEQ ID NO: 101.

7. The Prunus plant or part thereof of claim 1, wherein the at least one mutation in at least one endogenous NST1 gene and/or the at least one mutation in at least one endogenous NST3 gene comprises a base substitution, a base deletion, and/or a base insertion.

8. The Prunus plant or part thereof of claim 1, wherein the at least one mutation in at least one endogenous NST1 gene is an out-of-frame insertion or an out-of-frame deletion and/or the at least one mutation in an endogenous NST3 gene is an out-of-frame insertion or an out-of-frame deletion.

9. The Prunus plant or part thereof of claim 1, wherein the at least one mutation in at least one endogenous NST1 gene is an in-frame insertion or an in-frame deletion and/or the at least one mutation in an endogenous NST3 gene is an in-frame insertion or an in-frame deletion.

10. The Prunus plant or part thereof of claim 1, wherein the at least one mutation in at least one endogenous NST1 gene results in a deletion or insertion of one or more base pairs located in a region having at least 80% sequence identity to any one of the nucleotide sequences of SEQ ID NOs: 72-76 and/or 84-87 and the at least one mutation in at least one endogenous NST3 gene results in a deletion or insertion of one or more base pairs located in a region having at least 80% sequence identity to any one of the nucleotide sequences of SEQ ID NOs: 91-95 and/or 102-105.

11. The Prunus plant or plant part thereof of claim 1, wherein the at least one mutation in at least one NST1 endogenous gene and the at least one mutation in at least one endogenous NST3 gene results in the plant or part thereof that exhibits (1) reduced endocarp lignification or (2) reduced endocarp lignification and reduced pit hardness.

12. The Prunus plant or part thereof of claim 1, wherein the Prunus plant is P. avium, P. armeniaca, P. cerasus, P. domestica, P. dulcis, P. mume, P. glandulosa, P. persica, or P. salcinia.

13. The Prunus plant or part thereof of claim 1, wherein the Prunus plant is sweet cherry, sour cherry, peach, apricot, or plum.

14. The Prunus plant or part thereof of claim 1, wherein the Prunus plant is P. avium, P. persica, or P. armeniaca.

15. A Prunus plant regenerated from the plant part of claim 1.

16. The Prunus plant of claim 15, wherein the plant exhibits (1) reduced endocarp lignification or (2) reduced endocarp lignification and reduced pit hardness.

17. The Prunus plant of claim 15, wherein the Prunus plant is P. avium, P. armeniaca, P. cerasus, P. domestica, P. dulcis, P. mume, P. glandulosa, P. persica, or P. salcinia, optionally wherein the Prunus plant is sweet cherry, sour cherry, peach, apricot, or plum.

18. A method for editing a specific site in the genome of a Prunus plant cell, the method comprising: cleaving, in a site-specific manner, a target site within an endogenous NAC SECONDARY WALL THICKENING PROMOTING FACTOR1 (NST1) gene in the Prunus plant cell and a target site within an endogenous NAC SECONDARY WALL THICKENING PROMOTING FACTOR3 (NST3) gene in the Prunus plant cell, wherein the target site is in a region of the NST1 gene having at least 80% sequence identity to any one of the nucleotide sequences of SEQ ID NOs: 72-76 and/or 84-87, and the target site is in a region of the NST3 gene having at least 80% sequence identity to any one of the nucleotide sequences of SEQ ID NOs: 91-95 and/or 102-105, thereby generating an edit in the endogenous NST1 gene and in the endogenous NST3 gene of the Prunus plant cell and the edit results in a null mutation or a dominant-negative mutation.

19. The method of claim 18, wherein the endogenous NST1 gene comprises a nucleic acid sequence having at least 80% sequence identity to any one of SEQ ID NOs: 69, 70, 81, or 82 or encodes an amino acid sequence having at least 80% sequence identity (e.g., at least 90% sequence identity) to SEQ ID NO:71 and/or SEQ ID NO:83, and the endogenous NST3 gene comprises a nucleic acid sequence having at least 80% sequence identity to any one of SEQ ID NOs: 88, 89, 99, or 100 or encodes an amino acid sequence having at least 80% sequence identity (e.g., at least 90% sequence identity) to SEQ ID NO:90 and/or SEQ ID NO:101.

20. The method of claim 18, wherein the edit in the endogenous NST1 gene is in Exon 1 of the endogenous NST1 gene and the edit in the endogenous NST3 gene is in Exon 1 of the endogenous NST3 gene.

21. The method of claim 18, further comprising regenerating a Prunus plant from the Prunus plant cell comprising the edit in the endogenous NST1 gene and in the endogenous NST3 gene to produce a Prunus plant comprising the edit in its endogenous NST1 gene and endogenous NST3 gene, wherein the Prunus plant comprising the edit in its endogenous NST1 gene and endogenous NST3 gene exhibits (1) reduced endocarp lignification or (2) reduced endocarp lignification and reduced pit hardness as compared to a control Prunus plant that is devoid of the edit.

22. A method for producing a Prunus plant or part thereof comprising at least one cell having a mutation in an endogenous NAC SECONDARY WALL THICKENING PROMOTING FACTOR1 (NST1) gene and a mutation in an endogenous NAC SECONDARY WALL THICKENING PROMOTING FACTOR1 (NST3) gene, the method comprising
contacting a target site within the endogenous NST1 gene and a target site within the endogenous NST3 gene in the Prunus plant or part thereof with a nuclease comprising a cleavage domain and a nucleic acid binding domain, wherein the nucleic acid binding domain of the nuclease binds to a target site within the endogenous NST1 gene and a target site within the endogenous NST3 gene, and/or
contacting a target site within the endogenous NST1 gene and a target site within the endogenous NST3 gene in the Prunus plant or part thereof with a first nuclease and a second nuclease each of which comprise a cleavage domain and a nucleic acid binding domain, wherein the nucleic acid binding domain of the first nuclease binds to a target site within the endogenous NST1 gene and the nucleic acid binding domain of the second nuclease binds to a target site within the endogenous NST3 gene,
wherein the target site is in a region of the NST1 gene having at least 80% sequence identity to any one of the nucleotide sequences of SEQ ID NOs: 72-76 and/or 84-87, and the target site is in a region of the NST3 gene having at least 80% sequence identity to any one of the nucleotide sequences of SEQ ID NOs: 91-95 and/or 102-105, thereby producing a Prunus plant or part thereof comprising at least one cell having a mutation in the endogenous NST1 gene and in the endogenous NST3 gene, wherein the mutation in the endogenous NST1 gene and/or in the endogenous NST3 gene is a null mutation or a dominant allele.

23. The method of claim 22, wherein the endogenous NST1 gene comprises a nucleic acid sequence having at least 80% sequence identity to any one of SEQ ID NOs: 69, 70, 81, or 82 or encodes an amino acid sequence having at least 80% sequence identity (e.g., at least 90% sequence identity) to SEQ ID NO:71 and/or SEQ ID NO:83, and the endogenous NST3 gene comprises a nucleic acid sequence having at least 80% sequence identity to any one of SEQ ID NOs: 88, 89, 99, or 100 or encodes an amino acid sequence having at least 80% sequence identity (e.g., at least 90% sequence identity) to SEQ ID NO:90 and/or SEQ ID NO:101.

24. The method of claim 22, wherein the mutation that is introduced into the endogenous NST1 gene is located in the 5′ region of the endogenous NST1 gene and the mutation that is introduced into the endogenous NST3 gene is located in the 5′ region of the endogenous NST3 gene.

25. A Prunus plant produced by the method of claim 22, wherein the Prunus plant exhibits (1) reduced endocarp lignification, or (2) reduced endocarp lignification and reduced pit hardness as compared to a control plant.

26. The Prunus plant of claim 25, wherein the Prunus plant comprises a null mutation in an NST1 gene and in an NST3 gene.

27. The Prunus plant of claim 25, wherein the Prunus plant comprises a dominant-negative mutation in an NST1 gene and in an NST3 gene.
